SEQUENCE LISTING (8 sequences)

SEQ ID NO: 1 - DNA, 20 bp, artificial sequence
  Synthetic oligonucleotide MDR1/ABCB1 5′-3′ primer
cccatcattgcaatagcagg

SEQ ID NO: 2 - DNA, 20 bp, artificial sequence
  Synthetic oligonucleotide MRP1/ABCC1 5′-3′ primer
atgtcacgtggaataccagc

SEQ ID NO: 3 - DNA, 19 bp, artificial sequence
  Synthetic oligonucleotide MRP2/ABCC2 5′-3′ primer
acagaggctggtggcaacc

SEQ ID NO: 4 - DNA, 22 bp, artificial sequence
  Synthetic oligonucleotide BCRP/ABCG2 5′-3′ primer
agatgggtttccaagcgttcat

SEQ ID NO: 5 - DNA, 21 bp, artificial sequence
  Synthetic oligonucleotide MDR1/ABCB1 3′-5′ primer
tgttcaaacttctgctcctga

SEQ ID NO: 6 - DNA, 20 bp, artificial sequence
  Synthetic oligonucleotide MRP1/ABCC1 3′-5′ primer
gaagactgaactcccttcct

SEQ ID NO: 7 - DNA, 25 bp, artificial sequence
  Synthetic oligonucleotide MRP2/ABCC2 3′-5′ primer
accattaccttgtcactgtccatga

SEQ ID NO: 8 - DNA, 23 bp, artificial sequence
  Synthetic oligonucleotide BCRP/ABCG2 3′-5′ primer
ccagtcccagtacgactgtgaca

(19) United States
(12) Patent Application Publication
(10) Pub. No.: US 2012/0309728 A1
(43) Pub. Date: Dec. 6, 2012

(54) NEW STEROID INHIBITORS OF PGP FOR USE FOR INHIBITING MULTIDRUG RESISTANCE

(76) Inventors: Catherine Grenot, Lyon Cedex 08 (FR); Claude Cuilleron, Lyon Cedex 08 (FR)

(21) Appl. No.: 13/516,860

(22) PCT Filed: Dec. 17, 2010
(86) PCT No.: PCT/EP2010/070141

§ 371 Date: Aug. 9, 2012

(30) Foreign Application Priority Data
Dec. 18, 2009 (EP) 09306282.6

(51) Int. Cl. A61K 31/575; C07J 17/00; C07J 9/00; A61K 31/58

(52) U.S. Cl. 514/172; 549/421; 552/553; 514/182; 514/179; 552/549

(57) ABSTRACT

The present invention relates to a compound of formula (I) for its use for reversing or inhibiting multidrug resistance.

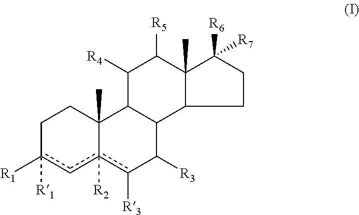

The present invention concerns new steroid inhibitors of Pgp for use for inhibiting multidrug resistance.

Reversion of the multidrug resistance phenotype (MDR phenotype) remains a major goal for many cancer research groups in fundamental and industrial fields. Different families of molecules have been tested for their ability to reverse MDR phenotype. In the first attempts, agents already in use for other medical indications (such as verapamil or cyclosporine) were tested as well as a large variety of chemical compounds having in common a predominantly hydrophobic character. Comparisons of these compounds and of their reversal activities have revealed structural features increasing the inhibitory effects (J. Robert and C. Jarry, Multidrug resistance reversal agents, J Med Chem 46 (2003) 4805-4817). Despite the great number of published active structures, significant improvements remain to be accomplished to decrease enough the side-effects to allow the development of efficient drugs.

The main target of these modulators is Pgp, a transmembrane protein overexpressed in many tumor cells treated by cytotoxic drugs. However, a major limit to the development of powerful inhibitors of drug efflux remains the lack of knowledge on the mechanism of expulsion. Drug binding, photoaffinity labelling and mutagenesis experiments have brought some informations on the localization of substrate and inhibitor binding sites on Pgp (M. Peer et al., Mini Rev in Med Chem 5 (2005) 165-172). Attempts to inhibit drug efflux were also made by designing anti-sense polynucleotides to down regulate the Pgp gene in tumor cells but their safe delivery to cancer cells in vivo remains difficult.

Modulatory effects have been described for diverse families of steroid structures, such as natural hormones (progesterone and medroxyprogesterone) (K. M. Barnes et al. Biochemistry 35 (1996) 4820-4827), steroid drugs (RU38486) (D. J. Gruol et al., Cancer Res 54 (1994) 3088-3091) or synthetic steroids (RU 49953) (F. J. Perez-Victoria et al., Cell Mol Life Sci 60 (2003) 526-535). Owing to their important physiological or pharmacological activities, these families of molecules have been intensively studied and their physiologic, pharmacologic and metabolic properties are well known as well as their toxicities, the structure of their biological targets, and their mechanisms of transport. Moreover, steroids are multifunctional molecules authorizing a well-established chemistry of multi-functionalization which can easily lead to pseudo-combinatorial structural modifications for the optimization of pharmacological activities.

Numerous derivatives acting on the reversion of MDR phenotype in cellular in vitro models have been described. The most efficient modulators could be classified into structurally different species (E. Teodori et al., II Farmaco 57 (2002) 385-415). Among them, calcium blocking agents such as verapamil, diltiazem or nifedipine and immunosupressants such as cyclosporine, were found to inhibit Pgp. However, when employed in clinical trials, they showed too low inhibitory activity and proved to be toxic at high doses.

Various problems have been encountered in the development of Pgp inhibitors including: i) lack of specificity versus other proteins; ii) intrinsic pharmacological activity; iii) weak in vivo accessibility for Pgp binding sites; iv) strong intrusion within the physiological role of Pgp which is present in normal tissues such as blood-brain barrier or liver and kidney (toxics or drugs elimination); v) toxic side effects (J. Robert, Expert Opin Investig Drugs 7 (1998) 929-939).

The aim of the present invention is to provide new Pgp inhibitors having an intrinsic pharmacological activity with no toxic side effects.

Another aim of the present invention is to provide new Pgp inhibitors having a strong in vivo affinity for Pgp binding sites.

Another aim of the present invention is to provide compounds able to reverse the multidrug resistance phenotype.

The present invention relates to a compound of formula (I): wherein

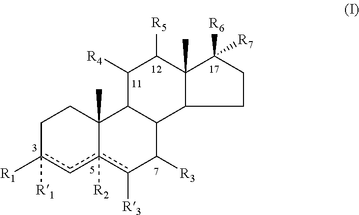

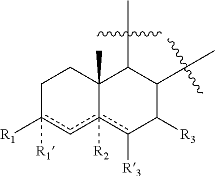

(A) is selected from (Ia), (Ib), (Ic) or (Id):

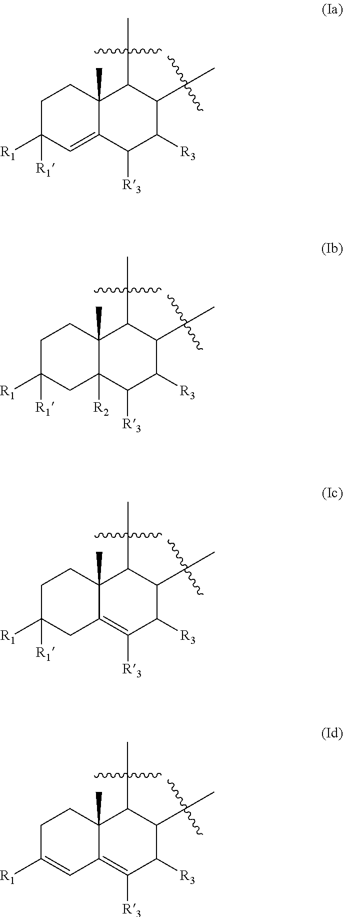

R₁ and R₁′ are each independently selected from H, OR₁₉, OC(═O)Ar, OSiR₁₅R₁₆R₁₇, and NR₁₈C(═O)Ar, or together with the carbon atom to which they are attached form ═O, or a 5 to 7 membered heterocyclyl;
provided that when (A) is (Id), R¹ cannot be ═O;
R₂ is H;
R₃ and R₃′ are each independently selected from H, OH, (CH₂)ₘOR₈, OR₈, ((OCH₂)₂)ₘOR₈, O(CH₂)ₘOR₈, OC(═O)Ar, NHC(═O)Ar, and NHC(═O)(CH₂)ₙAr, wherein said Ar groups are optionally substituted by one to three groups selected from OH, NO₂, N₃, NH₂, and N(CH₃)₂;
R₄ is H, OR₈, O(CH₂)ₘOR₈, ((OCH₂)₂)ₘOR₈, OC(═O)Ar or C(═O)CH₂NH(CH₂)ₜR₈;
R₅ is H, OR₉, OR₈, O(CH₂)ₘOR₈, ((OCH₂)₂)ₘOR₈, or OC(═O)Ar, wherein said Ar group is optionally substituted by one to three NO₂;
R₆ and R₇ are each independently selected from H, CR₁₀R₁₁R₁₂, O(═O)R₁₃, and OR₁₄;
R₈ is a 5 to 7 membered heterocyclyl, (CH₂)ₛCN or (CH₂)ₛNHC(═O)Ar, wherein said Ar is optionally substituted by one to three groups selected from NO₂, N₃ or OH;
R₉ is a 5 to 7 membered heterocyclyl or (CH₂)ₚOAlk;
R₁₀, R₁₁, and R₁₂ are each independently selected from H, OH, C₁-C₆ alkyl, OC(═O)Ar, OSiR₁₅R₁₆R₁₇, (CH₂)ₚOAlk and (CH₂)ₚC(═O)OAlk, or two of R₁₀, R₁₁, R₁₂ together form with the carbon atom to which they are attached a 5 to 7 membered heterocyclyl;
R₁₃ is C₁-C₆ alkyl, (CH₂)ᵣOHet, (CH₂)ᵣSAr or (CH₂)ᵣSAlk,
R₁₄ is H, ((OCH₂)₂)ₘOR₈, O(CH₂)ₘOR₈, a 5 to 7 membered heterocyclyl or C(═O)Ar;
R₁₅, R₁₆ and R₁₇ are each independently selected from C₁-C₆ alkyl;
R₁₈ is H or C₁-C₆ alkyl;
R₁₉ is H, a 5 to 7 membered heterocyclyl or (CH₂)ₛNHC(═O)Ar, wherein said Ar is optionally substituted by one to three groups selected from NO₂, N₃ or OH; and
m, n, p, q, r, s and t are each independently selected from 1, 2, 3 or 4; or its pharmaceutically acceptable salts, hydrates or hydrated salts or its polymorphic crystalline structures, racemates, diastereoisomers or enantiomers,
for its use for reversing or inhibiting multidrug resistance.

Preferably, in formula (I), when R₆ is other than H and R₇ is H, then at least one of R₃, R′₃, R₄ and/or R₅ is other than H, and when R₆ is COCH₃ and R₇ is OH, then at least one of R₃, R′₃, R₄ and/or R₅ is other than H.

Multidrug resistance (MDR) is a major limit for chemotherapy treatments of cancers. The predominant MDR mechanism is a reduced cytotoxic drug accumulation consecutive to the active efflux of drugs by energy-dependent transporters belonging to the ATP-binding cassette (ABC) family. The major contribution comes from P-glycoprotein (Pgp or ABCB1) encoded by MDR1 gene, which exports xenobiotics out of cells, as well as a wide variety of cytotoxic drugs (E. Teodori et al., Current Drug Targets 7 (2006) 896-909) and is often found overexpressed in resistant tumors. Two other proteins, MRP1 (Multidrug Resistance Protein 1, ABCC1) and BCRP (Breast Cancer Resistance Protein, ABCG1) may also often contribute, although to a lower extent, to the drug efflux.

Pgp is a glycoprotein of 1280 amino acids organized in two domains of 610 amino acids joined by a linker region of 60 residues. Each domain contains six transmembrane segments, separated by hydrophilic loops and a cytoplasmic hydrophilic nucleotide-binding domain (NBD) containing the well-conserved Walker A and B sequence motifs characterizing ATP binding sites (M. M. Gottesman et al., Nat Rev Cancer 21 (2002) 48-58). Photoaffinity labeling and mutagenesis experiments have suggested that the drug binding domain is located within the transmembrane domains in both halves of the protein (E. P. Bruggemann et al., J Biol Chem 264 (1989) 15483-15488; L. M. Greenberger, J Biol Chem, 268 (1993) 11417-11425; and B. Isenberg et al., Eur. J. Biochem. 268 (2001) 2629-2634). On the other hand, mutations and cross-linking experiments showed that the drug binding site encompassed the 4-6 and 10-12 trans-membrane helices and identified amino acids belonging to a common binding site in this region (T. W. Loo and D. M. Clarke, J Biol Chem 276 (2001) 36877-36880). Experiments based on mutagenesis, cysteine scanning and amino acids cross-linking led to a hybrid model of drug-binding site (“vacuum cleaner”+“flippase”) (T. W. Loo et al., Biochemistry 43 (2004) 12081-12089) rather than an “aqueous pore” model (E. Teodori et al., Current Drug Targets 7 (2006) 896-909). Recently, a 3 D X-ray structure of mouse Pgp (S. G. Aller et al., Science 323 (2009) 1718-1722) revealed an internal cavity of 6000 Å³ with a 30 Å separation of the two nucleotide binding domains. Additionally, it was shown that the binding of cyclic peptide inhibitors into the internal cavity occurs on different binding sites based on hydrophobic, aromatic interactions and stereo-specificity. Both apo and drug-bound Pgp structures have portals open to the cytoplasm and to the inner leaflet of the lipid bilayer for drug entry, confirming the hybrid model hypothesis (T. W. Loo et al., Biochemistry 43 (2004) 12081-12089).

The term “alkyl” (or Alk) means a saturated or unsaturated aliphatic hydrocarbon group which may be straight or branched having 1 to 6 carbon atoms in the chain. “Branched” means that one or lower alkyl groups such as methyl, ethyl or propyl are attached to a linear alkyl chain. <<Lower alkyl>> means 1 to 4 carbon atoms in the chain which may be straight or branched. The alkyl may be substituted with one or more <<alkyl group substituants>> which may be the same or different, and include for instance halo, cycloalkyl, hydroxy (OH), alkoxy, amino (NH₂), acylamino (NHCOAIk), aroylamino (NHCOAr), carboxy (COOH).

The term “alkoxy” refers to an —O-alkyl radical.

The term “cycloalkyl” as employed herein includes saturated cyclic, bicyclic, tricyclic, or polycyclic hydrocarbon groups having 3 to 12 carbons, wherein any ring atom capable of substitution may be substituted by a substituent. Examples of cycloalkyl moieties include, but are not limited to, cyclohexyl and adamantyl.

The term “halo” refers to the atoms of the group 17 of the periodic table (halogens) and includes in particular fluorine, chlorine, bromine, and iodine atom.

The term “aryl” (or Ar) refers to an aromatic monocyclic, bicyclic, or tricyclic hydrocarbon ring system, wherein any ring atom capable of substitution may be substituted by a substituent. Examples of aryl moieties include, but are not limited to, phenyl, naphthyl, and anthracenyl. The term “aryl” also includes “heteroaryl” which refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom capable of substitution may be substituted by a substituent.

The term “heterocyclyl” (or Het) refers to a nonaromatic 5-7 membered monocyclic, ring system having 1-3 heteroatoms, said heteroatoms being selected from O, N, or S (e.g., carbon atoms and 1-3 heteroatoms of N, O, or S), wherein any ring atom capable of substitution may be substituted by a substituent.

The term “substituents” refers to a group “substituted” on an alkyl, heterocyclyl or aryl group at any atom of that group. Suitable substituents include, without limitation, alkyl, alkenyl, alkynyl, alkoxy, halo, hydroxy, cyano, nitro, amino, SO₃H, sulfate, phosphate, perfluoroalkyl, perfluoroalkoxy, methylenedioxy, ethylenedioxy, carboxyl, oxo, thioxo, imino (alkyl, aryl, aralkyl), S(O)ₙ alkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “oxo” refers to an oxygen atom, which forms a carbonyl when attached to carbon, an N-oxide when attached to nitrogen, and a sulfoxide or sulfone when attached to sulfur.

The term “alkenyl” as employed herein includes partially unsaturated, nonaromatic, hydrocarbon groups having 2 to 12 carbons, preferably 2 to 6 carbons.

The compounds herein described may have asymmetric centers. Compounds of the present invention containing an asymmetrically substituted atom may be isolated in optically active or racemic forms. It is well-known in the art how to prepare optically active forms, such as by resolution of racemic forms or by synthesis from optically active starting materials. All chiral, diastereomeric, racemic forms and all geometric isomeric forms of a compound are intended, unless the stereochemistry or the isomeric form is specifically indicated.

“Pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio.

The term “pharmaceutically acceptable salt” refers to salts which retain the biological effectiveness and properties of the compounds of the invention and which are not biologically or otherwise undesirable. In many cases, the compounds of the invention are capable of forming acid and/or base salts by virtue of the presence of amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts see Berge, et al. ((1977) J. Pharm. Sd, vol. 66, 1). The expression “non-toxic pharmaceutically acceptable salts” refers to non-toxic salts formed with nontoxic, pharmaceutically acceptable inorganic or organic acids or inorganic or organic bases. For example, the salts include those derived from inorganic acids such as hydrochloric, hydrobromic, sulfuric, sulfamic, phosphoric, nitric, and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, fumaric, methanesulfonic, and toluenesulfonic acid and the like.

In the context of the invention, the term “treating” or “treatment”, as used herein, means reversing, alleviating, inhibiting the progress of, or preventing the disorder or condition to which such term applies, or one or more symptoms of such disorder or condition.

While it is possible for the compounds of the invention having formula (I) to be administered alone it is preferred to present them as pharmaceutical compositions. The pharmaceutical compositions, both for veterinary and for human use, useful according to the present invention comprise at least one compound having formula (I) as above defined, together with one or more pharmaceutically acceptable carriers and optionally other therapeutic ingredients.

In certain preferred embodiments, active ingredients necessary in combination therapy may be combined in a single pharmaceutical composition for simultaneous administration.

As used herein, the term “pharmaceutically acceptable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use can also be prepared. The preparation can also be emulsified. In particular, the pharmaceutical compositions may be formulated in solid dosage form, for example capsules, tablets, pills, powders, dragees or granules.

The choice of vehicle and the content of active substance in the vehicle are generally determined in accordance with the solubility and chemical properties of the active compound, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

The pharmaceutical compositions can be administered in a suitable formulation to humans and animals by topical or systemic administration, including oral, rectal, nasal, buccal, ocular, sublingual, transdermal, rectal, topical, vaginal, parenteral (including subcutaneous, intra-arterial, intramuscular, intravenous, intradermal, intrathecal and epidural), intracisternal and intraperitoneal. It will be appreciated that the preferred route may vary with for example the condition of the recipient.

The formulations can be prepared in unit dosage form by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Total daily dose of the compounds of the invention administered to a subject in single or divided doses may be in amounts, for example, of from about 0.001 to about 100 mg/kg body weight daily and preferably 0.01 to 10 mg/kg/day. Dosage unit compositions may contain such amounts of such submultiples thereof as may be used to make up the daily dose. It will be understood, however, that the specific dose level for any particular patient will depend upon a variety of factors including the body weight, general health, sex, diet, time and route of administration, rates of absorption and excretion, combination with other drugs and the severity of the particular disease being treated.

According to an advantageous embodiment, the compounds of formula (I) above are used for reversing or inhibiting multidrug resistance in cancer, or in bacterial, fungal or parasitic infections.

Preferably, the present invention relates to the compound of formula (I) as defined above for its use for reversing or inhibiting multidrug resistance in cancer.

According to a particular embodiment, said compounds of formula (I) are administered together with an antitumoral medicine.

In formula (I) as defined above, preferably, (A) is (Ib) as defined above.

Preferably, in formula (I), at least one of R₃, R′₃, R₄ and/or R₅ is other than H.

Preferably, R₁ and R₁′ are each independently selected from H, OC(═O)Ar, or together with the carbon atom to which they are attached form a ═O group.

Preferably, R₃ is H, OR₈ or OC(═O)Ar, most preferably H, 2-oxytetrahydropyranyl or OC(═O)Ar, notably OC(═O)Ph.

Preferably, R₄ is H, OR₈, or OC(═O)Ar. More preferably, R₄ is H, OH, OC(═O)Ph, or 2-oxytetrahydropyranyl.

Preferably, R₅ is H or OR₉, more preferably H or 2-oxytetrahydropyranyl.

Preferably, one of R₆, R₇ is H, or C(═O)R₁₃, notably C(═O)CH₃.

Preferably, R₆ is C(═O)R₁₃, notably C(═O)CH₃

Preferably, R₆, R₇ are selected from H, C(═O)CH₃, 2-oxytetrahydropyranyl, OC(═O)Ph, or OH(OH₃)(CH₂)₂CO₂CH₃.

R₈ is preferably a 5 to 7 membered cycloalkyl group in which one or more ring carbon atoms are replaced by at least one hetero atom —O—. More preferably, R₈ is a 2-tetrahydropyranyl.

Preferably R₃ and/or R₄ are different from H.

The present invention also relates to a compound of formula (II):

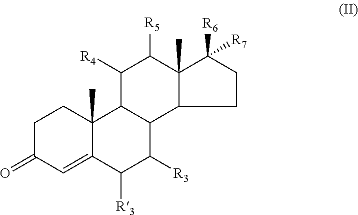

wherein R₃, R′₃, R₄, R₅, R₆ and R₇ are as defined above in formula (I),

for its use for reversing or inhibiting multidrug resistance.

As preferred compounds of formula (II), one may cite the compounds having the following formula (II-1):

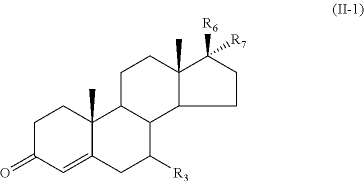

wherein R₃, R₆ and R₇ are as defined above in formula (II).

Preferably, in formula (II) or (II-1), R₃ is H.

According to an advantageous embodiment, in formula (II) or (II-1), R₃ is other than H. Preferably, R₃ is OH or OR₈, R₈ being as defined above and being preferably a heterocyclyl (such as tetrahydropyranyl).

Preferably, in formula (II) or (II-1), R₆ and R₇ are each independently selected from H, COR₁₃, and OR₁₄, R₁₃ being preferably alkyl and R₁₄ being preferably a heterocycle.

The present invention also relates to a compound having the following formula (III′):

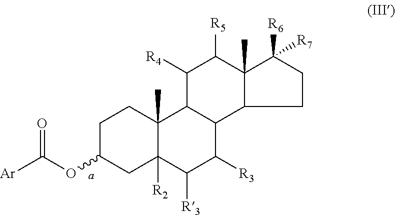

wherein R₂, R₃, R′₃, R₄, R₅, R₆ and R₇ are as defined above in formula (I),

for the use as mentioned above.

It should be mentioned here that the group —OCOAr is either above or below the plane of the molecule. The bond “a” which is represented by [symbol] may thus be either [symbol] or [symbol] .

Preferably, in formula (III′), at least one of R₃, R′₃, R₄ and/or R₅ is other than H.

The present invention also relates to a compound of formula (III):

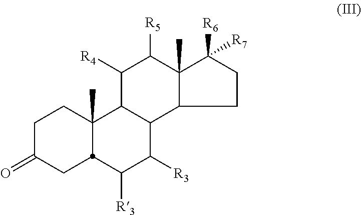

wherein R₃, R′₃, R₄, R₅, R₆ and R₇ are as defined above in formula (I),

for its use for reversing or inhibiting multidrug resistance.

Preferably, in formula (III), at least one of R₃, R′₃, R₄ and/or R₅ is other than H.

Preferably, in formula (III), R₃ is —OCOPh, 2-oxytetrahydropyranyl or OR₈, R₈ being preferably a heterocyclic group.

The dot in the formula (III) indicates the position of H(R₂ of formula (I)). The compounds of formula (III) are 5β-H derivatives.

The present invention also relates to a compound of formula (III-1):

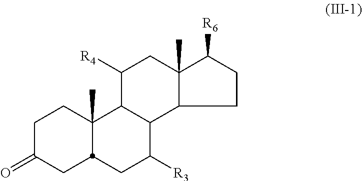

wherein R₃, R₄, and R₆ are as defined above in formula (I),

for its use for reversing or inhibiting multidrug resistance.

Preferably, in formula (III-1), at least one of R₃ and R₄ is other than H.

Preferably, in formula (III-1), R₃ is selected from OR₈ (R₈ being preferably a heterocyclyl group), OCOAr and H.

Preferably, in formula (III-1), R₆ is COR₁₃.

Preferably, in formula (III-1), R₄ is OR₈ (R₈ being in particular a heterocyclyl group).

A preferred group of compounds of the invention are thus consisted by compounds having formula (III-2) as follows:

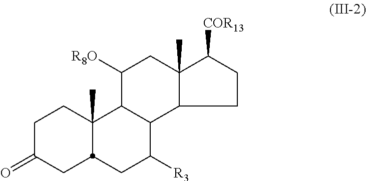

R₃, R₈ and R₁₃ being as defined above in formula (I), and R₃ being preferably selected from OR₈ (R₈ being preferably a heterocyclyl group), OCOAr and H.

A preferred group of compounds of the invention are thus consisted by compounds having formula (III-3) as follows:

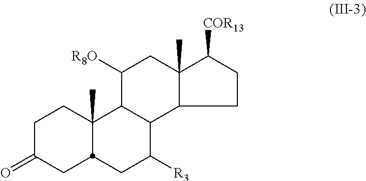

R₃, R₈ and R₁₃ being as defined above in formula (I), and R₃ being preferably selected from OR₈ (R₈ being preferably a heterocyclyl group), OCOAr and H.

The present invention also relates to a compound of formula (IV):

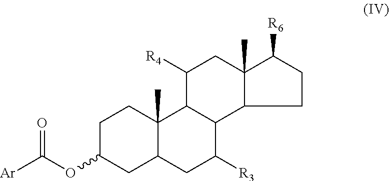

wherein R₃, R₄ and R₆ are as defined above in formula (I),

for its use for reversing or inhibiting multidrug resistance.

Preferably, in formula (IV), at least one of R₃ and R₄ is other than H.

The compounds of formula (IV) are 5α-H derivatives.

Preferably, in formula (IV), R₃ is OCOAr.

Preferably, in formula (IV), R₆ is COR₁₃.

Preferably, in formula (IV), R₄ is H.

The bond [symbol] may be either [symbol] or [symbol] .

A preferred group of compounds of the invention are thus consisted by compounds having formula (IV-1) as follows:

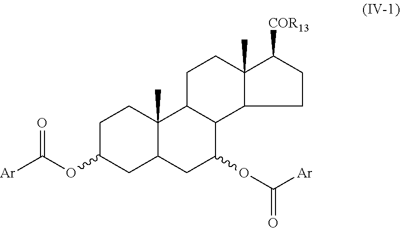

R₁₃ being as defined above in formula (I), and being preferably alkyl.

The present invention also relates to a compound of formula (V):

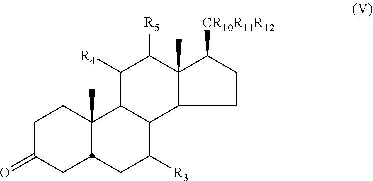

wherein R₃, R₄, R₅, R₁₀, R₁₁ and R₁₂ are as defined above in formula (I),

and one of R₁₀, R₁₁ and R₁₂ groups being (CH₂)_{q}OAlk or (CH₂)_{q}C(═O)OAlk,

for its use for reversing or inhibiting multidrug resistance.

Preferably, in formula (V), at least one of R₃, R₄ and/or R₅ is other than H.

A preferred group of compounds of the invention are thus consisted by compounds having formula (V-1) or (V-2) as follows:

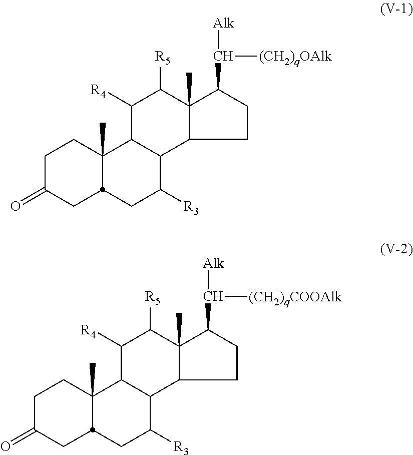

R₃, R₄, and R₅ being as defined above in formula (I).

Preferably, in formula (V-1) or (V-2), at least one of R₃, R₄ and/or R₅ is other than H.

Preferably, in formulae (V-1) and (V-2), R₄ is H.

Preferably, in formulae (V-1) and (V-2), R₃ and R₄ are selected from OR₈ and OCOAr, R₈ being preferably a heterocycle (such as 2-tetrahydropyranyl).

Preferably, in formulae (V-1) and (V-2), R₄ is H, and R₃ and R₄ are selected from OR₈ and OCOAr, R₈ being preferably a heterocycle.

The present invention also relates to a compound of formula (VI):

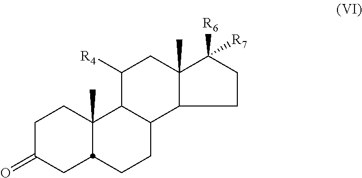

wherein R₄, R₆ and R₇ are as defined above in formula (I),

and wherein R₄, R₆, and R₇ are not H,

for its use for reversing or inhibiting multidrug resistance.

The present invention also relates to a compound of formula (VII):

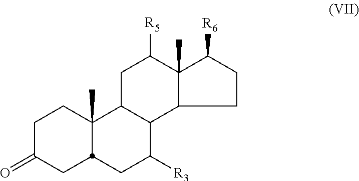

wherein R₃, R₅ and R₆ are as defined above in formula (I), R₅ being not H,

for its use for reversing or inhibiting multidrug resistance.

The present invention also relates to a pharmaceutical composition comprising a compound of formula (I) as defined above, in admixture with one or more pharmaceutically acceptable excipients,

with the exclusion of the compounds of formula (I) having the following formulae:

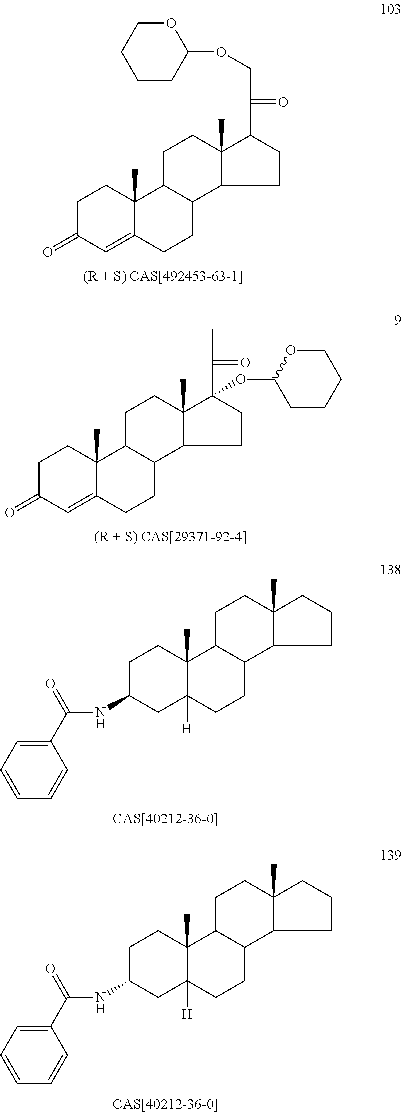

The compounds 103(R+S) CAS[492453-63-1] and 9 (R+S) CAS[29371-92-4] as mentioned above are in the form of racemic mixtures.

The present invention also relates to a pharmaceutical composition comprising a compound of formula (VIII):

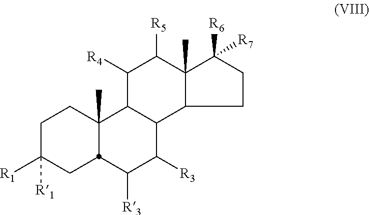

wherein R₁, R′₁, R₃, R′₃, R₄, R₅, R₆ and R₇ are as defined above in formula (I),

in admixture with one or more pharmaceutically acceptable excipients.

Compounds of formula (VIII) are 5β-derivatives.

Preferably, in formula (VIII), at least one of R₃, R′₃, R₄ and/or R₅ is other than H.

The present invention also relates to a pharmaceutical composition comprising a compound of formula (II) as defined above, in admixture with one or more pharmaceutically acceptable excipients, with the exclusion of the compounds 103(R+S) CAS[492453-63-1] and 9 (R+S) CAS[29371-92-4] as mentioned above.

The present invention also relates to a pharmaceutical composition comprising a compound of formula (II-1) as defined above, in admixture with one or more pharmaceutically acceptable excipients, with the exclusion of the compounds 103(R+S) CAS[492453-63-1] and 9 (R+S) CAS[29371-92-4] as mentioned above.

The present invention also relates to a pharmaceutical composition comprising a compound of formula (I) as defined above wherein R₃ is other than H, in admixture with one or more pharmaceutically acceptable excipients.

The present invention also relates to a pharmaceutical composition comprising a compound of formula (IX)

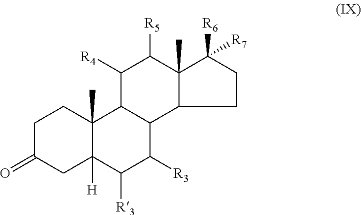

wherein R₃, R′₃, R₄, R₅, R₆ and R₇ are as defined above in formula (I),

in admixture with one or more pharmaceutically acceptable excipients.

In formula (IX), the hydrogen atom in position 5 is either in α- or β-position.

Preferably, in formula (IX), at least one of R₃, R′₃, R₄ and/or R₅ is other than H.

The present invention also relates to a pharmaceutical composition comprising a compound of formula (III′) as defined above, in admixture with one or more pharmaceutically acceptable excipients.

The present invention also relates to a pharmaceutical composition comprising a compound having one of formulae (III), (III-1), (III-2), (IV), (IV-1), (V), (V-1), (V-2), (VI) or (VII), as defined above, in admixture with one or more pharmaceutically acceptable excipients.

The present invention also relates to a compound having formula (II) as defined above, with the exclusion of the R/S mixture of 9 (R+S) CAS[29371-92-4] and 138 CAS[40212-36-0] as mentioned above.

The present invention also relates to compounds having formula (II-1):

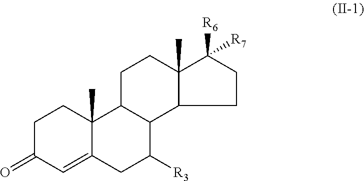

R₃, R₆ and R₇ being as defined above in formula (I),

and R₃ being other than H.

The present invention also relates to compounds having formula (III) as

with the exclusion of the R/S mixture of

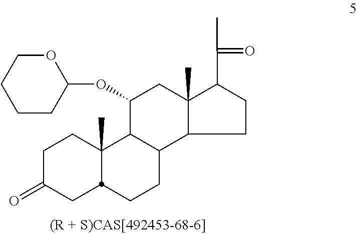

and the exclusion of

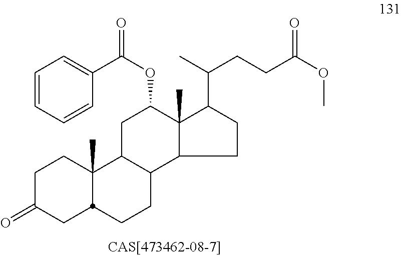

The present invention also relates to compounds having formula (IV) as defined above, with the exclusion of

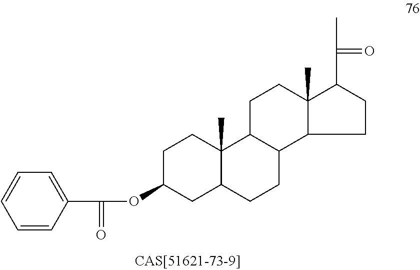

The present invention also relates to compounds having formula (IV-1), (V-1) or (VI) as defined above.

The present invention also relates to compounds having formula (V) or (V-2) as defined above, with the exclusion of

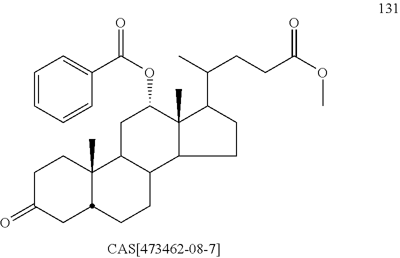

The present invention also relates to compounds having formula (VII) as to defined above, with the exclusion of

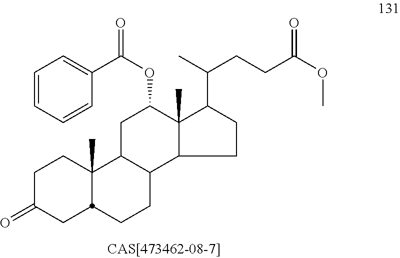

The present invention also relates to the following preferred compounds:

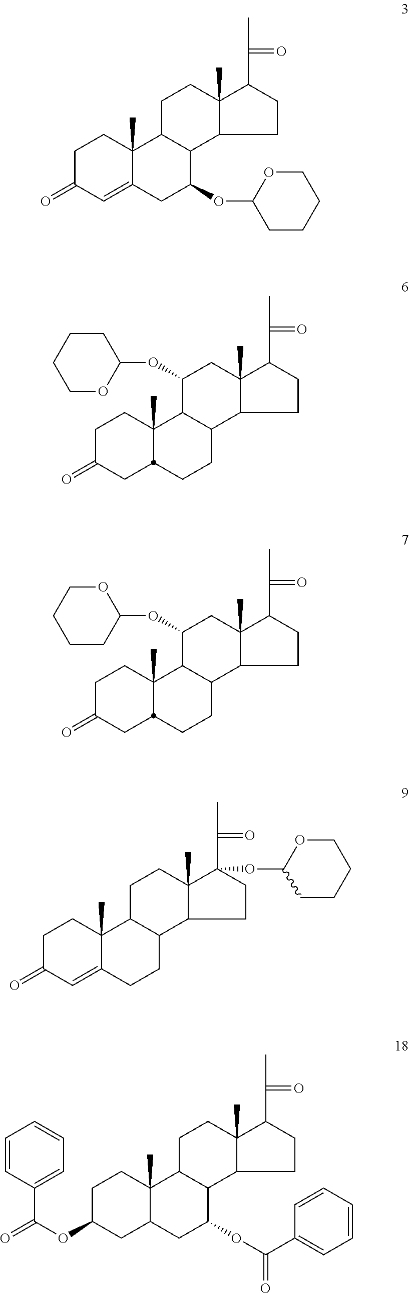

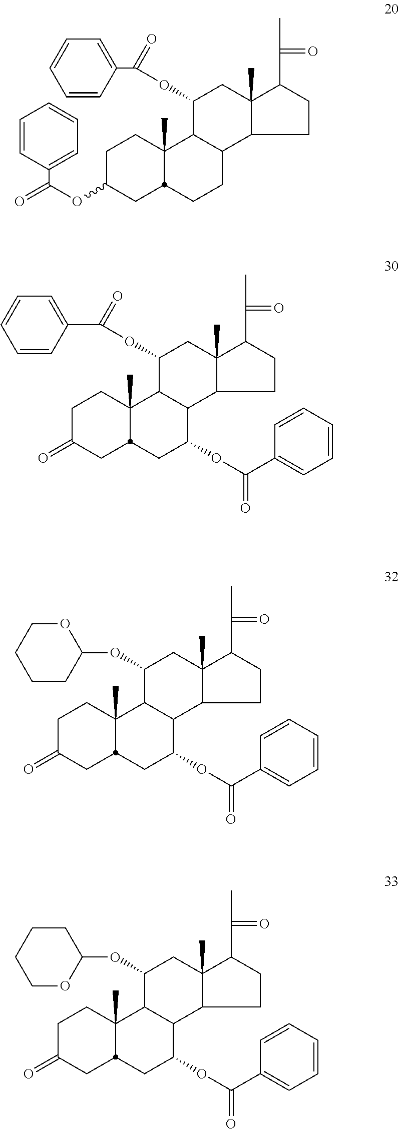

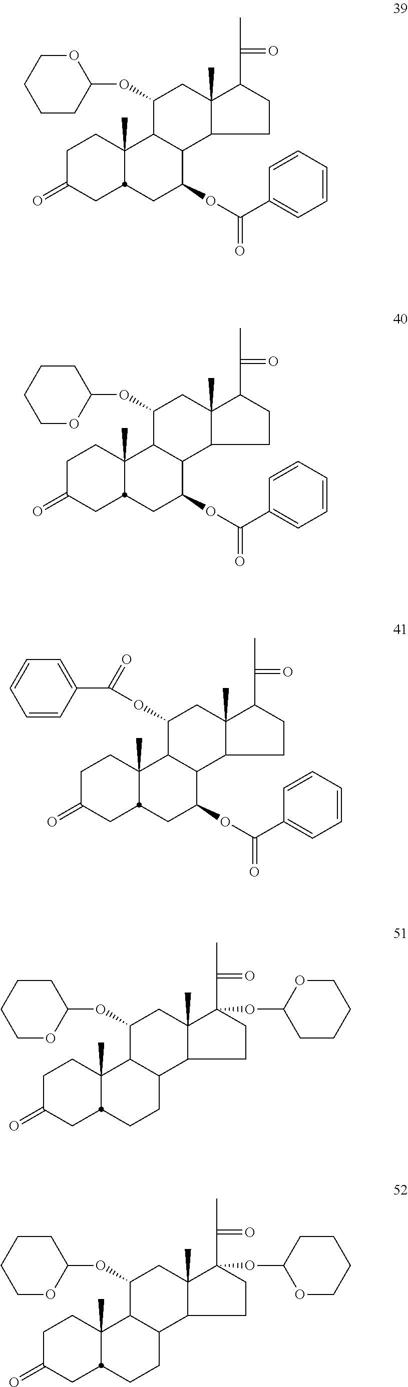

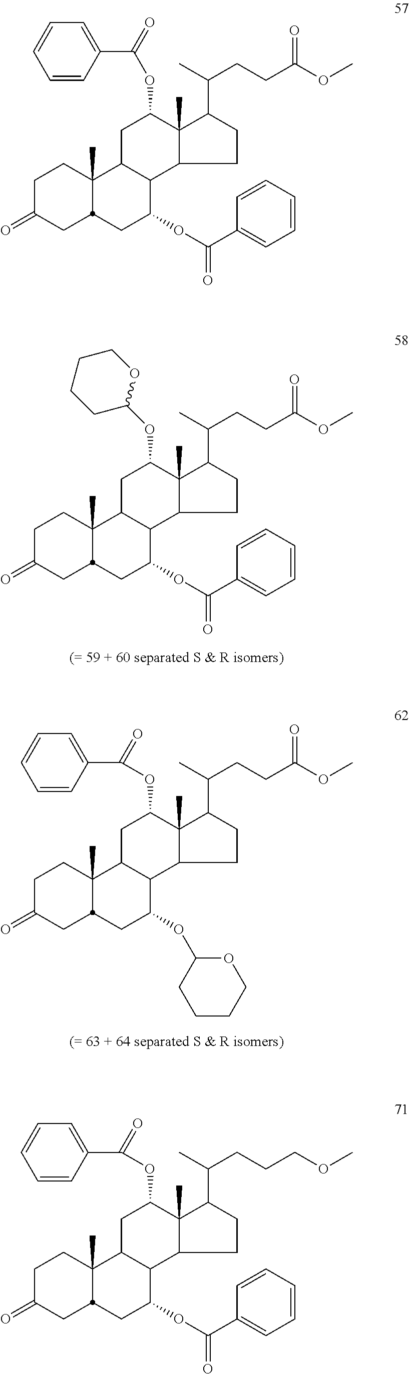

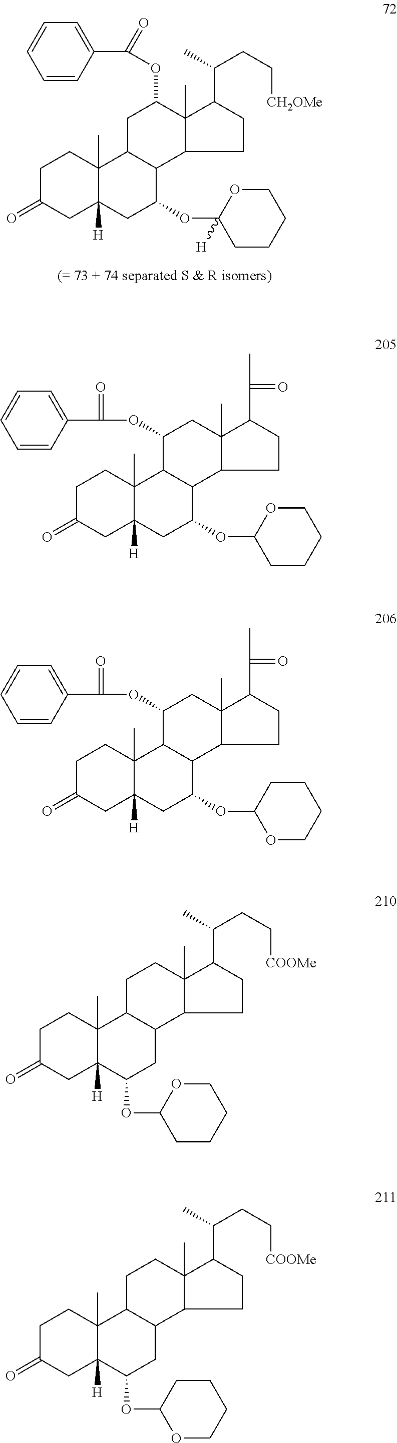

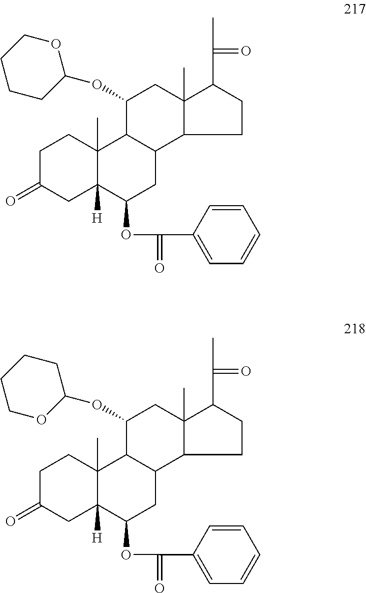

The present invention also relates to a pharmaceutical composition comprising one of said preferred compounds, in association with at least one pharmaceutically acceptable excipient, as well as said compounds for their use for reversing or inhibiting multidrug resistance.

### FIGURES

FIGS. 1 and 2 represent the effects of treatment with different cytotoxics with a concentration varying from 0.001 μM to 100 μM for 24 h on survival of H295R and R7 cells, evaluated by [³H]thymidine incorporation. They represent the ratio of [³H]thymidine incorporation (in %) in surviving cells (1: K562/R7 and 2: H295R cells) as a function of the concentration in μM of several cytotoxic drugs. The curve with black circles relates to doxorubicin; the curve with white circles relates to vinorelbine; the curve with black triangles relates to taxol; the curve with white triangles relates to vinblastine; the curve with the black squares relates to mitoxantrone and the curve with white squares relates to colchicine.

FIGS. 3 and 4 represent the effects of compounds of the invention (steroid modulators) on chemosensitization of resistant R7 cells to doxorubicin (DOXO), evaluated by [³H]thymidine incorporation. Resistant R7 cells were incubated for 24 hours with increasing concentrations of doxorubicinin the absence (∘) or in the presence of said steroid modulators. As a control, the sensitive K562 cells were incubated with the same concentrations of doxorubicin ().

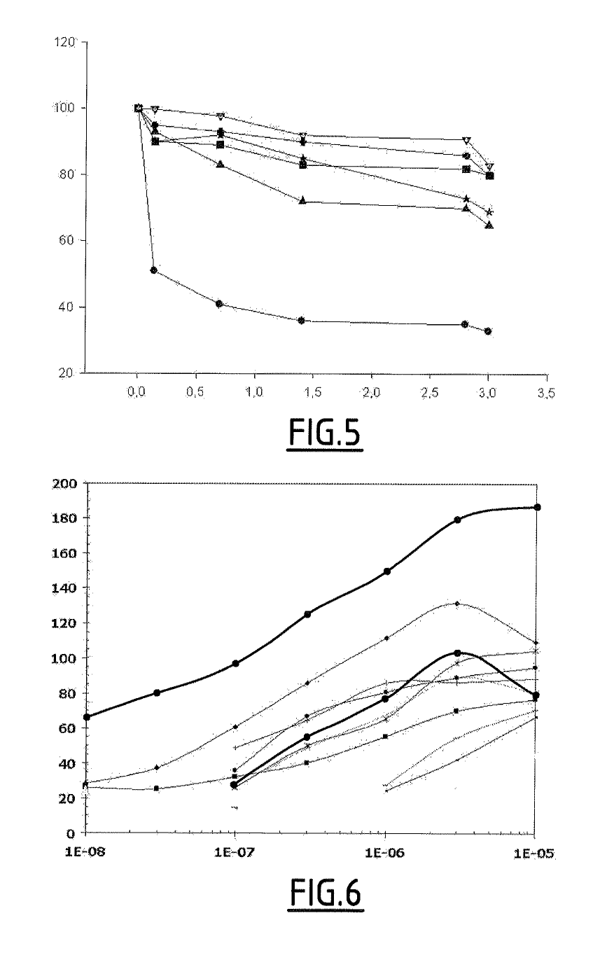

In FIG. 3, the curve with the black triangles (▾) relates to progesterone at 3 μM; the curve with white lozenges (⋄) relates to compound (30) at 0.4 μM; the curve with black squares (▪) relates to compound (33) at 0.4 μM; the curve with white squares (□) relates to compound (41) at 0.5 μM; the curve with black lozenges (♦) relates to compound (51) at 0.5 μM; the curve with black triangles (▴) relates to cyclosporine A at 0.4 μM.

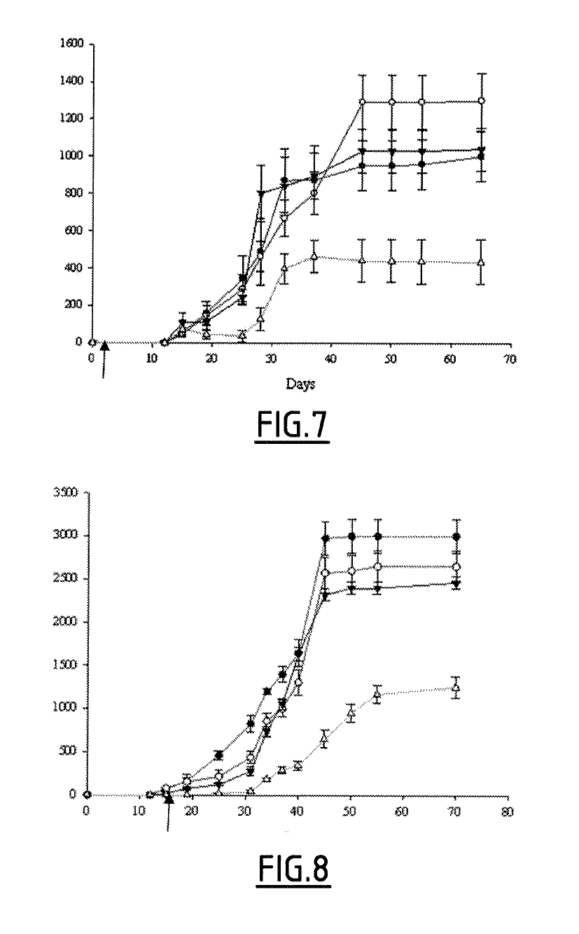

In FIG. 4, the curve the black triangles (▾) relates to compound (73) at 0.5 μM; the curve with black squares (▪) relates to compound (71) at 0.5 μM; and the curve with white triangles (Δ) relates to cyclosporine A at 0.4 μM.

FIG. 5 represents the competitive binding assay of compounds of the invention to progesterone receptor. The ratio B/B° is represented as a function of the concentration of the tested compounds (in μM). The curve with black lozenges (♦) relates to ORG2054; the curve with black squares (▪) relates to compound (30); the curve with black circles () relates to compound (33); the curve with black stars (★) relates to compound (41); the curve with black triangles (▴) relates to compound (53) and the curve with white triangles (Δ) relates to compounds (59)+(62).

FIG. 6 represents the activation of hPXR receptor by compounds of the invention using SR6 (synthetic cholesterol-lowering drug) as a positive control (curve with squares) (▪). The tested compounds are: 10 ( [symbol] ), 11 (▪), 30 (+), 33 (▪), 40 (−), 41 (♦), 51 (x), 52 (▾), 53 (), 59+62 (▴). The luciferase activity (RLU) is represented as a function of the concentration of the compounds in M.

FIGS. 7 and 8 represent the tumour volume of mice treated with vehicle (), doxorubicin (∘), compound (33) alone (▾), and compound (33) with doxorubicin (Δ). The tumor volume is indicated as a function of days. FIG. 7 concerns mice xenografted with H295R cells and FIG. 8 concerns mice xenografted with R7 cells (the arrow indicates the beginning of treatment of mice).

### EXAMPLES

### Chemical Synthesis of Compounds of the Invention

### Example 1

### Preparation of 7-mono-substituted pregn-4-ene-3,20-dione derivative (3)

The compound (3) is prepared according to the following reaction scheme:

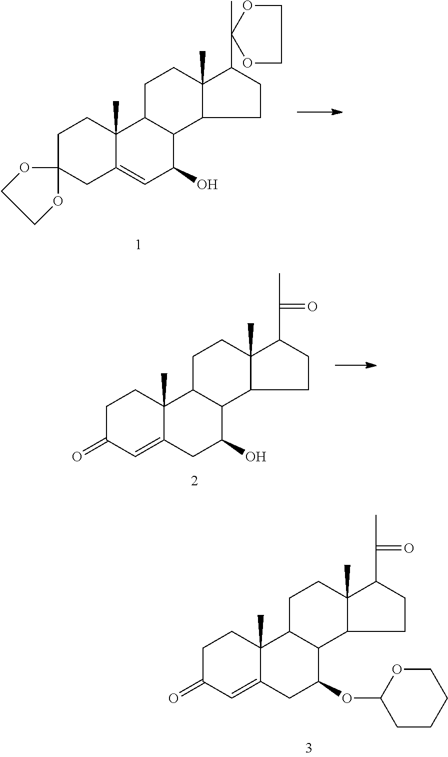

### 3,20-Bis-ethylenedioxy-pregn-5-en-7beta-ol (1)

3,20-Bis-ethylenedioxy-pregn-5-en-7-one (cf Mappus E. & Cuilleron C. Y., J. Chem. Res. 1979 [S], 42-3; [M], 501-35) (100 mg, 0.240 mmol, 1.0 Eq.) dissolved in a mixture of THF (2 mL) and methanol (1 mL) was magnetically stirred under nitrogen for 30 min at 4° C. in the presence of NaBH₄ (15 mg, 0.344 mmol, 1.4 Eq.). The reaction was cooled at 4° C. and the excess of NaBH₄ was neutralized by addition of a mixture of acetone (0.5 mL), acetic acid (0.03 mL) and water (0.5 mL) at 4° C.

After conventional extraction (ethyl acetate) the crude extract was analyzed by TLC on fluorescent silica gel (petroleum ether/ethyl acetate 3:2) which showed two major spots corresponding to 7alpha- and 7beta-ol isomers.

The product was purified by preparative TLC on fluorescent silica gel in similar conditions to give the pure 7beta-ol isomer 1 (55 mg) as the more retained product and the 7alpha-ol isomer (19 mg).

Conventional extraction procedure: the reaction mixture was evaporated either under reduced pressure or under a nitrogen stream for very small volumes, taken up in water and extracted (×3/4 times) with the mentioned organic solvent. The combined organic layers were washed with water (×3/4 times) and dried either by filtration on a phase-separating paper (Whatman) or by addition of solid sodium sulphate. The solvent was evaporated to dryness either under reduced pressure or under a nitrogen stream for very small volumes. When present, residual pyridine was eliminated by azeotropic evaporation in the presence of n-heptane.

### 7beta-Hydroxypregn-4-ene-3,20-dione (2)

A solution of bis-ethyleneketal derivative 1 (50 mg, 0.119 mmol, 1.0 Eq.) in acetone (12 mL) containing p-toluenesulfonic acid monohydrate (20 mg) and a small amount of water (0.6 mL) was magnetically stirred under nitrogen for 3 days at rt. The reaction was quenched with a cold saturated NaHCO₃ solution.

After conventional extraction (ethyl acetate) the crude extract was analyzed by TLC on fluorescent silica gel (petroleum ether/ethyl acetate 1:1).

The product was purified by preparative TLC on fluorescent silica gel in similar conditions (×2 developments) to give a slight amount of UV-absorbing 7beta-hydroxysteroid 2 (6 mg) and a major amount of 4,6-dien-3-one at a much higher Rf value (32 mg).

### 7beta-O-[2′(R+S)]Tetrahydropyranyloxypregn-4-ene-3,20-dione (3)

The 7beta-hydroxy derivative 2 (6 mg, 0.018 mmol, 1.0 Eq.) was stirred for 1 h at room temperature in an extemporaneously prepared solution containing anhydrous THF (0.42 mL), freshly distilled dihydropyrane (0.08 mL) and p-toluenesulfonic acid (0.4 mg). The reaction was stopped by addition of an excess of a saturated NaHCO₃ solution.

After conventional extraction (dichloromethane) the crude extract was analyzed by TLC on fluorescent silica gel (chloroform/methanol 10:1; petroleum ether/ethyl acetate, 1:1) which showed a major UV-absorbing spot at an Rf value different from those of the starting alcohol and the above-mentioned 4,6-dien-3-one.

The product was purified by preparative TLC on fluorescent silica gel (petroleum ether/ethyl acetate 2:1) to give a very low amount of a compound postulated to be a 7beta-O-[2′(R+S)]tetrahydropyranylether derivative 3, based on chromatographic properties.

### Example 2

### Preparation of 11-mono-substituted (5beta-H)pregnane-3,20-dione derivatives (6) and (7)

The compounds (6) and (7) are prepared according to the following reaction

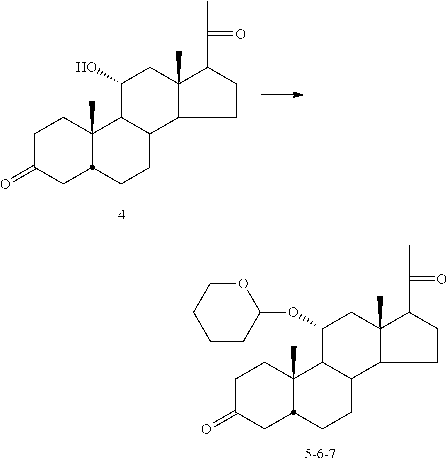

### 11α-Hydroxy(5beta)pregnane-3,20-dione (4)

11α-Hydroxypregn-4-ene-3,20-dione (“11α-hydroxyprogesterone”)(Sigma)(1.0 g, 3.03 mmol, 1.0 Eq.) dissolved in a dioxane-95% ethanol 1:1 v/v mixture containing 0.8% pyridine (82 mL) was introduced in a glass hydrogenation apparatus and magnetically stirred under hydrogen at atmospheric pressure, for 17 h at 30° C., in the presence of 10% Pd—C catalyst (686 mg).

The reaction mixture was filtered on a pad of ™Celite and evaporated under reduced pressure. The residual pyridine was eliminated by azeotropic distillation in the presence of n-heptane.

The residue was analyzed by TLC on fluorescent silica gel (petroleum ether/ethyl acetate 1:3) which showed a major non-UV-absorbing spot.

The product was purified by preparative TLC on fluorescent silica gel in similar conditions to give the pure 4,5-dihydrogenated dione 4 (749 mg).

### 11α-O-[2′(S)]Tetrahydropyranyloxy(5β)pregnane-3,20-dione (6)

11α-O-[2′(R)]Tetrahydropyranyloxy(5β)pregnane-3,20-dione (7)

The 11alpha-hydroxy derivative 4 (100 mg, 0.301 mmol, 1.0 Eq.) was stirred for 1 h at rt° in an extemporaneously prepared solution containing anhydrous THF (2.50 mL), freshly distilled dihydropyrane (0.50 mL) and p-toluenesulfonic acid (2.5 mg). The reaction was stopped by addition of an excess of a saturated NaHCO₃.

After conventional extraction (ethyl acetate) the residue was analyzed by TLC on fluorescent silica gel (petroleum ether/ethyl acetate 2:1).

TLC using petroleum ether/ethyl acetate 2:1 showed two spots with very close Rf values corresponding to the presence of unseparated R/S isomers in the reported tetrahydropyranyl ether derivative 5 [CAS 492453-68-6]. These two isomers were separated by preparative TLC in similar conditions to give pure samples of the less retained isomer 6 (64.4 mg) and of the more retained isomer 7 (50.2 mg).

less retained 11alpha-O-[2′(S)]THP isomer 6

¹H NMR (CDCl₃) 0.652 (s, 18-CH₃), 1.142 (s, 19-CH₃), 2.153 (s, 21-CH₃), 3.5 & 3.9 (2m, 6′-H, 2H), 4.12 (m, 11-H), 4.58 (m, 2′-H).

¹³C NMR (CDCl₃) 39.22 (1), 38.45 (2), 214.62 (3), 42.77 (4), 46.22 (5), 25.86 (6), 26.71 (7), 34.47 (8), 45.10 (9), 36.00 (10), 70.45 (11), 43.68 (12), 43.75 (13), 55.58 (14), 24.47 (15), 23.21 (16), 63.33 (17), 14.41 (18), 22.75 (19), 209.32 (20), 31.58 (21), 96.38 (2′-OTHP), 31.89 (3′-OTHP), 21.71 (4′-OTHP), 25.33 (5′-OTHP), 65.45 (6′-OTHP).

more retained 11alpha-O-[2′(R)]THP isomer 7

¹H NMR (CDCl₃) 0.613 (s, 18-CH₃), 1.120 (s, 19-CH₃), 2.135 (s, 21-CH₃), 3.5 & 3.9 (2m, 6′-H, 2H), 3.80 (m, 11-H), 4.55 (m, 2′-H).

¹³C NMR (CDCl₃) 39.65 (1), 38.34 (2), 213.76 (3), 42.66 (4), 45.91 (5), 26.03 (6), 26.74 (7), 34.81 (8), 45.24 (9), 36.00 (10), 78.48 (11), 47.87 (12), 43.95 (13), 55.19 (14), 24.52 (15), 22.74 (16), 63.51 (17), 14.32 (18), 23.48 (19), 209.09 (20), 31.61 (21), 101.37 (2′-OTHP), 31.30 (3′-OTHP), 20.04 (4′-OTHP), 25.32 (5′-OTHP), 62.99 (6′-OTHP).

### Example 3

### Preparation of 17-mono-substituted pregn-4-ene-3,20-dione derivatives (10) and (11)

This example relates to the preparation of 17alpha-O-[2′(S)]Tetrahydro-pyranyloxypregn-4-ene-3,20-dione 10 and 17alpha-O-[2′(R)]Tetrahydropyranyl-oxypregn-4-ene-3,20-dione 11 having the following formula:

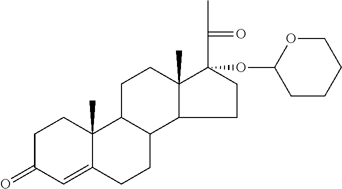

### 17alpha-O-[2′(S)]Tetrahydropyranyloxypregn-4-ene-3,20-dione (10)

### 17alpha-O-[2′(R)]Tetrahydropyranyloxypregn-4-ene-3,20-dione (11)

17alpha-Hydroxypregn-4-ene-3,20-dione (“17alpha-hydroxyprogesterone”) (50 mg, 0.151 mmol, 1.0 Eq.) was stirred for 2 days at rt° in an extemporaneously prepared solution containing anhydrous THF (1.25 mL), freshly distilled dihydropyrane (0.25 mL) and p-toluenesulfonic acid (1.25 mg). Then the same amount of these reagents was further added and stirring was maintained for 5 days until most of the starting product was transformed. The reaction was stopped by addition of an excess of a saturated NaHCO₃ solution.

After conventional extraction (ethyl acetate) the residue was analyzed by TLC on fluorescent silica gel (petroleum ether/ethyl acetate, 2:1). The remaining starting product was eliminated by a first preparative TLC on fluorescent silica gel (petroleum ether/ethyl acetate 2:1). The uncontaminated mixture of the two R/S isomers present in the reported tetrahydropyranylether derivative 9 [CAS 29371-92-4] (49.7 mg) was separated by preparative TLC (dichloromethane/ethyl acetate 20:1, ×6 developments) to give pure samples of the less retained isomer 10 (13.5 mg) and of the more retained isomer 11 (12 mg).

less retained 17alpha-O-[2′(S)]THP isomer 10 (structure confirmed by X-ray crystallographic data)

¹H NMR (CDCl₃) 0.635 (s, 18-CH₃), 1.182 (s, 19-CH₃), 2.189 (s, 21-CH₃), 3.5 & 3.9 (2m, 6′-H, 2H), 4.46 (m, 2′-H), 5.73 (s, 4-H).

¹³C NMR (CDCl₃) 35.64 (1), 33.96 (2), 199.60 (3), 123.84 (4), 171.24 (5), 32.83 (6), 31.96 (7), 35.72 (8), 53.21 (9), 38.55 (10), 20.72 (11), 30.60 (12), 47.24 (13), 50.59 (14), 23.50 (15), 23.48 (16), 94.69 (17), 14.62 (18), 17.38 (19), 209.99 (20), 27.45 (21), 96.22 (2′-OTHP), 31.74 (3′-OTHP), 21.45 (4′-OTHP), 25.18 (5′-OTHP), 65.17 (6′-OTHP).

more retained 17alpha-O-[2′(R)]THP isomer 11 (structure confirmed by X-ray crystallographic data)

¹H NMR (CDCl₃) 0.627 (s, 18-CH₃), 1.186 (s, 19-CH₃), 2.122 (s, 21-CH₃), 3.5 & 3.9 (2m, 6′-H, 2H), 4.41 (m, 2′-H), 5.74 (s, 4-H).

¹³C NMR (CDCl₃) 35.64 (1), 33.96 (2), 199.58 (3), 123.85 (4), 171.28 (5), 32.85 (6), 31.92 (7), 35.84 (8), 53.13 (9), 38.55 (10), 20.79 (11), 30.82 (12), 47.73 (13), 50.99 (14), 23.69 (15), 26.26 (16), 97.49 (17), 14.60 (18), 17.39 (19), 209.80 (20), 27.18 (21), 96.61 (2′-OTHP), 31.84 (3′-OTHP), 20.16 (4′-OTHP), 25.16 (5′-OTHP), 63.33 (6′-OTHP).

### Example 4

### Preparation of 3,7-disubstituted (5α-H)pregnan-20-one derivatives (18)

This example relates to the preparation of 3β,7α-dibenzoyloxy(5alpha)-pregnan-20-one (18), according to the following reactions scheme:

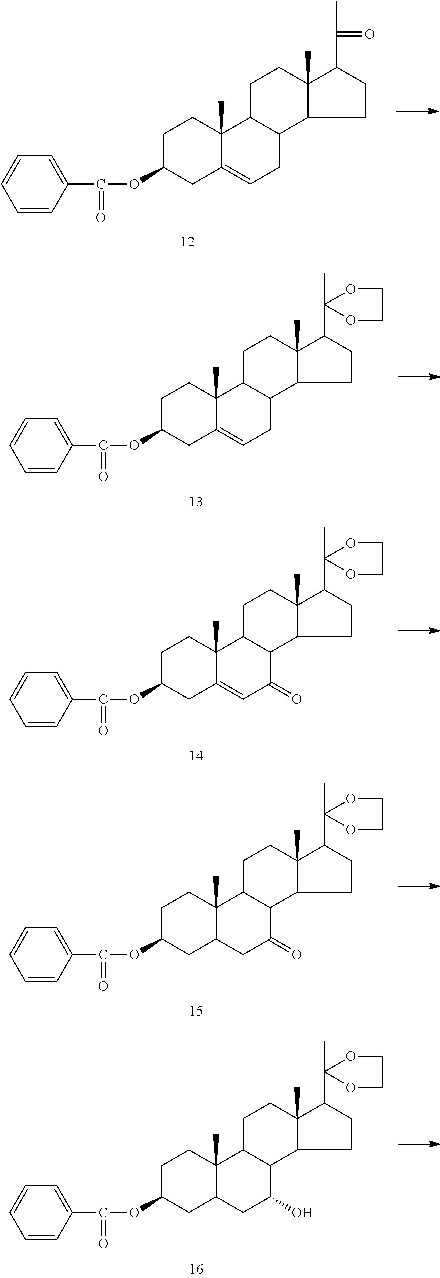

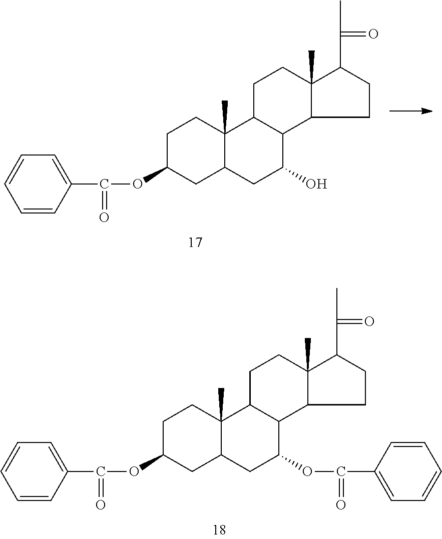

### 3beta-Benzoyloxypregn-5-en-20-one (12)

3beta-Hydroxypregn-5-en-20-one (“pregnenolone”) (10.0 g, 31.598 mmol, 1.0 Eq.) (Sigma) was stirred for 4 h at rt° with benzoyl chloride (10 mL, 86.149 mmol, 2.73 Eq.) dissolved in pyridine (200 mL). The reaction mixture was cooled at 4° C. and stirred for 30 min after addition of 150 mL of ethyl acetate and 150 mL of a saturated aqueous solution of NaHCO₃.

After conventional extraction (ethyl acetate) and elimination of traces of pyridine by azeotropic evaporation in the presence of n-heptane, the crude benzoylated product 12 was analyzed by TLC on fluorescent silica gel (petroleum ether/ethyl acetate 3:1) and was employed without further purification in the following next step.

### 3beta-Benzoyloxypregn-5-en-20-ethyleneketal (13)

A solution of crude 20-ketone 12 (13.4 g, 31.861 mmol, 1.0 Eq.) in toluene (1500 mL) containing ethyleneglycol (150 mL) and pyridinium hydrochloride (1.5 g) was stirred for 24 h under reflux, using a Dean-Stark water trap. The reaction mixture was cooled at 4° C., neutralized with solid NaHCO₃ then with aqueous NaHCO₃.

After conventional extraction (ethyl acetate) the crude 20-ketal 13 was analyzed by TLC on fluorescent silica gel (petroleum ether/ethyl acetate 3:1) and was employed without further purification in the next step.

### 20-Ethylenedioxy-3beta-benzoyloxypregn-5-en-7-one 14

The dry pregn-5-ene derivative 13 (5.0 g, 10.761 mmol, 1.0 Eq.) was added to a vigorously stirred solution of anhydrous CrO₃-(pyridine)₂ complex (69.4 g, 269.025 mmol, 25 Eq.) (Mappus E. & Cuilleron C. Y., J. Chem. Res. 1979 [S], 42-3; [M], 501-35) extemporaneously prepared at 4° C. by addition of anhydrous pyridine and dry CrO₃ in 760 mL of anhydrous dichloromethane. Stirring was maintained for 15 min at 4° C., then for 5 h at rt°.

The reaction mixture was filtered on a column of Florisil® and evaporated under reduced pressure. The residual pyridine was eliminated by azeotropic distillation in the presence of n-heptane. The column was washed with dichloromethane until no more product could be recovered after evaporation.

The crude extract (4.5 g) was analyzed by TLC on fluorescent silica gel (petroleum ether/ethyl acetate 3:1).

The product was purified by flash-chromatography on silica gel (230-400 mesh) using petroleum ether/ethyl acetate 3:1 as eluent to give the pure 5-en-7-one 14 (2.47 g).

### 20-Ethylenedioxy-3beta-benzoyloxy(5alpha)pregnan-7-one (15)

The 5-en-7-one derivative 14 (500 mg, 1.045 mmol, 1.0 Eq.) dissolved in a dioxane-95% ethanol 1:1 v/v mixture containing 0.8% pyridine (42 mL) was introduced in a glass hydrogenation apparatus and magnetically stirred under hydrogen at atmospheric pressure, for 24 h at 30° C., in the presence of 10% Pd—C catalyst (350 mg).

The reaction mixture was filtered on a pad of ™Celite and evaporated under reduced pressure.

The residual pyridine was eliminated by azeotropic distillation in the presence of n-heptane. The crude extract was analyzed by TLC on fluorescent silica gel (petroleum ether/ethyl acetate 3:1) which showed a major non-UV-absorbing spot. This 5,6-dihydrogenated product 15 was employed without further purification in the following next step.

### 20-Ethylenedioxy-3beta-benzoyloxy(5alpha)pregnan-7alpha-ol (16)

To a solution of (5alpha)pregnan-7-one derivative 15 (399 mg, 0.830 mmol, 1.0 Eq.) in anhydrous THF (18 mL) at −78° C. was added 3.05 mL (3.67 Eq.) of a 1 M solution of lithium tri-sec-butylborohydride (L-Selectride, Aldrich) in THF (cf Amann A, Ourisson G, Luu B Synthesis 1987, 1002-4). The solution was stirred under nitrogen for 1.5 h at −78° C. The reaction mixture was stirred for 30 min with 3.05 mL of a 7 N aqueous solution of NaOH and 3.05 mL of 30% of H₂O₂, evaporated under reduced pressure.

After conventional extraction (MTBE) the 7alpha-ol isomer 16 (365 mg) was analyzed by TLC on fluorescent silica gel (petroleum ether/ethyl acetate 3:1).

### 7alpha-Hydroxy-3beta-benzoyloxy(5alpha)pregnan-20-one (17)

A solution of 20-ethylenedioxy-7alpha-ol derivative 16 (330 mg, 0.684 mmol, 1.0 Eq.) in acetone (33.3 mL) containing p-toluenesulfonic acid monohydrate (55 mg) and a small amount of water (1.7 mL) was stirred under nitrogen for 3 h at rt°. The reaction was quenched with a cold saturated NaHCO₃ solution.

After conventional extraction (ethyl acetate) the crude 20-oxo-7alpha-ol derivative 17 was analyzed by TLC on fluorescent silica gel (petroleum ether/ethyl acetate 2:1) and was employed without further purification in the following next step.

### 3beta,7alpha-Dibenzoyloxy(5alpha)pregnan-20-one (18)

The 7alpha-hydroxy derivative 17 (50 mg, 0.114 mmol, 1.0 Eq.) was stirred for 48 h at rt° with benzoyl chloride (0.037 ml, 2.8 Eq.) dissolved in pyridine (0.72 mL). The reaction mixture was cooled at 4° C., stirred for 30 min after addition of 0.8 mL of ethyl acetate and 0.8 mL of a saturated aqueous solution of NaHCO₃.

After conventional extraction (ethyl acetate) and elimination of traces of pyridine by azeotropic evaporation in the presence of n-heptane, the crude extract (60 mg) was analyzed by TLC on fluorescent silica gel (petroleum ether/ethyl acetate 4:1).

The product was purified by preparative TLC on fluorescent silica gel in similar conditions to give the pure dibenzoate 18 (49 mg).

¹H NMR (CDCl₃) 0.681 (s, 18-CH₃), 0.954 (s, 19-CH₃), 2.108 (s, 21-CH₃), 4.96 (m, 3-H), 5.21 (m, 11-H), 7.43 (m, m-Ar—H/3, 2H), 7.55 (m, m-Ar—H/7, 2H+p-Ar—H/3, 1H), 7.61 (m, p-Ar—H/7, 1H), 8.01 (o-Ar—H/7, 2H), 8.04 (o-Ar—H/3, 2H).

¹³C NMR (CDCl₃) 36.60 (1), 27.45 (2), 73.77 (3), 33.41° (4), 47.43 (5), 33.32 (6), 71.48 (7), 38.28 (8), 38.65 (9), 35.64 (10), 21.11 (11), 38.47 (12), 44.22 (13), 50.92 (14), 23.86 (15), 22.80 (16), 63.48 (17), 11.40 (18), 13.16 (19), 208.70 (20), 31.52 (21), 129.52 (o-Ar/3), 128.26 (m-Ar), 132.76 (p-Ar), 130.76 (subst-Ar), 166.06 (Ar—CO), 129.58 (o-Ar/7), 128.52 (m-Ar), 132.99 (p-Ar), 130.72 (subst-Ar), 165.69 (Ar—CO).

### Example 5

### Preparation of 3,11-disubstituted (5beta-H)pregnan-20-one derivatives (20)

This example relates to the preparation of 3alpha/beta,11alpha-dibenzoyloxy (5beta)pregnan-20-one (20).

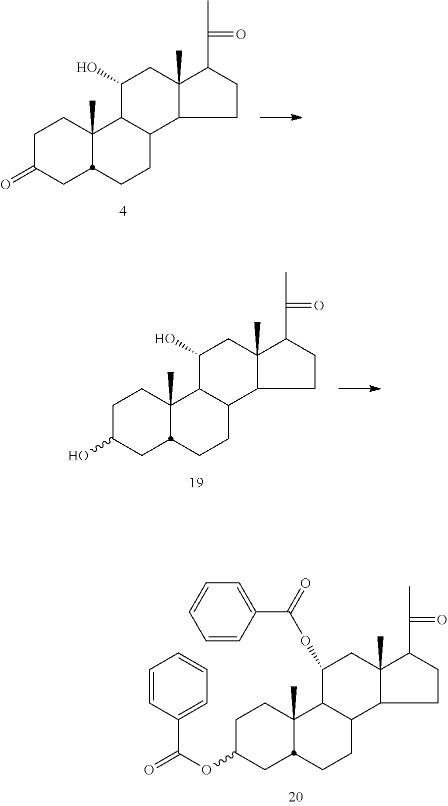

### 3alpha/beta,11alpha-Dihydroxy(5beta)pregnan-20-one (19)

11alpha-Hydroxy(5beta)pregnane-3,20-dione 4 (200 mg, 0.602 mmol, 1.0 Eq.) was magnetically stirred for 48 h at 4° C. with an excess of NaBH₄ (144 mg, 3.806 mmol, 6.32 Eq., added in two equal parts at t=0 and 24 h) in pyridine (4 mL). The reaction was cooled at 4° C. and the excess of NaBH₄ was neutralized by addition of a mixture of acetone (0.5 mL), acetic acid (0.03 mL) and water (0.5 mL) at 4° C.

After conventional extraction (ethyl acetate) and elimination of traces of pyridine by azeotropic evaporation in the presence of n-heptane, the crude extract was analyzed by TLC on fluorescent silica gel (petroleum ether/ethyl acetate 1:3, 1:5 and ethyl acetate) which showed the presence of triol by-products contaminating the expected diol mixture. This crude dihydroxy compound 19 was employed without further purification in the following next step.

### 3alpha/beta,11alpha-Di benzoyloxy(5beta)pregnan-20-one (20)

Crude 3alpha/beta,11alpha-dihydroxy(5beta)pregnan-20-one 19 (50 mg, 0.149 mmol, 1.0 Eq.) was stirred for 45 min at rt° with benzoyl chloride (0.1 mL, 0.861 mmol, 5.76 Eq.) dissolved in pyridine (0.7 mL). The reaction mixture was cooled at 4° C. and stirred for 30 min after addition of 1 mL of ethyl acetate and 1 mL of a saturated aqueous solution of NaHCO₃.

After conventional extraction (ethyl acetate) and elimination of traces of pyridine by azeotropic evaporation in the presence of n-heptane, the crude extract was analyzed by TLC on fluorescent silica gel (petroleum ether/ethyl acetate 3:2 and 3:1).

The product was purified by preparative TLC on fluorescent silica gel. The contaminating tribenzoate by-products were eliminated by a first preparative TLC on fluorescent silica gel (petroleum ether/ethyl acetate 4:1). Then preparative TLC (petroleum ether/ethyl acetate 4:1, ×1 development, then petroleum ether/ethyl acetate 3:1, ×1 development) afforded a pure sample of dibenzoate 20 (17.6 mg) as an unseparated mixture of 3-isomers.

isomer 3alpha (tentative assignments made on the unseparated mixture)

¹H NMR (CDCl₃) 0.758 (s, 18-CH₃), 1.097 (s, 19-CH₃), 2.088 (s, 21-CH₃), 5.00 (m, 3-H), 5.45 (m, 11-H), 7.38 (m, m-Ar—H, 4H), 7.61 (m, p-Ar—H, 2H), 7.97-8.11 (o-Ar—H, 4H).

¹³C NMR (CDCl₃) 37.62 (1), 27.57 (2), 74.82 (3), 32.83 (4), 44.13 (5), 27.12 (6), 26.10 (7), 34.90 (8), 43.41 (9), 35.75 (10), 72.21 (11), 45.73 (12), 44.05 (13), 55.74 (14), 24.34 (15), 22.84 (16), 63.36 (17), 14.29 (18), 23.62 (19), 208.67 (20), 31.48 (21), 129.50 (o-Ar/3alpha), 128.46° (m-Ar), 133.06 (p-Ar), 130.46 (subst-Ar), 166.12 (Ar—CO), 129.56 (o-Ar/11alpha), 128.32° (m-Ar), 132.81 (p-Ar), 130.87 (subst-Ar), 165.70 (Ar—CO).

isomer 3beta (tentative assignments made on the unseparated mixture)

¹H NMR (CDCl₃) 0.739 (s, 18-CH₃), 1.019 (s, 19-CH₃), 2.088 (s, 21-CH₃), 4.90 (m, 3-H), 5.45 (m, 11-H), 7.38 (m, m-Ar—H, 4H), 7.61 (m, p-Ar—H, 2H), 7.97-8.11 (o-Ar—H, 4H).

¹³C NMR (CDCl₃) 37.86 (1), 27.84 (2), 73.62 (3), 32.02 (4), 44.70 (5), 27.12 (6), 26.10 (7), 34.75 (8), 43.41 (9), 35.75 (10), 71.72 (11), 45.43 (12), 43.95 (13), 55.41 (14), 24.34 (15), 22.84 (16), 63.30 (17), 14.15 (18), 23.62 (19), 208.74 (20), 31.53 (21), 129.59 (o-Ar/3beta), 128.24 (m-Ar), 132.73 (p-Ar), 130.59 (subst-Ar), 166.15 (Ar—CO), 129.56 (o-Ar/11alpha), 128.32 (m-Ar), 132.81 (p-Ar), 130.73 (subst-Ar), 165.70 (Ar—CO).

### Example 6

### Preparation of 7,11-disubstituted (5beta-H)pregnane-3,20-dione derivatives (30), (32), (33), (39), (40) and (41)

### Example 6.1

### Preparation of 7alpha,11alpha-Dibenzoyloxy(5beta)pregnane-3,20-dione (30)

The compound (30) is prepared according to the following reaction scheme:

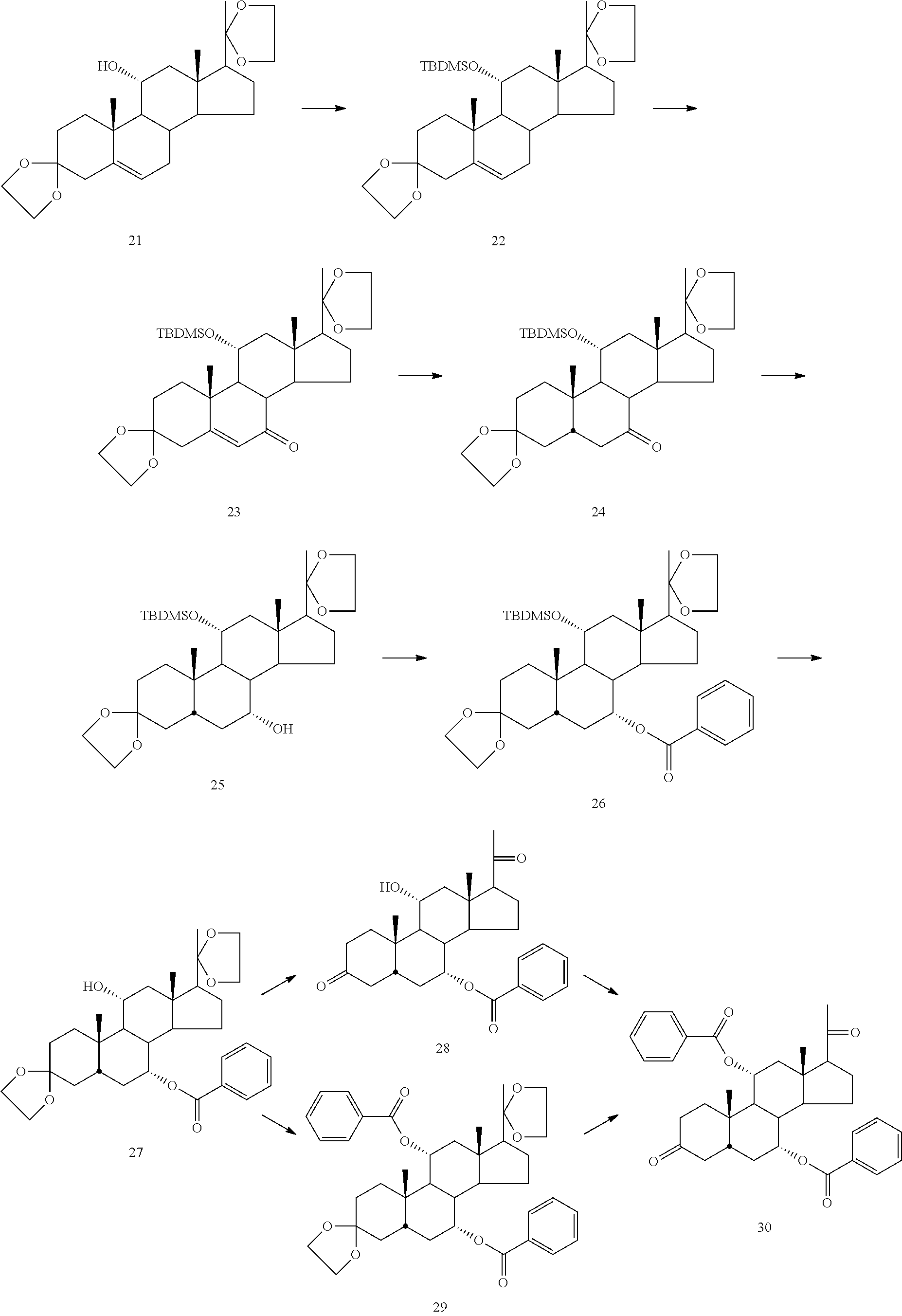

### 3,20-Bis-ethylenedioxypregn-5-en-11α-ol (21)

A solution of 11 alpha-hydroxypregn-4-ene-3,20-dione (“11alpha-hydroxyprogesterone”) (Sigma) (10 g, 30.261 mmol, 1.0 Eq.) in toluene (450 mL) containing ethyleneglycol (140 mL) and p-toluenesulfonic acid (0.6 g) was stirred for 5 h under reflux, using a Dean-Stark water trap. The reaction mixture was cooled at 4° C., neutralized with solid NaHCO₃ then with aqueous NaHCO₃.

After conventional extraction (ethyl acetate) the extract was analyzed by TLC on silica gel (chloroform/ethyl acetate 3:1; chloroform/methanol 10:1).

The crude residue was recrystallized three times from a dichloromethane-methanol mixture containing 1% pyridine to give the pure ethyleneketal derivative. Mother liquors were purified by flash-chromatography on silica gel (230-400 mesh) using chloroform/ethyl acetate 10:1 as eluent. The purified bis-ethyleneketal 21 was dried by addition and evaporation of toluene.

### 11α-(tert-Butyl-dimethyl-silanyloxy)pregn-5-en-3,20-bis-ethyleneketal (22)

A solution of 11alpha-hydroxy derivative 21 (6.0 g, 14.335 mmol, 1.0 Eq.) in anhydrous DMF (240 mL) containing 1H-imidazole (2.37 g, 34.812 mmol, 2.43 Eq.) was cooled at −10° C. (ice-acetone bath). Solid tert-butyldimethylsilyl chloride (5.2 g, 34.498 mmol, 2.40 Eq.) was added under argon atmosphere. The reaction was stirred at rt° for 11 days until completion (monitoring by TLC).

The reaction mixture was extracted by pouring it in 800 mL of water. The solid precipitate of steroid derivative was collected by filtration and washed with water. The solid residue was extracted with ethyl acetate. The organic layer was washed with water, filtered on a phase-separating paper (Whatman), evaporated to dryness under reduced pressure and dried by azeotropic distillation with toluene under reduced pressure.

The dry product (8.55 g) was analyzed by TLC on fluorescent silica gel (petroleum ether/ethyl acetate 5:1).

The crude residue was purified by flash-chromatography on silica gel (230-400 mesh) using petroleum ether/ethyl acetate 6:1 as eluent to give a pure sample of silyl ether 22 (2.4 g) whereas less pure other fractions were recycled.

### 3,20-Bis-ethylenedioxy-11alpha-tert-butyldimethylsilanyloxypregn-5-en-7-one (23)

The dry pregn-5-ene derivative 22 (3.50 g, 6.569 mmol, 1.0 Eq.) was stirred for 15 min at 4° C., then 5 h at rt° in a solution of anhydrous CrO₃-(pyridine)₂ complex (42.368 g, 164.217 mmol, 25 Eq.), extemporaneously prepared at 4° C. by addition of anhydrous pyridine and dry CrO₃ in 400 mL of anhydrous dichloromethane.

The reaction mixture was filtered on a column of ™Florisil and evaporated under reduced pressure. The residual pyridine was eliminated by azeotropic distillation in the presence of n-heptane. The column was washed with dichloromethane until no more product could be recovered after evaporation.

The residue was analyzed by TLC on fluorescent silica gel (petroleum ether/ethyl acetate 2:1).

The crude product was purified by flash-chromatography on silica gel (230-400 mesh) using petroleum ether/ethyl acetate 3:1 as eluent to give the 5-en-7-one 23 (1.02 g).

### 3,20-Bis-ethylenedioxy-11α-tert-butyldimethylsilanyloxy(5beta)pregnan-7-one (24)

The pregn-5-en-7-one 23 (1.576 g, 2.882 mmol, 1.0 Eq.) dissolved in a dioxane-95% ethanol 1:1 v/v mixture containing 0.8% pyridine (124 mL) was introduced in a glass hydrogenation apparatus and magnetically stirred under hydrogen at atmospheric pressure, for 24 h at 30° C., in the presence of 10% Pd—C catalyst (1.11 g).

The reaction mixture was filtered on a pad of ™Celite and evaporated under reduced pressure. The residual pyridine was eliminated by azeotropic distillation in the presence of n-heptane.

The dry 5,6-dihydrogenated product 24 (1.533 g) was analyzed by TLC on fluorescent silica gel (petroleum ether/ethyl acetate 3:1) which showed a major non-UV-absorbing spot.

### 3,20-Bis-ethylenedioxy-11α-tert-butyldimethylsilanyloxy(5beta)pregnan-7alpha-ol (25)

Method A:

To a solution of (5beta)pregnan-7-one derivative 24 (990 mg, 1.804 mmol, 1.0 Eq.) in anhydrous THF (40 mL) at −78° C. was added 6.67 mL (3.7 Eq.) of a 1 M solution of lithium tri-sec-butylborohydride (L-Selectride, Aldrich) in THF (Amann A, Ourisson G, Luu B Synthesis 1987, 1002-4). The solution was stirred under nitrogen for 1.5 h at −78° C. The reaction mixture was stirred for 30 min with 6.67 mL of a 7 N aqueous solution of NaOH and 6.67 mL of 30% H₂O₂, evaporated under reduced pressure.

After conventional extraction (ethyl acetate) the dry product (999 mg) was analyzed by TLC on fluorescent silica gel (petroleum ether/ethyl acetate 2:1, 3:1 and 1:1) which showed two spots corresponding to 7alpha- and 7beta-hydroxy isomers.

The crude residue was purified by flash-chromatography on silica gel (230-400 mesh) using petroleum ether/ethyl acetate 3:1 as eluent to give a pure sample of 7alpha-ol 25 (419 mg), the less retained isomer, and a sample of 7beta-ol by-product 34 (vide infra) (452 mg) still containing a minor amount of the 7alpha-ol.

Method B:

The (5beta)pregnan-7-one derivative 24 (3.64 g, 6.632 mmol, 1.0 Eq.) dissolved in a mixture of THF (15 mL) and methanol (75 mL) was magnetically stirred under nitrogen for 48 h at rt° in the presence of NaBH₄ (300 mg, 7.93 mmol, 1.2 Eq.). The reaction was cooled at 4° C. and the excess of NaBH₄ was neutralized by addition of a mixture of acetone (10 mL), acetic acid (0.5 mL) and water (10 mL) at 4° C.

After conventional extraction (MTBE) the crude extract was analyzed by TLC on fluorescent silica gel (petroleum ether/ethyl acetate 2:1) which showed two major spots.

The crude product was purified by flash-chromatography on silica gel (230-400 mesh) using petroleum ether/ethyl acetate 3:1 as eluent to give the pure 7alpha-ol isomer 25 (1.087 g) as the less retained isomer, an intermediate fraction containing both isomers (1.818 g) and the pure 7beta-ol isomer (0.886 g)

### 7alpha-Benzoyloxy-11alpha-tert-butyldimethylsilanyloxy(5beta)pregnan-3,20-bis-ethyleneketal (26)

The 7alpha-hydroxy derivative 25 (400 mg, 0.726 mmol, 1.0 Eq.) was stirred for 72 h at rt° with benzoyl chloride (0.5 mL, 4.307 mmol, 5.93 Eq.) dissolved in pyridine (8 mL). The reaction mixture was cooled in an ice-bath at 4° C. and stirred for 30 min after addition of 10 mL of ethyl acetate and 10 mL of a saturated aqueous solution of NaHCO₃.

After conventional extraction (ethyl acetate) and elimination of traces of pyridine by azeotropic evaporation in the presence of n-heptane, the extract (491 mg) was analyzed by TLC on fluorescent silica gel (petroleum ether/ethyl acetate 5:1).

The crude product was purified by preparative TLC on fluorescent silica gel (petroleum ether/ethyl acetate 5:1, ×2 developments) to give the 7alpha-benzoate 26.

### 3,20-Bis-ethylenedioxy-7alpha-benzoyloxy(5beta)pregnan-11alpha-ol (27)

The 11alpha-tert-butyldimethylsilyl derivative 26 (C654.95; 1.50 g, 2.290 mmol, 1.0 Eq.) dissolved in THF (17 mL) was stirred for 12 h at 4° C. after addition of a commercial 1 M solution of tetra-butylammonium fluoride (14 mL, 14 mmol, 3.60 g, 6.02 Eq.) in THF.

After conventional extraction (ethyl acetate) the extract (1.37 g) was analyzed by TLC on fluorescent silica gel (petroleum ether/ethyl acetate 1:1).

The crude product was purified by flash-chromatography on silica gel (230-400 mesh) using petroleum ether/ethyl acetate 2:1 as eluent to give a pure sample of 11alpha-ol 27 (0.93 g). The combined less-pure fractions were purified by preparative TLC on fluorescent silica gel (petroleum ether/ethyl acetate 1:1) to give additional amounts of pure 11alpha-ol.

### 7alpha-Benzoyloxy-11alpha-hydroxy(5beta)pregnan-3,20-dione (28)

A solution of bis-ethylenedioxy-11alpha-ol derivative 27 (70 mg, 0.129 mmol, 1.0 Eq.) in acetone (12 mL) containing p-toluenesulfonic acid monohydrate (21 mg) and small amount of water (0.6 mL) was stirred for 2 days at rt°. The reaction was quenched with a cold saturated NaHCO₃ solution.

After conventional extraction (ethyl acetate) the dry product (44 mg) was analyzed by TLC on fluorescent silica gel (petroleum ether/ethyl acetate 1:2).

The crude product was purified by preparative TLC on fluorescent silica gel (petroleum ether/ethyl acetate 1:2) to give the pure 3,20-dione 28 (29 mg).

### 7alpha,11alpha-Di benzoyloxy(5beta)preg nan-3,20-bis-ethyleneketal (29)

The bis-ethylenedioxy-7α-benzoyloxy(5beta)pregnan-11α-ol derivative 27 (69 mg, 0.128 mmol, 1.0 Eq.) was stirred for 12 h at rt° with benzoyl chloride (0.088 mL, 0.758 mmol, 5.94 Eq.) dissolved in pyridine (1.42 mL). The reaction mixture was cooled at 4° C., stirred for 30 min after addition of 5 mL of ethyl acetate and 5 mL of a saturated aqueous solution of NaHCO₃.

After conventional extraction (ethyl acetate) and elimination of traces of pyridine by azeotropic evaporation in the presence of n-heptane, the dry bis-ethylenedioxy-dibenzoate 29 (82 mg recovered) was analyzed by TLC on fluorescent silica gel (petroleum ether/ethyl acetate 2:1 and 1:1).

### 7alpha,11alpha-Di benzoyloxy(5beta)pregnane-3,20-dione (30)

Method A:

A solution of bis-ethylenedioxy-dibenzoate 29 (80 mg, 0.124 mmol, 1.0 Eq.) in acetone (38 mL) containing p-toluenesulfonic acid monohydrate (64 mg) and a small amount of water (1.9 mL) was stirred for 12 h at rt°. The reaction was quenched with a cold saturated NaHCO₃ solution.

After conventional extraction (ethyl acetate) the dry product (68 mg) was analyzed by TLC on fluorescent silica gel (petroleum ether/ethyl acetate 2:1) which showed the presence of a more polar minor mono-dioxolanated by-product.

The crude product was purified by preparative TLC on fluorescent silica gel (petroleum ether/ethyl acetate 2:1) to give a pure sample of dibenzoate 30 (50 mg).

Method B:

7alpha-Benzoyloxy-11alpha-hydroxy(5beta)pregnan-3,20-dione 28 (245 mg, 0.541 mmol, 1.0 Eq.) was stirred for 12 h at rt° with benzoyl chloride (0.35 mL, 3.015 mmol, 5.57 Eq.) dissolved in pyridine (6 mL). The reaction mixture was cooled at 4° C. and stirred for 30 min after addition of 24 mL of ethyl acetate and 24 mL of a saturated aqueous solution of NaHCO₃.

After conventional extraction (ethyl acetate) and elimination of traces of pyridine by azeotropic evaporation in the presence of n-heptane, the crude extract (310 mg) was analyzed by TLC on fluorescent silica gel (petroleum ether/ethyl acetate 2:1).

The crude product was purified by preparative TLC on fluorescent silica gel (petroleum ether/ethyl acetate 2:1, ×2 developments) to give the dibenzoate 30 (230 mg).

¹H NMR (CDCl₃) 0.820 (s, 18-CH₃), 1.235 (s, 19-CH₃), 2.092 (s, 21-CH₃), 5.30 (m, 7-H), 5.64 (m, 11-H), 7.45 (m, m-Ar—H, 4H), 7.58 (m, p-Ar—H, 2H), 8.02 (m, o-Ar—H, 4H).

¹³C NMR (CDCl₃) 38.80 (1), 37.94 (2), 211.78 (3), 45.03 (4), 43.46 (5), 31.04 (6), 71.68 (7), 37.59 (8), 39.46 (9), 36.13 (10), 71.25 (11), 45.19 (12), 43.78 (13), 50.53 (14), 23.68 (15), 22.90 (16), 62.88 (17), 14.03 (18), 22.55 (19), 208.22 (20), 31.47 (21), 129.44/54 (o-Ar/7+11), 128.59/77 (m-Ar/7+11), 133.39/42 (p-Ar/7+11), 130.02/06 (subst-Ar/7+11), 165.43/68 (Ar—CO/7+11).

### Example 6.2

### Preparation of 7α-Benzoyloxy-11α-O-[2′(S)]tetrahydropyranyloxy (5β)pregnane-3,20-dione (32) and 7α-Benzoyloxy-11α-O-[2′(R)]tetrahydropyranyl-oxy(5β)pregnane-3,20-dione (33)

The compounds (32) and (33) are prepared according to the following reaction scheme:

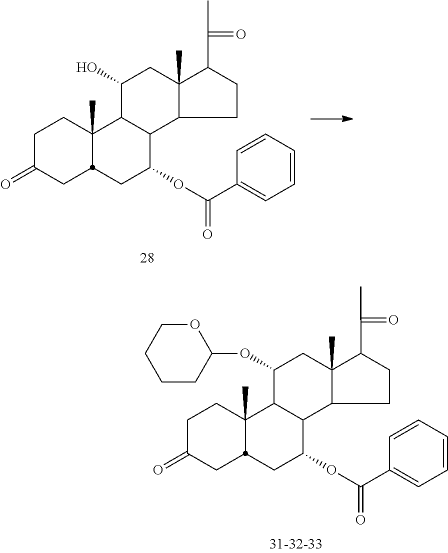

7alpha-Benzoyloxy-11alpha-hydroxy(5beta)pregnan-3,20-dione 28 (807 mg, 1.783 mmol, 1.0 Eq.) was stirred for 3 h at rt° in an extemporaneously prepared solution containing anhydrous THF (20 mL), freshly distilled dihydropyrane (4 mL) and p-toluenesulfonic acid (20 mg). The reaction was quenched with a cold saturated NaHCO₃ solution.

After conventional extraction (ethyl acetate) the crude extract (923 mg) was analyzed by TLC on fluorescent silica gel (petroleum ether/ethyl acetate 2:1) which showed two spots with very close Rf values corresponding to the presence of R/S isomers in the tetrahydropyranyl ether derivative 31 (7α-Benzoyloxy-11α-O-[2′(R+S)]tetrahydropyranyloxy(5β)pregnane-3,20-dione).

The two R/S isomers were separated by flash-chromatography on silica gel (230-400 mesh) using petroleum ether/ethyl acetate 2:1 as eluent. Three fractions were obtained corresponding respectively to the pure less retained isomer 32 (416 mg), the mixture of R/S isomers (340 mg) and the pure more retained isomer 33 (136 mg). The mixture of two R/S isomers of the intermediate fraction (rich in more retained isomer) could be further separated by preparative TLC (dichloromethane/ethyl acetate 2:1, ×2 developments) yielding an additional pure sample of the less retained isomer.

less retained 11alpha-O-[2′(S)]THP isomer 32 (structure confirmed by X-ray crystallographic data)

¹H NMR (CDCl₃) 0.702 (s, 18-CH₃), 1.210 (s, 19-CH₃), 2.141 (s, 21-CH₃), 3.5 & 3.9 (2m, 6′-H, 2H), 4.28 (m, 11-H), 4.63 (m, 2′-H), 5.24 (m, 7-H), 7.47 (m, m-Ar—H), 7.59 (m, p-Ar—H), 7.98 (o-Ar—H).

¹³C NMR (CDCl₃) 38.84 (1), 38.35 (2), 213.56 (3), 45.29 (4), 44.50 (5), 31.21 (6), 71.71 (7), 37.47 (8), 39.95 (9), 36.25 (10), 70.11 (11), 43.17 (12), 43.48 (13), 50.31 (14), 23.79 (15), 23.28 (16), 62.96 (17), 14.08 (18), 22.37 (19), 209.09 (20), 31.50 (21), 129.44 (o-Ar), 128.68 (m-Ar), 133.21 (p-Ar), 130.16 (subst-Ar), 165.52 (Ar—CO), 96.56 (2′-OTHP), 31.96 (3′-OTHP), 21.88 (4′-OTHP), 25.33 (5′-OTHP), 65.76 (6′-OTHP).

more retained 17alpha-O-[2′(R)]THP isomer 33 (structure confirmed by X-ray crystallographic data)

¹H NMR (CDCl₃) 0.645 (s, 18-CH₃), 1.187 (s, 19-CH₃), 2.130 (s, 21-CH₃), 3.5 & 3.9 (2m, 6′-H, 2H), 4.01 (m, 11-H), 4.22 (m, 2′-H), 5.24 (m, 7-H), 7.48 (m, m-Ar—H), 7.61 (m, p-Ar—H), 7.99 (o-Ar—H).

¹³C NMR (CDCl₃) 39.43 (1), 38.33 (2), 212.60 (3), 45.15 (4), 44.27 (5), 31.25 (6), 71.65 (7), 37.86 (8), 39.83 (9), 36.34 (10), 77.22 (11), 47.46 (12), 43.70 (13), 49.93 (14), 23.87 (15), 22.80 (16), 63.16 (17), 13.97 (18), 23.01 (19), 208.77 (20), 31.57 (21), 129.46 (o-Ar), 128.72 (m-Ar), 133.26 (p-Ar), 130.10 (subst-Ar), 165.48 (Ar—CO), 101.79 (2′-OTHP), 31.38 (3′-OTHP), 20.45 (4′-OTHP), 25.26 (5′-OTHP), 63.60 (6′-OTHP).

### Example 6.3

### Preparation of 7β-benzoyloxy-11α-O-[2′(S)]tetrahydropyranyloxy (5β)pregnane-3,20-dione (39) and 7β-benzoyloxy-11α-O-[2′(R)]tetrahydropyranyloxy (5β)pregnane-3,20-dione (40)

Compounds (39) and (40) are prepared as follows:

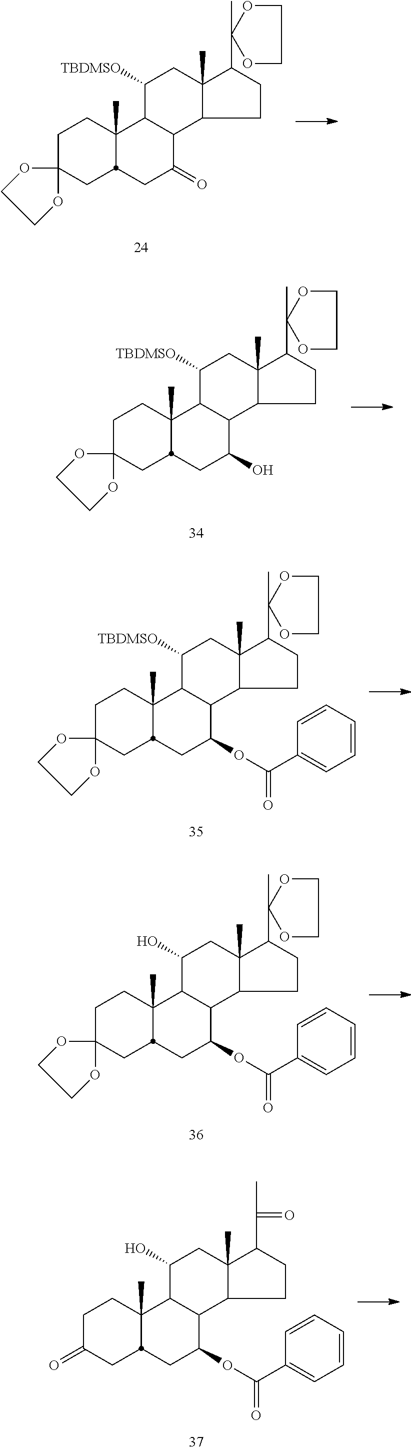

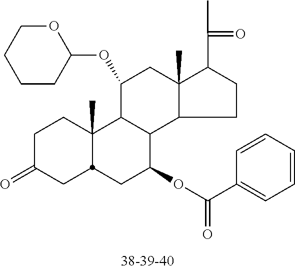

### 3,20-Bis-ethylenedioxy-11α-(tert-Butyl-dimethyl-silanyloxy)(5β)pregnan-7,3-ol (34)

This 7beta-ol compound was obtained as a by-product in the preparation of the 3,20-bisethylenedioxy-11alpha-(tert-butyl-dimethyl-silanyloxy)(5beta)pregnan-7alpha-ol isomer 25 (vide supra). A chromatographic fraction still containing a minor amount of alpha-isomer (Method A) was employed without further purification in the following next step.

### 7β-Benzoyloxy-11α-(tert-Butyl-dimethyl-silanyloxy)(5β)pregnan-3,20-bis-ethyleneketal (35)

The 7beta-hydroxy derivative 34 (400 mg, 0.726 mmol, 1.0 Eq.) was stirred for 12 h at rt° with benzoyl chloride (0.5 mL, 4.307 mmol, 5.93 Eq.) dissolved in pyridine (8 mL). The reaction mixture was cooled at 4° C. and stirred for 30 min after addition of 30 mL of ethyl acetate and 30 mL of a saturated aqueous solution of NaHCO₃.

After conventional extraction (ethyl acetate) and elimination of traces of pyridine by azeotropic evaporation in the presence of n-heptane, the crude extract (491 mg) was analyzed by TLC on fluorescent silica gel (petroleum ether/ethyl acetate 2:1, 3:1 and 4:1).

The product was purified by preparative TLC on fluorescent silica gel (petroleum ether/ethyl acetate 6:1, ×2 developments) to give pure samples of 7beta-benzoate 35 (278 mg) and of 7alpha-benzoate by-product (94 mg).

### 3,20-Bis-ethylenedioxy-7β-benzoyloxy(5β)pregnan-11α-ol (36)

The 7beta-benzoate derivative 35 (250 mg, 0.382 mmol, 1.0 Eq.) dissolved in THF (2.8 mL) was stirred for 72 h at 4° C. after addition of a commercial 1 M solution of tetra-butylammonium fluoride (1.147 mL, 3.01 Eq.) in THF. A same amount of reagent was further added and stirring was prolonged for 24 h at rt°.

After conventional extraction (ethyl acetate) the crude 11alpha-ol 36 (235 mg) was analyzed by TLC on fluorescent silica gel (petroleum ether/ethyl acetate 1:1) and was employed without further purification in the following next step.

### 7β-Benzoyloxy-11α-hydroxy(5β)pregnan-3,20-dione (37)

A solution of bis-ethylenedioxy-11α-ol derivative 36 (235 mg, 0.435 mmol, 1.0 Eq.) in acetone (38 mL) containing p-toluenesulfonic acid monohydrate (64 mg) and small amount of water (1.9 mL) was stirred for 2 days at rt°. The reaction was quenched with a cold saturated NaHCO₃ solution.

After conventional extraction (ethyl acetate) the crude extract (178 mg) was analyzed by TLC on fluorescent silica gel (petroleum ether/ethyl acetate 1:2, 1:3 and ethyl acetate).

The product was purified by preparative TLC on fluorescent silica gel (petroleum ether/ethyl acetate 1:2) to give the 3,20-dioxo-11alpha-ol 37 (43 mg).

7β-Benzoyloxy-11α-O-[2′(R+S)]tetrahydropyranyloxy(5β)pregnane-3,20-dione (38); 7β-Benzoyloxy-11α-O-[2′(S)]tetrahydropyranyloxy(5β)pregnane-3,20-dione (39) and 7β-Benzoyloxy-11α-O-[2′(R)]tetrahydropyranyloxy(5β) pregnane-3,20-dione (40)

The 11alpha-hydroxy derivative 37 (90 mg, 0.199 mmol, 1.0 Eq.) was stirred for 12 h at rt° in an extemporaneously prepared solution containing anhydrous THF (2.25 mL), freshly distilled dihydropyrane (0.45 mL) and p-toluenesulfonic acid (2.25 mg). The reaction was stopped by addition of an excess of a saturated NaHCO₃ solution.

After conventional extraction (ethyl acetate) the residue was analyzed by TLC on fluorescent silica gel (petroleum ether/ethyl acetate 1:1). TLC using petroleum ether/ethyl acetate 2:1 (×2 developments) showed two spots with very close Rf values corresponding to the presence of R/S isomers in the tetrahydropyranylether derivative 38.

These two isomers were separated by preparative TLC in similar conditions to give pure samples of the less retained isomer 39 (43 mg) and of the more retained isomer 40 (31 mg).

less retained 11alpha-O-[2′(S)]THP isomer 39

¹H NMR (CDCl₃) 0.707 (s, 18-CH₃), 1.209 (s, 19-CH₃), 2.122 (s, 21-CH₃), 3.5 & 3.9 (2m, 6′-H, 2H), 4.21 (m, 11-H), 4.56 (m, 2′-H), 5.19 (m, 7-H), 7.47 (m, m-Ar—H), 7.59 (m, p-Ar—H), 8.03 (o-Ar—H).

¹³C NMR (CDCl₃) 38.96 (1), 38.52 (2), 211.84 (3), 44.48 (4), 50.50 (5), 33.73 (6), 71.50 (7), 37.58 (8), 41.01 (9), 37.30 (10), 70.94 (11), 43.52 (12), 43.27 (13), 51.16 (14), 23.82 (15), 23.25 (16), 62.92 (17), 14.16 (18), 10.21 (19), 209.06 (20), 31.40 (21), 129.63 (o-Ar), 128.53 (m-Ar), 133.16 (p-Ar), 130.33 (subst-Ar), 165.61 (Ar—CO), 97.44 (2′-OTHP), 32.15 (3′-OTHP), 22.06 (4′-OTHP), 25.31 (5′-OTHP), 65.90 (6′-OTHP).

more retained 11alpha-O-[2′(R)]THP isomer 40

¹H NMR (CDCl₃) 0.660 (s, 18-CH₃), 1.172 (s, 19-CH₃), 2.115 (s, 21-CH₃), 3.5 & 3.9 (2m, 6′-H, 2H), 3.91 (m, 11-H), 4.63 (m, 2′-H), 5.19 (m, 7-H), 7.49 (m, m-Ar—H), 7.61 (m, p-Ar—H), 8.04 (o-Ar—H).

¹³C NMR (CDCl₃) 39.61 (1), 38.30 (2), 210.89 (3), 44.37 (4), 50.12 (5), 33.80 (6), 70.86 (7), 37.96 (8), 40.91 (9), 37.15 (10), 78.36 (11), 47.33 (12), 43.57 (13), 51.19 (14), 23.92 (15), 22.75 (16), 63.13 (17), 14.09° (18), 11.49 (19), 208.78 (20), 31.51 (21), 129.63 (o-Ar), 128.56 (m-Ar), 133.19 (p-Ar), 130.29 (subst-Ar), 165.60 (Ar—CO), 101.81 (2′-OTHP), 31.62 (3′-OTHP), 20.61 (4′-OTHP), 25.25 (5′-OTHP), 63.74 (6′-OTHP).

### Example 6.4

### Preparation of 7β,11α-dibenzoyloxy(5β)pregnane-3,20-dione (41)

Compound (41) is prepared according to the following reaction scheme:

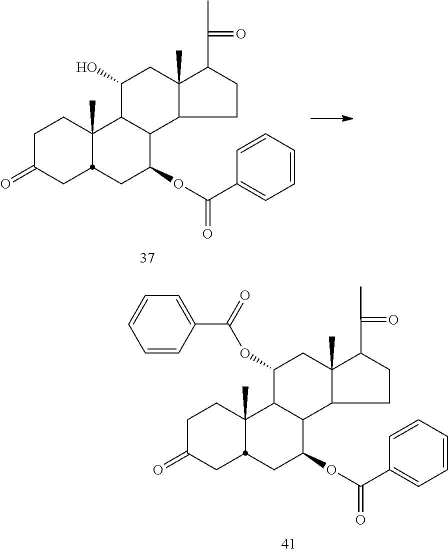

7β,11α-Dibenzoyloxy(5β)pregnane-3,20-dione (41)

The 11alpha-hydroxy derivative 37 (45 mg, 0.099 mmol, 1.0 Eq.) was stirred for 12 h at rt° with benzoyl chloride (0.068 mL, 0.586 mmol, 5.89 Eq.) dissolved in pyridine (1.1 mL). The reaction mixture was cooled in an ice-bath at 4° C. and stirred for 30 min after addition of 4 mL of ethyl acetate and 4 mL of a saturated aqueous solution of NaHCO₃.

After conventional extraction (ethyl acetate) and elimination of traces of pyridine by azeotropic evaporation in the presence of n-heptane, the crude extract (60 mg) was analyzed by TLC on fluorescent silica gel (petroleum ether/ethyl acetate 2:1).

The crude product was purified by preparative TLC on fluorescent silica gel (petroleum ether/ethyl acetate 2:1) to give a pure sample of dibenzoate 41 (26 mg).

¹H NMR (CDCl₃) 0.800 (s, 18-CH₃), 1.226 (s, 19-CH₃), 2.075 (s, 21-CH₃), 5.28 (m, 7-H), 5.57 (m, 11-H), 7.51 (m, m-Ar—H, 4H), 7.61 (m, p-Ar—H, 2H), 8.07 (m, o-Ar—H, 4H).

¹³C NMR (CDCl₃) 39.46 (1), 38.22 (2), 210.46 (3), 44.94 (4), 50.00 (5), 33.82 (6), 70.55 (7), 37.90 (8), 40.82 (9), 37.21 (10), 71.54 (11), 44.21 (12), 43.62 (13), 50.25 (14), 23.83 (15), 22.86 (16), 62.88 (17), 13.94 (18), 11.12 (19), 208.27 (20), 31.46 (21), 129.60/65 (o-Ar/7+11), 128.60/63 (m-Ar/7+11), 133.32 (p-Ar/7+11), 130.26/28 (subst-Ar/7+11), 165.53 (Ar—CO/7+11).

### Example 7

### Preparation of 11,17-disubstituted (5β-H)pregnane-3,20-dione derivatives (51), (52), (53) and (54)

The compounds (51), (52), (53) and (54) are prepared according to the following reaction scheme:

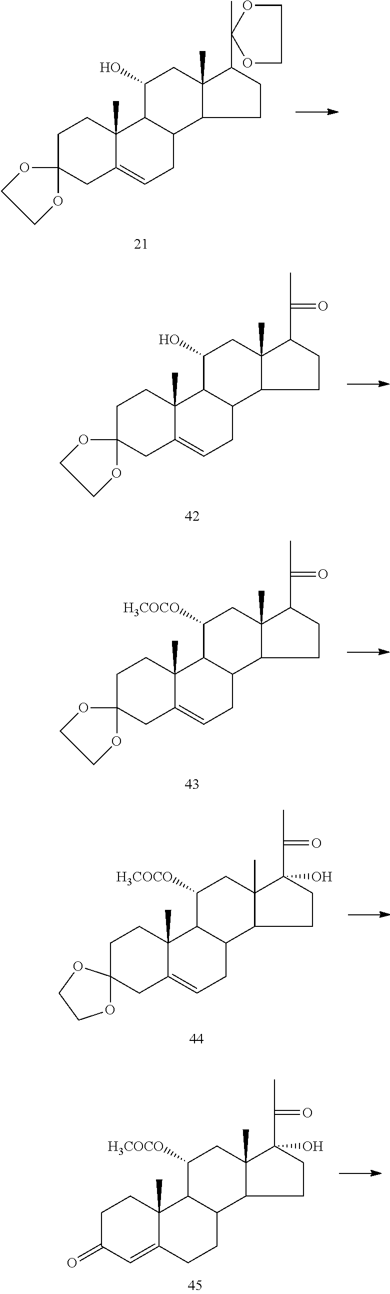

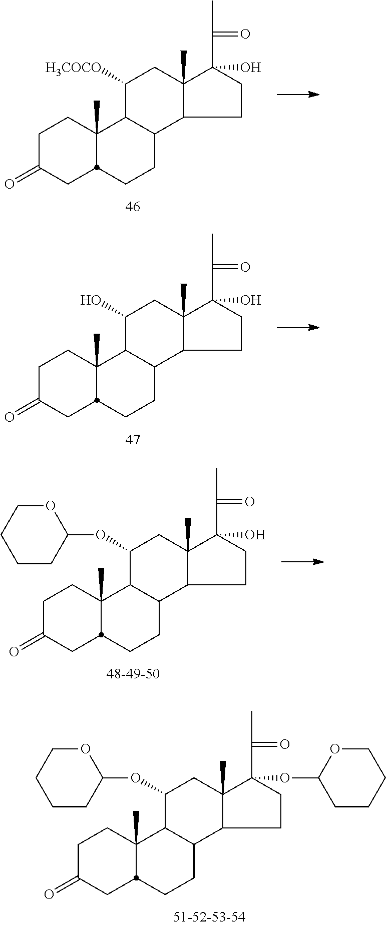

### 3-ethylenedioxy-11α-hydroxypregn-5-en-20-one (42)

A solution of 3,20-bis-ethylenedioxypregn-5-en-11α-ol 21 (10.0 g, 23.891 mmol, 1.0 Eq.) in acetone (1000 mL) containing p-toluenesulfonic acid monohydrate (300 mg) dissolved in acetone (30 mL) and a small amount of water (3.75 mL) was magnetically stirred under nitrogen at rt°. The reaction was monitored by TLC at regular intervals and stopped when selective hydrolysis of the 20-ketal exhibited an optimal yield. The reaction mixture was quenched with a cold saturated NaHCO₃ solution.

After conventional extraction (dichloromethane) the crude product was recrystallized from a dichloromethane-methanol mixture containing 1% pyridine to give the pure 20-ketone.

The dry product containing the 3-monoketal 42 (6.0 g) was analyzed by TLC on fluorescent silica gel (petroleum ether/ethyl acetate 1:2).

### 3-Ethylenedioxy-11α-acetoxypregn-5-en-20-one (43)

The 11alpha-hydroxy derivative 42 (15 g, 40.052 mmol, 1.0 Eq.) was stirred for 12 h at rt° with an excess of acetic anhydride (60 mL) dissolved in pyridine (300 mL).

The reaction mixture was evaporated under reduced pressure. Traces of acetic anhydride were eliminated by evaporation of ethanol whereas residual pyridine was eliminated by azeotropic evaporation in the presence of n-heptane.

The dry product (16.5 g) was analyzed by TLC on fluorescent silica gel (petroleum ether/ethyl acetate 1:1; dichloromethane/ethyl acetate 1:1).

The crude product was purified by flash-chromatography on silica gel (230-400 mesh) using dichloromethane/ethyl acetate 1:1 as eluent to give the pure 11-acetate 43.

### 3-Ethylenedioxy-11α-acetoxy-17α-hydroxypregn-5-en-20-one (44)

A suspension of NaH (3.4 g of dry solid obtained after eliminating mineral oil from the 80% commercial product with anhydrous n-hexane) in a mixture of freshly distilled anhydrous t-butanol (26 mL) and DMF (32 mL) was sonicated for 45 min under nitrogen atmosphere, then cooled at −25° C. (acetone+dry ice bath) and magnetically stirred. To this magnetically stirred suspension was added triethylphosphite (2.9 mL) then the 3-ethylenedioxy-11α-acetoxypregn-5-en-20-one derivative 43 (4.9 g, 11.763 mmol, 1.0 Eq.) dissolved in a mixture of freshly distilled anhydrous THF (54 mL) and DMF (11 mL). A stream of oxygen was bubbled rather strongly into the reaction mixture for 2 h at −25° C. After the oxygen flow was interrupted, the reaction mixture was neutralized with acetic acid and diluted with dichloromethane (500 mL) and water (300 mL).

The aqueous layer was separated and extracted several times with dichloromethane. The combined organic layers were washed with water, filtered on a phase-separating paper (Whatman) and evaporated to dryness under reduced pressure.

The crude extract was analyzed by TLC on fluorescent silica gel (petroleum ether/ethyl acetate 1:3 Rf 0.31).

The product was purified by flash-chromatography on silica gel (230-400 mesh) using petroleum ether/ethyl acetate 1:3 as eluent to give the pure 17alpha-hydroxy derivative 44 (2.0 g).

### 11α-Acetoxy-17α-hydroxypregn-4-ene-3,20-dione (45)

A solution of 3-ethylenedioxy-derivative 44 (400 mg, 0.925 mmol, 1.0 Eq.) in acetone (48 mL) containing p-toluenesulfonic acid monohydrate (80 mg) dissolved in acetone (48 mL) and a small amount of water (2.4 mL) was magnetically stirred under nitrogen for 2 days at rt°. The reaction was quenched with a cold saturated NaHCO₃ solution.

After conventional extraction (ethyl acetate) the residue containing the 3-oxo product 45 (357 mg) was analyzed by TLC on fluorescent silica gel (petroleum ether/ethyl acetate 1:1 and 2:1) and was employed without further purification in the following next step.

### 11α-Acetoxy-17α-hydroxy(5β)pregnane-3,20-dione (46)

11α-Acetoxy-17α-hydroxypregn-4-ene-3,20-dione 45 (356 mg, 0.916 mmol, 1.0 Eq.) dissolved in a dioxane-95% ethanol 1:1 v/v mixture containing 0.8% pyridine (42 mL) was introduced in a glass hydrogenation apparatus and magnetically stirred under hydrogen at atmospheric pressure, for 12 h at 30° C., in the presence of 10% Pd—C catalyst (350 mg).

The reaction mixture was filtered on a pad of ™Celite and evaporated under reduced pressure. The residual pyridine was eliminated by azeotropic distillation in the presence of n-heptane.

The dry product (357 mg) was analyzed by TLC on fluorescent silica gel (petroleum ether/ethyl acetate 1:1) to give a major non-UV-absorbing spot. This 4,5-dihydrogenated product 46 was employed without further purification in the following next step.

### 11α,17α-Dihydroxy(5β)pregnane-3,20-dione (47)

The 11α-acetoxy derivative 46 (356 mg, 0.912 mmol, 1.0 Eq.) was stirred in a solution of K₂CO₃ (400 mg) in a mixture of methanol (35 mL) and water (5 mL) for 48 h at 50° C. under nitrogen atmosphere.

The reaction mixture was cooled in an ice-bath, neutralized with a 1 M aqueous solution of HCl.

After conventional extraction (ethyl acetate) the dry 17-hydroxy product 47 (308 mg) was analyzed by TLC on fluorescent silica gel (petroleum ether/ethyl acetate 1:1 and 1:2) and was employed without further purification in the following next step.

### 11α-O-[2′(R+S)]Tetrahydropyranyloxy-17α-hydroxy(5β)pregnane-3,20-dione (48); 11α-O-[2′(S)]Tetrahydropyranyloxy-17α-hydroxy(5β)pregnane-3,20-dione (49) and 11α-O-[2′(R)]Tetrahydropyranyloxy-17α-hydroxy(5β)pregnane-3,20-dione (50)

The 11α,17α-diol 47 (100 mg, 0.287 mmol, 1.0 Eq.) was stirred for 4 h at rt° in an extemporaneously prepared solution containing anhydrous THF (0.833 mL), freshly distilled dihydropyrane (0.166 mL) and p-toluenesulfonic acid (1 mg). The reaction was stopped by addition of an excess of a saturated NaHCO₃ solution.

After conventional extraction (dichloromethane) the residue was analyzed by TLC on fluorescent silica gel (petroleum ether/ethyl acetate 1:1). TLC using petroleum ether/ethyl acetate 2:1 (×3 developments) revealed two spots with very close Rf values corresponding to the presence of R/S isomers in the tetrahydropyranylether derivative 48. These two isomers were separated by preparative TLC in similar conditions to give partially purified samples respectively enriched in the less retained isomer 49 (47 mg) and in the more retained isomer 50 (49 mg).

### 11α,17α-Bis-O-[2′(S),2′(R)]tetrahydropyranyloxy(5β)pregnane-3,20-dione (51); 11α,17α-Bis-O-[2′(R),2′(R)]tetrahydropyranyloxy(5β)pregnane-3,20-dione (52); 11α,17α-Bis-O-[2′(S),2′(S)]Tetrahydropyranyloxy(5β)pregnane-3,20-dione (53) and 11α,17α-Bis-O-[2′(R),2′(S)]Tetrahydropyranyloxy(5β)pregnane-3,20-dione (54)

The partially purified samples of 11α-O-[2′(R)] and [2′(S)]tetrahydropyranyloxy-17α-hydroxy(5beta)pregnane-3,20-dione R/S isomers 49 and 50 (36 mg, 0.083 mmol, 1.0 Eq.) were stirred for 4 h at rt° in an extemporaneously prepared solution containing anhydrous THF (0.416 mL), freshly distilled dihydropyrane (0.083 mL) and p-toluenesulfonic acid (1 mg). The reaction was stopped by addition of an excess of a saturated NaHCO₃ solution.

After conventional extraction (ethyl acetate) the residue was analyzed by TLC on fluorescent silica gel (petroleum ether/ethyl acetate, 4:1). TLC using petroleum ether/ethyl acetate 4:1 revealed two major spots with very close Rf values corresponding to the presence of 17alpha-O-tetrahydropyranylether R/S isomers.

These two isomers were separated by preparative TLC in similar conditions.

The extract corresponding to the less retained starting product 49 (36 mg) gave two fractions corresponding to a less retained spot (8.8 mg) containing the pure isomer 53 and to a more retained spot (13.2 mg) containing isomer 51 as the major product still contaminated with other isomers as minor by-products.

The extract corresponding to the more retained starting product 50 (28 mg) gave two fractions corresponding to a less retained spot (9.1 mg) containing the above-mentioned isomer 51, as major compound and to a more retained spot (15.8 mg) containing isomer 52 as the major product. These two fractions still contained other isomers as minor by-products (the presence of traces of isomer 54 could not be unambiguously characterized in ¹³C NMR spectra).

isomer 11alpha,17alpha-bis-O-[2′(S),2′(R)]tetrahydropyranyloxy 51

¹H NMR (CDCl₃) 0.588 (s, 18-CH₃), 1.137 (s, 19-CH₃), 2.154 (s, 21-CH₃), 3.5 & 3.9 (2m, 6′-H, 4H), 4.12 (m, 11-H), 4.37-4.65 (m, 2′-H, 2H).

¹³C NMR (CDCl₃) 39.34 (1), 38.52 (2), 214.72 (3), 42.85 (4), 46.38 (5), 25.98 (6), 26.82 (7), 34.71 (8), 44.78 (9), 36.05 (10), 70.86 (11), 36.13 (12), 47.54 (13), 50.56 (14), 23.89 (15), 26.55 (16), 97.40 (17), 15.79 (18), 22.72 (19), 209.54 (20), 27.43 (21), 96.11 (2′-OTHP/11), 31.81 (3′-OTHP/11), 21.56 (4′-OTHP/11), 25.37 (5′-OTHP/11), 65.24 (6′-OTHP/11), 97.08 (2′-OTHP/17), 31.91 (3′-OTHP/17), 20.56 (4′-OTHP/17), 25.10 (5′-OTHP/17), 63.78 (6′-OTHP/17).

isomer 11alpha,17alpha-bis-O-[2′(R),2′(R)]tetrahydropyranyloxy 52

¹H NMR (CDCl₃) 0.577 (s, 18-CH₃), 1.111 (s, 19-CH₃), 2.124 (s, 21-CH₃), 3.5 & 3.9 (2m, 6′-H, 4H), 3.82 (m, 11-H), 4.35-4.66 (m, 2′-H, 2H).

¹³C NMR (CDCl₃) 39.58 (1), 38.35 (2), 213.90 (3), 42.74 (4), 46.10 (5), 26.10 (6), 26.83 (7), 35.00 (8), 45.05 (9), 36.02 (10), 79.22 (11), 40.14 (12), 47.59 (13), 50.05 (14), 23.89 (15), 26.34 (16), 96.27 (17), 15.63 (18), 23.46 (19), 209.93 (20), 27.08 (21), 101.28 (2′-OTHP/11), 31.26 (3′-OTHP/11), 19.82 (4′-OTHP/11), 25.40 (5′-OTHP/11), 62.61 (6′-OTHP/11), 97.13 (2′-OTHP/17), 31.94 (3′-OTHP/17), 20.58 (4′-OTHP/17), 25.11 (5′-OTHP/17), 63.84 (6′-OTHP/17).

isomer 11alpha,17alpha-bis-O-[2′(S),2′(S)]tetrahydropyranyloxy 53

¹H NMR (CDCl₃) 0.595 (s, 18-CH₃), 1.135 (s, 19-CH₃), 2.216 (s, 21-CH₃), 3.5 & 3.9 (2m, 6′-H, 4H), 4.11 (m, 11-H), 4.46-4.57 (m, 2′-H, 2H).

¹³C NMR (CDCl₃) 39.28 (1), 38.49 (2), 214.72 (3), 42.84 (4), 46.34 (5), 26.01 (6), 26.80 (7), 34.59 (8), 44.87 (9), 36.04 (10), 70.95 (11), 36.04 (12), 47.10 (13), 50.23 (14), 23.71 (15), 23.63 (16), 94.50 (17), 15.77 (18), 22.73 (19), 209.70 (20), 27.67 (21), 96.28 (2′-OTHP/11), 31.85 (3′-OTHP/11), 21.66 (4′-OTHP/11), 25.37 (5′-OTHP/11), 65.34 (6′-OTHP/11), 96.53 (2′-OTHP/17), 31.85 (3′-OTHP/17), 21.66 (4′-OTHP/17), 25.18 (5′-OTHP/17), 65.46 (6′-OTHP/17).

### Example 8

### Preparation of 7,12-disubstituted (5β-H)cholane derivatives (57), (59), (60), (63), (64), (71), (73) and (74)

### Example 8.1

### Preparation of methyl 3-oxo-7α,12α-dibenzoyloxycholanate (57)

Compound (57) is prepared according to the following reaction scheme:

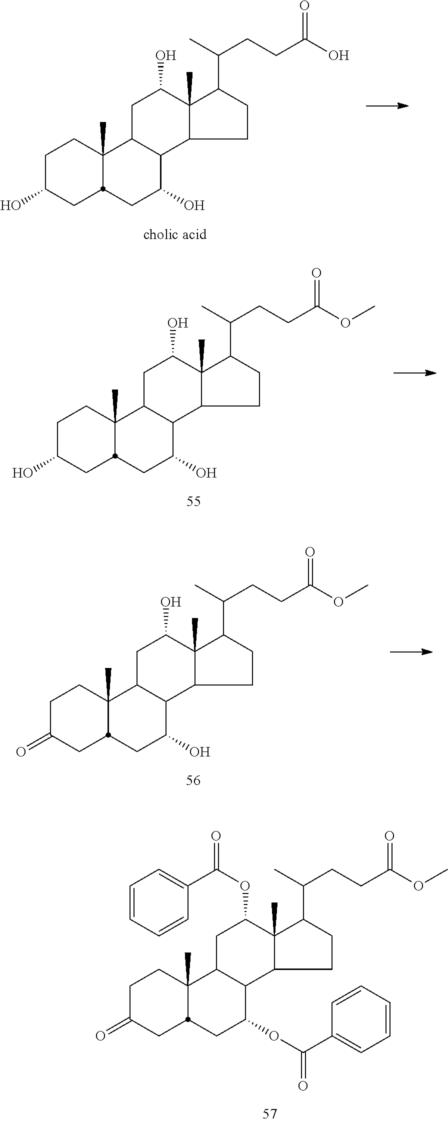

### Methyl 3α,7α,12α-trihydroxycholanate (“methyl cholate”) (55)

3alpha,7alpha,12alpha-Trihydroxy(5beta-H)cholan-24-oic acid (“cholic acid”) (from Aldrich) (10.0 g, 24.5 mmol, 1.0 Eq.) dissolved in methanol (200 mL) was stirred for 3 h in the presence of 2.5 mL of conc. HCl. The reaction was stopped by addition of an excess of a saturated solution of NaHCO₃.

After conventional extraction (ethyl acetate) the white extract was analyzed by TLC on fluorescent silica gel (chloroform/methanol 10:1), showing a single spot well above the starting product corresponding to the methyl ester 55.

### Methyl 3-oxo-7alpha,12alpha-dihydroxycholanate (56)

Methyl 3alpha,7alpha,12alpha-trihydroxycholanate 55 (200 mg, 0.473 mmol, 1.0 Eq.) dissolved in toluene (40 mL) was magnetically stirred for 12 h under reflux in a Dean-Stark water trap in the presence of 2.0 g of Ag₂CO₃/Celite reagent (cf Fieser L., Reagents for Organic Synthesis, Vol 2, p. 363; Fetizon M., Balogh V., Golfier M., J. Org. Chem. (1971), 36, 1339-41).

The reaction mixture was filtered on a column of Celite which was washed with an excess of toluene. The combined filtrates were evaporated under reduced pressure.

The white extract was analyzed by TLC on fluorescent silica gel (chloroform/ethyl acetate 1:3), showing a major spot above the starting product. This mono-3-oxo product 56 was employed without further purification in the following next step.

### Methyl 3-oxo-7alpha,12alpha-dibenzoyloxycholanate (57)

The 7alpha,12alpha-diol 56 (100 mg, 0.238 mmol, 1.0 Eq.) was stirred for 12 h at rt° with an excess of benzoyl chloride (0.20 mL, 1.721 mmol, 7.2 Eq.) dissolved in a mixture of pyridine (0.3 mL) and dichloromethane (0.3 mL). The reaction mixture was cooled in an ice-bath at 4° C. and stirred for 30 min after addition of 3 mL of ethyl acetate and 3 mL of a saturated aqueous solution of NaHCO₃.

After conventional extraction (ethyl acetate) and elimination of traces of pyridine by azeotropic evaporation in the presence of n-heptane, the crude extract was analyzed by TLC on fluorescent silica gel (petroleum ether/ethyl acetate 3:1) which showed a minor amount of monobenzoylated by-product.

The crude product was purified by preparative TLC in similar conditions to give a pure sample of dibenzoate 57.

¹H NMR (CDCl₃) 0.825 (d, 21-CH₃), 0.876 (s, 18-CH₃), 1.102 (s, 19-CH₃), 3.579 (s, OCH₃), 5.32 (m, 7-H), 5.44 (m, 12-H), 7.47 (m, m-Ar—H, 4H), 7.64 (m, p-Ar—H, 2H), 8.04 (o-Ar—H, 4H).

¹³C NMR (CDCl₃) 36.67 (1), 36.37 (2), 212.11 (3), 44.69 (4), 42.10 (5), 31.16 (6), 71.40 (7), 38.53 (8), 30.36 (9), 34.66 (10), 25.88 (11), 75.90 (12), 45.69 (13), 43.74 (14), 23.12 (15), 27.27 (16), 48.05 (17), 12.39 (18), 21.74 (19), 34.75 (20), 17.65 (21), 30.99 (22), 30.82 (23), 174.53 (24), 51.57 (OCH₃), 129.48/51 (o-Ar/7+12), 128.89/92 (m-Ar/7+12), 133.33/41 (p-Ar/7+12), 130.52/58 (subst-Ar/7+12), 165.49/70 (Ar—CO/7+12).

### Example 8.2

### Preparation of methyl 3-oxo-7α-benzoyloxy-12α-O-[2′(S)]tetrahydropyranyloxycholanate (59) and methyl 3-oxo-7α-benzoyloxy-12α-O-[2′(R)]tetrahydropyranyloxy-cholanate (60)

Compounds (59) and (60) are prepared according to the following reaction scheme:

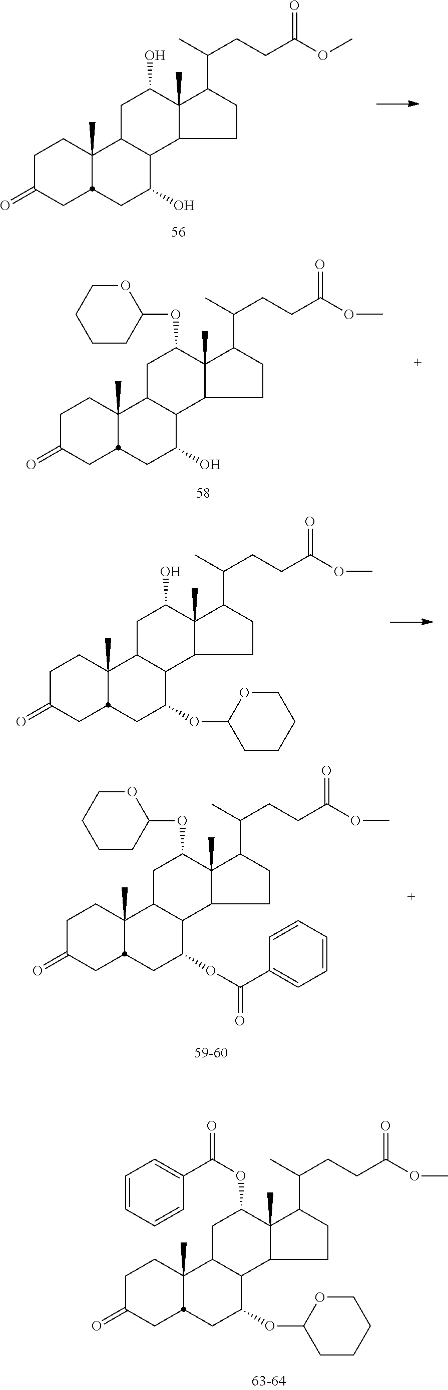

### Methyl 3-oxo-7α-hydroxy-12α-tetrahydropyranyloxycholanate (58)

The 7alpha,12alpha-diol 56 (250 mg, 0.594 mmol, 1.0 Eq.) was stirred for 12 h at rt° with a limited amount of an extemporaneously prepared solution containing anhydrous THF (0.72 mL), freshly distilled dihydropyrane (0.06 mL 0.658 mmol, 1.1 Eq.) and p-toluenesulfonic acid (0.3 mg). The incomplete reaction was stopped by addition of an excess of a saturated NaHCO₃ solution.

After conventional extraction (dichloromethane), the crude extract was analyzed by TLC on fluorescent silica gel (petroleum ether/ethyl acetate 1:1, petroleum ether/MTBE 1:1, chloroform/ethyl acetate 1:1).

The mixture of products containing the 12-mono-tetrahydropyranylether 58 was purified by TLC on fluorescent silica gel (petroleum ether/MTBE 1:1, ×4 developments) to give six different fractions controlled by analytical TLC in similar conditions: i) a less retained fraction (42 mg) at a much higher Rf corresponding probably to the bis-tetrahydropyranyl ether, ii) four major well-separated intermediate fractions of decreasing Rf values (A: 54 mg, B: 65 mg, C: 29 mg and D: 34 mg) and iii) a highly retained fraction (36 mg) corresponding to the unreacted diol 56. The four intermediate fractions were employed without further purification in the next benzoylation step.

### Methyl 3-oxo-7α-benzoyloxy-12α-O-[2′(S)]tetrahydropyranyloxycholanate (59) and methyl 3-oxo-7α-benzoyloxy-12α-O-[2′(R)]tetrahydropyranyloxy-cholanate (60)

The preceding monohydroxylated chromatographic fractions A, B, C and D (54 mg, 0.106 mmol, 65 mg, 0.129 mmol, 29 mg, 0.058 mmol and 34 mg, 0.068 mmol, respectively), containing the mixture of R/S isomers of mono-12alpha-tetrahydropyranyl ether 58 contaminated with isomeric 7-tetrahydropyranyloxy by-products were stirred for 48 h at rt° with an excess of benzoyl chloride (0.050 mL, 0.431 mmol, 0.045 mL, 0.388 mmol, 0.100 mL, 0.861 mmol and 0.080 mL, 0.689 mmol, respectively) dissolved in pyridine (0.80 mL, 0.70 mL, 1.50 mL and 1.30 mL, respectively). The reaction was complete after 48 h for the two fractions C and D but was much slower for fractions A and B which were not totally benzoylated after 6 days at rt°. After this reaction time, the reaction mixture was cooled in an ice-bath at 4° C. and stirred for 30 min after addition of 1.5 mL of ethyl acetate and 1.5 mL of a saturated aqueous solution of NaHCO₃.

After conventional extraction (ethyl acetate) and elimination of traces of pyridine by azeotropic evaporation in the presence of n-heptane, the crude benzoylated products, easily detected by UV absorption, were purified by preparative TLC (petroleum ether/MTBE 2:1, ×2 or 3 developments):

TLC of benzoylated fraction A gave a pure sample (22 mg) of 7alpha-monobenzoate-12alpha-O-[2′(S)]tetrahydropyranylether 59; TLC of benzoylated fraction B gave a pure sample (21 mg) of 7alpha-monobenzoate-12alpha-O-[2′(R)]tetrahydropyranylether-12alpha-monobenzoate 60; TLC of benzoylated fraction C gave a pure sample (22 mg) of 7alpha-O-[2′(S)]tetrahydropyranylether-12alpha-monobenzoate 63; and TLC of benzoylated fraction D gave a pure sample (28 mg) of 7alpha-O-[2′(R)]tetrahydropyranylether-12alpha-monobenzoate 64.

The purified products were analyzed by TLC on fluorescent silica gel (petroleum ether/MTBE 1:1; petroleum ether/MTBE 2:1, ×4 developments). The order of Rf values of benzoylated derivatives (Rf of 60=Rf of 64>Rf of 63>Rf of 59) was found to differ from that mentioned above for hydroxylated precursors and was much less prone to an efficient separation of the two 7- and 12-benzoylated families.

A direct synthesis of compounds 63 and 64 and their NMR characterization is mentioned hereafter.

more retained 12alpha-O-[2′(S)]THP isomer 59 (more retained in petroleum ether/ethyl acetate but less retained in chloroform/ethyl acetate)

¹H NMR (CDCl3) 0.769 (s, 18-CH3), 1.004 (d, 21-CH3), 1.087 (s, 19-CH3), 3.63 (s, OCH3), 3.5 & 3.9 (2m, 6′-H, 2H), 4.05 (m, 12-H), 4.84 (m, 2′-H). 5.20 (m, 7-H), 7.42 (m, m-Ar—H, 2H), 7.56 (m, p-Ar—H, 1H), 7.97 (o-Ar—H, 2H).

¹³C NMR (CDCl3) 36.97 (1), 36.69 (2), 212.45 (3), 44.86 (4), 42.10 (5), 31.30 (6), 71.76 (7), 38.79 (8), 29.24 (9), 34.85 (10), 23.96 (11), 77.07 (12), 46.58 (13), 42.42° (14), 23.58 (15), 28.05 (16), 45.98 (17), 12.68 (18), 21.85 (19), 36.15 (20), 17.25 (21), 31.22 (22), 31.22 (23), 174.85 (24), 51.59 (OCH₃), 129.55 (o-Ar), 128.75 (m-Ar), 133.18 (p-Ar), 130.63 (subst-Ar), 165.59 (Ar—CO), 95.50 (2′-OTHP), 32.49 (3′-OTHP), 19.65 (4′-OTHP), 25.78 (5′-OTHP), 62.69 (6′-OTHP).

less retained 12alpha-O-[2′(R)]THP isomer 60 (less retained in petroleum ether/ethyl acetate but more retained in chloroform/ethyl acetate)(tentative assignments from unseparated mixture with the 7alpha-O-[2′(S)]THP isomer 63)

¹H NMR (CDCl₃) 0.725 (s, 18-CH₃), 0.897 (d, 21-CH₃), 1.068 (s, 19-CH₃), 3.63 (s, OCH₃), 3.5 & 3.9 (2m, 6′-H, 2H), 3.86 (m, 12-H), 4.70 (m, 2′-H). 5.22 (m, 7-H), 7.42 (m, m-Ar—H, 2H), 7.57 (m, p-Ar—H, 1H), 8.02 (m, o-Ar—H, 2H).

¹³C NMR (CDCl₃) 36.72 (1), 36.72 (2), 213.24 (3), 44.95 (4), 42.52 (5), 31.34 (6), 71.92 (7), 38.79 (8), 29.43 (9), 35.07 (10), 26.57 (11), 82.10 (12), 46.72 (13), 42.61 (14), 23.39 (15), 27.54 (16), 46.44 (17), 12.29 (18), 21.84 (19), 35.31 (20), 17.99 (21), 31.07 (22), 30.96 (23), 174.72 (24), 51.60 (OCH₃), 129.61 (o-Ar), 128.66 (m-Ar), 133.14 (p-Ar), 130.74 (subst-Ar), 165.79 (Ar—CO), 100.33 (2′-OTHP), 31.72 (3′-OTHP), 19.84 (4′-OTHP), 25.95 (5′-OTHP), 62.89 (6′-OTHP).

### Example 8.3

### Preparation of methyl 3-oxo-12α-benzoyloxy-7α-O-[2′(S)]tetrahydropyranyloxycholanate (63) and methyl 3-oxo-12α-benzoyloxy-7α-O-[2′(R)]tetrahydropyranyloxycholanate (64)

Compounds (63) and (64) are prepared according to the following reaction scheme:

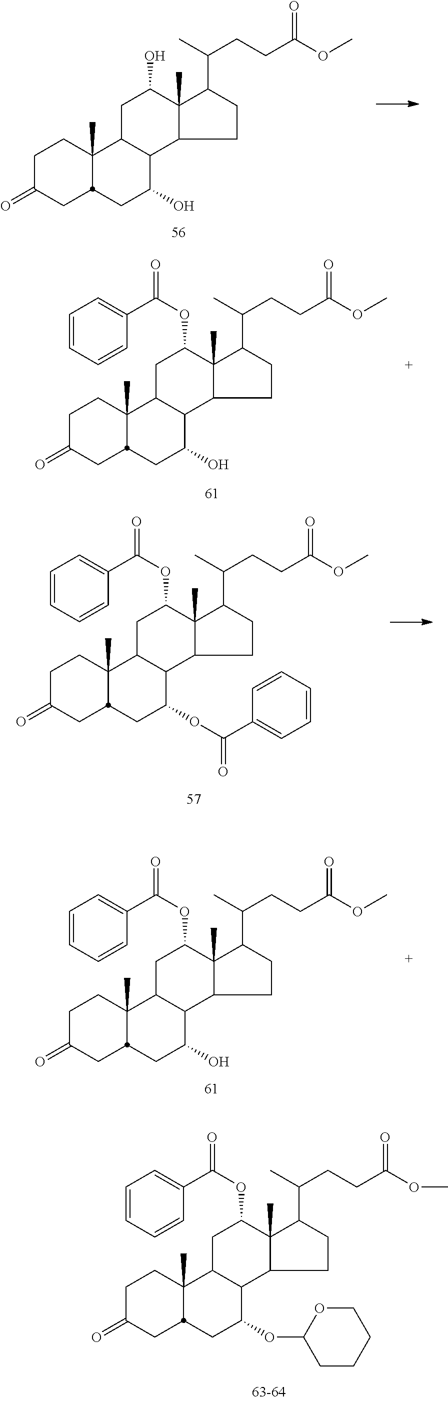

### Methyl 3-oxo-12α-benzoyloxy-7α-hydroxycholanate (61) and methyl 3-oxo-7α,12α-dibenzoyloxycholanate (57)

The 7alpha,12alpha-diol 56 (343 mg, 0.82 mmol, 1.0 Eq.) was stirred for 24 h at rt° with a limited amount of benzoyl chloride (0.121 mL, 1.04 mmol, 1.28 Eq.) dissolved in a mixture of pyridine (1.3 mL) and dichloromethane (1.3 mL). The reaction mixture was cooled in an ice-bath at 4° C. and stirred for 30 min after addition of 10 mL of ethyl acetate and 10 mL of a saturated aqueous solution of NaHCO₃.

After conventional extraction (ethyl acetate) and elimination of traces of pyridine by azeotropic evaporation in the presence of n-heptane, the dry extract was analyzed by TLC on fluorescent silica gel (cyclohexane/ethyl acetate 7:3) which showed a minor amount of dibenzoylated by-product.

The crude product was purified by column chromatography on silica gel in similar conditions to give the pure mono-12alpha-benzoate 61 from the less retained fraction (228 mg) and the 7alpha,12alpha-dibenzoate by-product 57 from the more retained fraction (179 mg).

### Methyl 3-oxo-12α-benzoyloxy-7α-O-[2′(R+S)]tetrahydropyranyloxy-cholanate (62); methyl 3-oxo-12α-benzoyloxy-7α-O-[2′(S)]tetrahydro-pyranyloxycholanate (63) and methyl 3-oxo-12α-benzoyloxy-7α-O-[2′(R)]tetra-hydropyranyloxycholanate (64)

The 7alpha-hydroxy derivative 61 (100 mg, 0.19 mmol, 1.0 Eq.) was stirred for 4 days at rt° in an extemporaneously prepared solution containing anhydrous THF (2 mL), freshly distilled dihydropyrane (0.775 mL) and p-toluenesulfonic acid (6 mg). The reaction was stopped by addition of an excess of a saturated NaHCO₃ solution.

After conventional extraction (ethyl acetate) the residue was analyzed by TLC on fluorescent silica gel (petroleum ether/ethyl acetate, 2:1, cyclohexane/ethyl acetate 9:1). TLC using cyclohexane/ethyl acetate 9:1 showed two spots with very close Rf values corresponding to the presence of R/S isomers in the crude tetrahydropyranylether derivative 62. This product was then purified by column chromatography (cyclohexane/ethyl acetate 7:3 to give a mixture (141 mg) of R/S isomers. A part (40 mg) of this mixture of both isomers was separated by preparative TLC (cyclohexane/ethyl acetate 9:1, ×2 developments then cyclohexane/ethyl acetate 7:3, ×3 developments) to provide pure samples of the more retained isomer 63 (5 mg) and of the less retained isomer 64 (8 mg).

more retained 7alpha-O-[2′(S)]THP isomer 63

¹H NMR (CDCl₃) 0.837 (s, 18-CH₃), 0.867 (d, 21-CH₃), 1.034 (s, 19-CH₃), 3.61 (s, OCH₃), 3.5 & 3.9 (2m, 6′-H, 2H), 3.88 (m, 7-H), 4.71 (m, 2′-H). 5.43 (m, 12-H), 7.45 (m, m-Ar—H, 2H), 7.58 (m, p-Ar—H, 1H), 8.07 (o-Ar—H, 2H).

¹³C NMR (CDCl₃) 36.67 (1), 36.61 (2), 213.09 (3), 45.48 (4), 42.78 (5), 29.42 (6), 72.18 (7), 39.57 (8), 28.80 (9), 34.91 (10), 25.82 (11), 75.98 (12), 45.36 (13), 42.78 (14), 22.99 (15), 27.59 (16), 48.04 (17), 12.46 (18), 21.73 (19), 35.10 (20), 17.81 (21), 31.16 (22), 30.98 (23), 174.69 (24), 51.60 (OCH₃), 129.55 (o-Ar), 128.67 (m-Ar), 133.24 (p-Ar), 130.80 (subst-Ar), 165.81 (Ar—CO), 95.66 (2′-OTHP), 31.22 (3′-OTHP), 19.73 (4′-OTHP), 25.82 (5′-OTHP), 62.86 (6′-OTHP).

less retained 7alpha-O-[2′(R)]THP isomer 64

¹H NMR (CDCl₃) 0.832 (s, 18-CH₃), 0.864 (d, 21-CH₃), 1.015 (s, 19-CH₃), 3.612 (s, OCH₃), 3.5 & 3.9 (2m, 6′-H, 2H), 3.77 (m, 7-H), 4.66 (m, 2′-H). 5.41 (m, 12-H), 7.43 (m, m-Ar—H, 2H), 7.58 (m, p-Ar—H, 1H), 8.03 (o-Ar—H, 2H).

¹³C NMR (CDCl₃) 36.73 (1), 36.73 (2), 213.59 (3), 45.50 (4), 43.15 (5), 32.98 (6), 77.16 (7), 39.91 (8), 28.64 (9), 34.85 (10), 25.77 (11), 75.87 (12), 45.53 (13), 43.26 (14), 24.15 (15), 27.50 (16), 47.87 (17), 12.47 (18), 21.74 (19), 34.90 (20), 17.77 (21), 31.10 (22), 30.92 (23), 174.68 (24), 51.61 (OCH₃), 129.51 (o-Ar), 128.70 (m-Ar), 133.25 (p-Ar), 130.78 (subst-Ar), 165.68 (Ar—CO), 101.97 (2′-OTHP), 31.47 (3′-OTHP), 20.14 (4′-OTHP), 25.59 (5′-OTHP), 63.08 (6′-OTHP).

### Example 8.4

### Preparation of 3-oxo-7α,12α-dibenzoyloxy-24-methoxycholane (71)

Compound (71) is prepared according to the following reaction scheme:

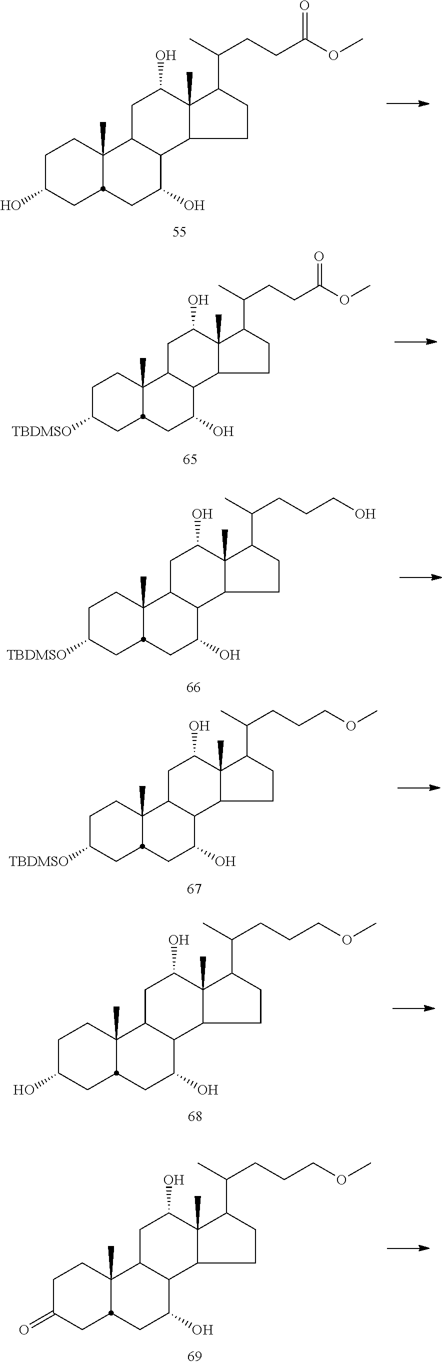

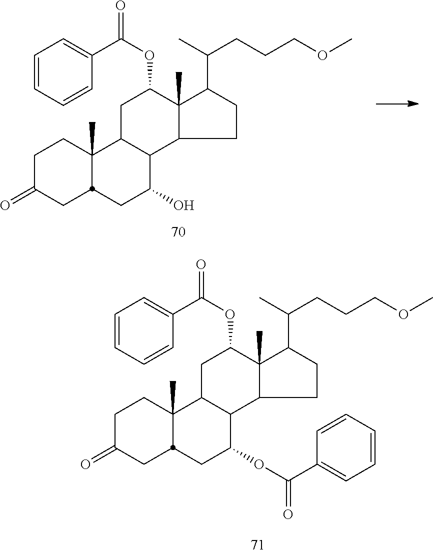

### Methyl 3α-(tert-butyl-dimethyl-silanyloxy)-7α,12α-dihydroxycholanate (65)

Methyl 3alpha,7alpha,12alpha-trihydroxycholanate 55 (4.0 g, 9.5 mmol, 1.0 Eq.) dissolved in dimethylformamide (40 mL) was stirred for 2 h at rt° in the presence of 1H-Imidazole (1.35 g, 19.8 mmol, 2.1 Eq.) and a limited amount of tert-butyl-dimethylsilyl chloride (1.71 g, 11.3 mmol, 1.2 Eq.). After conventional extraction (ethyl acetate) the crude product was purified by column chromatography on silica gel (cyclohexane/ethyl acetate 1:1) to give the mono-3-silyl ether 65 as a white solid.

### 3α-(tert-Butyl-dimethyl-silanyloxy)-7α,12α-dihydroxycholan-24-ol (66)

To a stirred suspension of lithium aluminum hydride (353 mg, 9.31 mmol, 2.0 Eq.) in freshly distilled dry diethyl ether at 0° C. under nitrogen atmosphere was slowly added a solution of the methyl 7alpha,12alpha-dihydroxycholanate derivative 65 (2.5 g, 4.65 mmol, 1.0 Eq.) in dry diethyl ether (80 mL). After stirring for 3 hours at rt°, the reaction mixture was diluted with diethyl ether (50 mL), cooled at 4° C. and treated by dropwise addition of water until total hydrolysis of the excess of lithium aluminum hydride.

After conventional extraction (diethyl ether) the crude extract was purified by column chromatography on silica gel (cyclohexane/ethyl acetate 1:1) to give the 24-ol 66 derivative as a white solid.

### 3α-(tert-Butyl-dimethyl-silanyloxy)-7α,12α-dihydroxy-24-methoxycholane (67)

To a stirred solution of the 7alpha,12alpha-dihydroxycholan-24-ol derivative 66 (1.57 g, 3.08 mmol, 1.0 Eq.) in freshly distilled dry THF (25 mL) under nitrogen atmosphere, was added, portionwise, a commercial 60% suspension of sodium hydride in mineral oil (222 mg, 5.55 mmol, 1.8 Eq. of oil-free hydride). The reaction mixture was stirred for 1 h at rt°. Then, methyl iodide (218 μL, 3.39 mmol, 1.1 Eq.) was added. The reaction mixture was stirred successively for 3 h at rt° and 3 h at 70° C. then cooled at rt°.

After conventional extraction (ethyl acetate) the crude extract was purified by column chromatography on silica gel (cyclohexane/ethyl acetate 6:4) to give the 24-methyl ether 67 (1.39 g) as a pale yellow paste.

### 3α,7α,12α-Trihydroxy-24-methoxycholane (68)

To a solution of 3alpha-tert-butyl-dimethyl-silyl ether 67 (1.67 g, 3.2 mmol, 1.0 Eq.) in anhydrous THF at 0° C. was added a commercial 1 M solution of tetra n-butylammonium fluoride monohydrate in THF (4.80 mL, 4.80 mmol, 1.5 Eq.). After stirring at rt° for 5 h an additional portion of tetra n-butylammonium fluoride solution (1.60 mmol, 0.5 Eq.) and the reaction mixture was stirred for 2 h at 50° C. then cooled down to rt°.

After conventional extraction (ethyl acetate) the crude extract was purified by column chromatography on silica gel (dichloromethane 100% then dichloromethane/methanol 98:2) to give the triol 68 (0.928 g) as a yellow paste.

### 3-Oxo-7α,12α-dihydroxy-24-methoxycholane (69)

The 3alpha,7alpha,12alpha-triol 68 (1.5 g, 3.67 mmol, 1.0 Eq.) dissolved in toluene (300 mL) was magnetically stirred for 4 h under reflux in a Dean-Stark water trap in the presence of 15.2 g of Ag₂CO₃/Celite reagent (vide supra). The reaction mixture was filtered on a column of Celite which was washed with an excess of toluene. The combined filtrates were evaporated under reduced pressure.

The white extract was analyzed by TLC on fluorescent silica gel (dichloromethane/methanol 9:1), showing a major spot above the starting product. This product was purified by column chromatography on silica gel in similar conditions to give the pure mono-3-ketone 69 (1.15 g) as a white solid.

### 3-Oxo-12α-benzoyloxy-7α-hydroxy-24-methoxycholane (70)

The 3-oxo-7alpha,12alpha-diol 69 (400 mg, 0.98 mmol, 1.0 Eq.) was stirred for 24 h at rt° with a limited amount of benzoyl chloride (0.146 mL, 1.25 mmol, 1.28 Eq.) dissolved in a mixture of pyridine (1.5 mL) and dichloromethane (1.5 mL). The reaction mixture was cooled in an ice-bath at 4° C. and stirred for 30 min after addition of 12 mL of ethyl acetate and 12 mL of a saturated aqueous solution of NaHCO₃.

After conventional extraction (ethyl acetate) and elimination of traces of pyridine by azeotropic evaporation in the presence of n-heptane, the dry extract was analyzed by TLC on fluorescent silica gel (cyclohexane/ethyl acetate 1:1) which showed a minor amount of dibenzoylated by-product.

The crude product was purified by column chromatography on silica gel in similar conditions to give the pure mono-12alpha-benzoate 70 from the more eluted fraction (218 mg) and the 7alpha,12alpha-dibenzoate by-product 71 (vide infra) from the less eluted fraction (126 mg).

### 3-oxo-7α,12α-dibenzoyloxy-24-methoxycholane (71)

This dibenzoate 71 was obtained as a by-product of mono-benzoylation of 3-oxo-7alpha,12alpha-dihydroxy-24-methoxycholane (cf preceding step, above).

¹H NMR (CDCl₃) 0.834 (d, 21-CH₃), 0.875 (s, 18-CH₃), 1.101 (s, 19-CH₃), 3.24 (s, OCH₃), 3.21-3.27 (m, 24-CH₂O), 5.31 (m, 7-H), 5.44 (m, 12-H), 7.47 (m, m-Ar—H, 2H), 7.63 (m, p-Ar—H, 1H), 8.03 (o-Ar—H, 2H).

¹³C NMR (CDCl₃) 36.69 (1), 36.39 (2), 212.45 (3), 44.71 (4), 42.15 (5), 31.18 (6), 71.45 (7), 38.56 (8), 30.37 (9), 34.68 (10), 25.88 (11), 76.01 (12), 45.66 (13), 43.75 (14), 23.14 (15), 27.38 (16), 48.24 (17), 12.40 (18), 21.75 (19), 35.07 (20), 17.98 (21), 26.26 (22), 32.12 (23), 73.33 (24), 58.61 (OCH₃), 129.01/50 (o-Ar/7+12), 128.87/92 (m-Ar/7+12), 133.31/34 (p-Ar/7+12), 130.55/67 (subst-Ar/7+12), 165.53/73 (Ar—CO/7+12).

### Example 8.5

### Preparation of 3-oxo-12α-benzoyloxy-7α-O-[2′(S)]tetrahydro-pyranyloxy-24-methoxycholane (73) and 3-oxo-12α-benzoyloxy-7α-O-[2′(R)]tetrahydropyranyloxy-24-methoxycholane (74)

Compounds (73) and (74) are prepared according to the following reaction scheme:

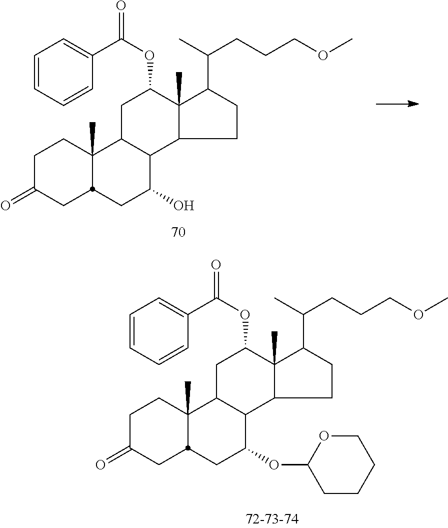

### 3-oxo-12α-benzoyloxy-7α-O-[2′(R+S)]tetrahydropyranyloxy-24-methoxycholane (72); 3-oxo-12α-benzoyloxy-7α-O-[2′(S)]tetrahydropyranyloxy-24-methoxycholane (73) and 3-oxo-12α-benzoyloxy-7αa-O-[2′(13)]tetrahydro-pyranyloxy-24-methoxycholane (74)

The 7alpha-hydroxy derivative 70 (120 mg, 0.23 mmol, 1.0 Eq.) was stirred for 4 days at rt° in an extemporaneously prepared solution containing anhydrous THF (2.5 mL), freshly distilled dihydropyrane (956 μL) and p-toluenesulfonic acid (8 mg). The reaction was stopped by addition of an excess of a saturated NaHCO₃ solution.

After conventional extraction (ethyl acetate) the residue was analyzed by TLC on fluorescent silica gel (petroleum ether/ethyl acetate, 2:1). TLC using cyclohexane/ethyl acetate 9:1 showed two spots with very close Rf values corresponding to the presence of R/S isomers in the crude tetrahydropyranyl ether derivative 72. This product was purified by column chromatography (cyclohexane/ethyl acetate 9:1 to give a mixture (136 mg) of R/S isomers. A part (40 mg) of this mixture of both isomers was separated by preparative TLC (cyclohexane/ethyl acetate 7:3, ×17 developments of 1 cm length each) to provide pure samples of the more retained isomer 73 (10 mg) and of the less retained isomer 74 (10 mg).

more retained 7alpha-O-[2′(S)]THP isomer 73

¹H NMR (CDCl₃) 0.842 (s, 18-CH₃), 0.882 (d, 21-CH₃), 1.036 (s, 19-CH₃), 3.270 (s, OCH₃), 3.26-3.29 (m, 24-CH₂O), 3.5 & 3.9 (2m, 6′-H, 2H), 3.88 (m, 7-H), 4.72 (m, 2′-H), 5.44 (m, 12-H), 7.44 (m, m-Ar—H, 2H), 7.58 (m, p-Ar—H, 1H), 8.08 (o-Ar—H, 2H).

¹³C NMR (CDCl₃) 36.68 (1), 36.64 (2), 213.11 (3), 45.51 (4), 42.78 (5), 29.46 (6), 72.23 (7), 39.61 (8), 28.82 (9), 34.93 (10), 25.86 (11), 76.08 (12), 45.33 (13), 42.82 (14), 23.04 (15), 27.68 (16), 48.24 (17), 12.47 (18), 21.74 (19), 35.41 (20), 18.16 (21), 26.47 (22), 32.29 (23), 73.49 (24), 58.64 (OCH₃), 129.56 (o-Ar), 128.64 (m-Ar), 133.17 (p-Ar), 130.89 (subst-Ar), 165.82 (Ar—CO), 95.60 (2′-OTHP), 31.16 (3′-OTHP), 19.66 (4′-OTHP), 25.80 (5′-OTHP), 62.77 (6′-OTHP).

less retained 7alpha-O-[2′(R)]THP isomer 74

¹H NMR (CDCl₃) 0.831 (s, 18-CH₃), 0.872 (d, 21-CH₃), 1.014 (s, 19-CH₃), 3.286 (s, OCH₃), 3.27-3.29 (m, 24-CH₂O), 3.5 & 3.9 (2m, 6′-H, 2H), 3.76 (m, 7-H), 4.66 (m, 2′-H). 5.42 (m, 12-H), 7.42 (m, m-Ar—H, 2H), 7.57 (m, p-Ar—H, 1H), 8.02 (o-Ar—H, 2H).

¹³C NMR (CDCl₃) 36.71 (1), 36.71 (2), 213.59 (3), 45.49 (4), 43.11 (5), 32.99 (6), 77.20 (7), 39.90 (8), 28.61 (9), 34.84 (10), 25.74 (11), 75.94 (12), 45.46 (13), 43.26 (14), 24.15 (15), 27.55 (16), 48.00 (17), 12.45 (18), 21.72 (19), 35.14 (20), 18.09 (21), 26.28 (22), 32.19 (23), 73.42 (24), 58.64 (OCH₃), 129.49 (o-Ar), 128.65 (m-Ar), 133.15 (p-Ar), 130.83 (subst-Ar), 165.80 (Ar—CO), 101.91 (2′-OTHP), 31.42 (3′-OTHP), 20.09 (4′-OTHP), 25.58 (5′-OTHP), 63.00 (6′-OTHP).

### Example 9

### Preparation of 3,17α-dibenzoyloxypregna-3,5-dien-20-one (75)

Compound (75), which is a BRCP inhibitor, is prepared according to the following reaction scheme:

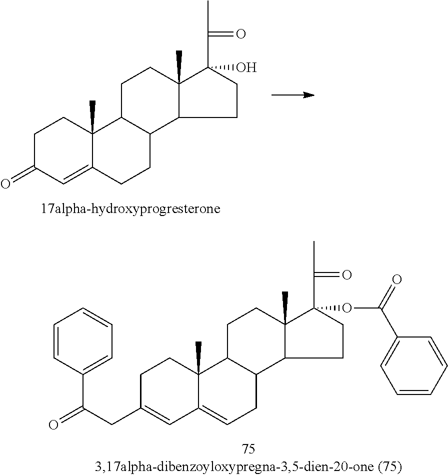

17alpha-hydroxypregna-4-en-3,20-dione (“17alpha-hydroxyprogesterone”) (from Sigma)(320 mg, 0.968 mmol, 1.0 Eq.) was stirred for 12 h under reflux with benzoyl chloride (0.640 mL, 5.513 mmol, 5.7 Eq.) dissolved in pyridine (8 mL) containing dimethylaminopyridine (177 mg, 1.5 Eq.). The reaction mixture was cooled at 4° C. and stirred for 30 min after addition of 5 mL of ethyl acetate and 5 mL of a saturated aqueous solution of NaHCO₃. After conventional extraction (ethyl acetate) and elimination of traces of pyridine by azeotropic evaporation in the presence of n-heptane, the dry product was analyzed by TLC on fluorescent silica gel (petroleum ether/ethyl acetate 2:1).

The crude product was purified by preparative TLC in similar conditions to give a pure sample of enol benzoate derivative 75 (188 mg).

¹H NMR (CDCl₃) 0.778 (s, 18-CH₃), 1.080 (s, 19-CH₃), 2.293 (s, 21-CH₃), 5.462 (d, 6-H), 5.841 (d, 4-H), 7.44/50 (m, m-Ar—H, 4H), 7.57/71 (m, p-Ar—H, 2H), 8.10 (m, o-Ar—H, 4H).

¹³C NMR (CDCl₃) 31.91 (1), 24.92 (2), 147.32 (3), 123.77 (4), 139.42 (5), 117.22 (6), 33.86 (7), 31.70 (8), 50.88 (9), 35.00 (10), 20.68 (11), 30.06 (12), 48.44 (13), 47.57 (14), 24.10 (15), 33.59 (16), 90.03 (17), 15.49 (18), 18.95 (19), 211.88 (20), 28.00 (21), 129.93/130.18 (o-Ar/3+17), 128.47/49 (m-Ar/3+17), 133.29/69 (p-Ar/3+17), nd (superimposed at 130) (subst-Ar/3+17), 165.15/170.90 (Ar—CO/3+17).

### Example 10

### Preparation of 38-(2-Hydroxy-4-azidobenzoyl)amidopropyl-oxypregn-5-en-20-one (77) and 3β-(2-Nitro-5-azidobenzoyl)amidopropyloxy-pregn-5-en-20-one (78)

These compounds are prepared by dioxolanation of 3β-hydroxypregn-5-en-20-one (from Sigma) followed by cyanoethylation of the 3-hydroxy group, reduction of the cyanoethyl group with LiAlH₄ to aminopropylamine, hydrolysis of the protecting ketal group and N-acylation with two corresponding commercially available arylazide derivatives (from Pierce) having a pre-activated carboxylic group (Roy and Ray, Steroids 1995, 60, 530-533).

### Example 11

### Preparation of 7-mono-substituted (5α-H)pregnane-3,20-dione derivatives

### Example 11.1

### Preparation of 7α-(2-Hydroxy-4-azidobenzoyl)amidopropyloxy (5α)pregnane-3,20-dione (79) and 7α-(2-Nitro-5-azidobenzoyl)amidopropyloxy(5α) pregnane-3,20-dione (80)

These compounds are prepared from 3,20-bis-ethylenedioxy-pregn-5-en-7-one (cf above Example 1, compound 1) by catalytic hydrogenation followed by reduction of the 7-ketone with lithium tri-sec-butylborohydride and acidolysis (cf above Example 4, compounds 15-17) thus yielding 7α-hydroxy-(5α) dihydroprogesterone which was converted to arylazide derivatives (79) and (80), as described above for compounds (77) and (78).

### Example 11.2

### Preparation of 7β-Tetrahydropyranyloxy(5α)pregnane-3,20-dione (isomer S) (81), 7β-Tetrahydropyranyloxy(5α)pregnane-3,20-dione (isomer R) (82), 7α-Tetrahydropyranyloxy(5α)pregnane-3,20-dione (isomer S) (83) and 7α-Tetrahydropyranyloxy(5α)pregnane-3,20-dione (isomer R) (84)

These compounds are prepared by tetrahydropyranylation (and TLC separation of R/S isomers) of 7α- or 76-hydroxy-(5α)dihydroprogesterone intermediates obtained in several steps from progesterone protected as a 5-ene-3,20-bis-dioxolane which was converted to a 5-en-7-one by allylic oxidation with CrO₃-(pyridine)₂ complex (Mappus E. & Cuilleron C. Y, J. Chem. Res. 1979 [S], 42-3; [M],501-35), then catalytically hydrogenated to a (5alpha)dihydro-7-one (Mappus E. et Cuilleron C. Y. Steroids, 1979, 33, 693-718) reduced selectively either to 7alpha- or 7-beta-hydroxy isomers (Amann A, Ourisson G, Luu B Synthesis 1987, 1002-4) before final hydrolysis of protecting ketal groups.

### Example 11.3

### Preparation of 7α-(2′-Hydroxy)ethyloxy(5α)pregnan-3,20-dione (85), 7α-(2′-Tetrahydropyranyloxy)ethyloxy(5α)pregnane-3,20-dione (86) and 76-(2′-Tetrahydropyranyloxy)ethyloxy(5α)pregnane-3,20-dione (87)

These compounds are prepared by tetrahydropyranylation (and TLC separation of R/S isomers) of 7α-(2′-hydroxy)ethyloxy(5α)pregnan-3,20-dione (85) obtained in several steps from 7alpha- or 7beta-hydroxy(5alpha)pregnane-3,20-bis-ethyleneketal precursors by O-carboxymethylation with a chloracetate salt in the presence of NaH, esterification to methyl ester, reduction with LiAlH₄ to an oxyethanol followed by hydrolysis of protecting groups.

### Example 11.4

### Preparation of 76-Tetrahydropyranyloxy-3-ethylenedioxy (5α)pregnan-20-one (isomer S) (88) and 76-Tetrahydropyranyloxy-3-ethylenedioxy (5α)pregnan-20-one (isomer R) (89)

These compounds are prepared by tetrahydropyranylation (and TLC separation of R/S isomers) of a 7β-hydroxy-3-ethylenedioxy(5α)pregnan-20-one by-product resulting from partial hydrolysis of the 3,20-bisdioxolane precursor.

### Example 12

### Preparation of 7α-Benzoyl(5β)pregnane-3,20-dione (90)

Compound (90) is prepared by benzoylation of 7α-hydroxy(5β)pregnane-3,20-dione obtained in several steps from a 20-mono-dioxolanated 4,6-dien-3-one precursor by selective 6α,7α-epoxidation with m-chloroperbenzoic acid, catalytic hydrogenation and ketal hydrolysis (Lai et al., Steroids 1983, 42, 707-711).

### Example 13

### Preparation of 7α-phenylpropylcarboxamido-3,20-bisethylenedioxypregn-5-ene (91)

Compound (91) is prepared by acylation of 7alpha-amino-3,20-bisethylenedioxypregn-5-ene obtained via reduction of the oxime derivative from a 5-en-7-one precursor (Mappus et al., Steroids 1981, 38, 607-632).

### Example 14

### Preparation of 7α-(2′-Hydroxy)ethylpregn-4-ene-3,20-dione (92) and 7α-(2′-Tetrahydropyranyloxy)ethylpregn-4-ene-3,20-dione (unseparated R/S isomers) (93)

These compounds are prepared by tetrahydropyranylation of a 7alpha-hydroxyethyl group obtained by LiAlH₄ reduction of a 7alpha-methylene carboxylic methyl ester side-chain synthesized in several steps, according to a reported procedure using malonic alkylation of a 7-bromopregn-5-ene-3,20-bis-ethyleneketal precursor, followed by decarboxylation, esterification and hydrolysis of the 3,20-bisdioxolane protecting group (Duval et al., J. Steroid Biochem. 1985, 22, 67-78).

### Example 15

### Preparation of 11-mono-substituted pregn-4-ene-3,20-dione derivatives

### Example 15.1

### Preparation of 11α-(2-Hydroxy-4-azidobenzoyl)amidopropyl-oxypregn-4-ene-3,20-dione (94) and 11α-(2-Nitro-5-azidobenzoyl)amidopropyloxypregn-4-ene-3,20-dione (95)

These compounds are prepared from 11alpha-hydroxy-3,20-bisethylenedioxypregn-5-ene-3,20-dione as described for compounds (77), (78), (79) and (80).

### Example 15.2

### Preparation of 11α-(2′-Tetrahydropyranyloxy)ethylpregn-4-ene-3,20-dione (96)

This compound is prepared by tetrahydropyranylation (and TLC separation of R/S isomers) of 11alpha-(2′-hydroxy)ethyloxypregn-4-ene-3,20-dione obtained in several steps from 11alpha-hydroxypregn-5-ene-3,20-bis-ethyleneketal as described above for compound 85.

### Example 15.3

### Preparation of 11α-Tetrahydropyranyloxypregn-4-ene-3,20-dione (isomer S) (97) and 11α-Tetrahydropyranyloxypregn-4-ene-3,20-dione (isomer R) (98)

These compounds are prepared by tetrahydropyranylation (and TLC separation of R/S isomers) of 11alpha-hydroxyprogesterone.

### Example 16

### Preparation of 17α-Tetrahydropyranyloxy(5β)pregnan-3,20-dione (less retained isomer)(100) and 17α-Tetrahydropyranyloxy(5β)pregnan-3,20-dione (more retained isomer)(101)

These compounds are prepared by tetrahydropyranylation (and TLC separation of R/S isomers) of 17alpha-hydroxy(5beta)pregnan-3,20-dione obtained by catalytic hydrogenation of 17alpha-hydroxyprogesterone.

### Example 17

### Preparation of 21-Tetrahydropyranyloxypregn-4-ene-3,20-dione (unseparated mixture of R/S isomers (102) and (103))

This compound is prepared by tetrahydropyranylation of 21-[p-(N-α-(+)Methylbenzylaminoacylamino-phenyl)thio]pregn-4-ene-3,20-dione (Leonessa et al., J. Med. Chem. 2002, 45, 390-398).

### Example 18

### Preparation of 3,7-disubstituted (5α-H)pregnane-3,20-dione derivatives

### Example 18.1

### Preparation of 7α-Tetrahydropyranyloxy-3β-benzoyloxy(5α) pregnan-20-one (unseparated mixture of R/S isomers)(104), 7α-Tetrahydropyranyl-oxy-3β-benzoyloxy(5α)pregnan-20-one (isomer S)(105) and 7α-Tetrahydropyranyl-oxy-3β-benzoyloxy(5α)pregnan-20-one (isomer R)(106)

These compounds are prepared by tetrahydropyranylation (and TLC separation of R/S isomers) of 7alpha-hydroxy-3beta-benzoyloxy(5alpha)pregnan-20-one obtained in several steps from 3-benzoyloxy(5alpha)pregn-5-en-20-one as described for compounds (81), (82), (83), (84), (107), (108), (109), (110), (111) and (112).

### Example 18.2

### Preparation of 7β-Tetrahydropyranyloxy-3-β-benzoyloxypregn-5-en-20-one (isomer S)(107), 7β-Tetrahydropyranyloxy-3-β-benzoyloxypregn-5-en-20-one (isomer R)(108), 7α-Tetrahydropyranyloxy-3-β-benzoyloxypregn-5-en-20-one (isomer S)(109) and 7α-Tetrahydropyranyloxy-3-β-benzoyloxypregn-5-en-20-one (isomer R)(110)

These compounds are prepared by benzoylation (and TLC separation of R/S isomers) of 3beta-hydroxy-7alpha-tetrahydropyranyloxypregn-5-en-20-one resulting from hydrolysis of its protected 3-t-butyldimethylsilyl ether precursor, obtained in several steps as described above for compounds (81), (82), (83) and (84).

### Example 19

### Preparation of 7,11-disubstituted (5β-H)pregnane-3,20-dione derivatives

### Example 19.1

### Preparation of 11α-Hydroxy-7β-benzoyloxy(5β)pregnane-3,20-dione (37)

Compound (37) is prepared (cf above) as a precursor for the synthesis of active compounds (39), (40) and (41).

### Example 19.2

### Preparation of 11α,17α-bis-Tetrahydropyranyloxy(5β) pregnane-3,20-dione (53)

Compound (53) is obtained as a less active 17-OTHP(S) isomer of the active compound (51) (cf above).

### Example 20

### Preparation of 11α-Tetrahydropyranyloxy-17α-hydroxy(5β) pregnane-3,20-dione (isomer S) (49) and 11α-Tetrahydropyranyloxy-17α-hydroxy(5β)pregnane-3,20-dione (isomer R) (50)

These compounds are prepared (cf above) as R/S isomeric precursors separated from the mono-11α-tetrahydropyranyloxy derivative (48) in the synthesis of active compounds (51) and (52).

### Example 21

### Preparation of 3,7,20-trisubstituted pregn-5-ene derivatives

### Example 21.1

### Preparation of 7α/β-Hydroxy-pregn-5-ene-3β,20α/β-di-t-butyldimethylsilyl ether (two separated isomers) (111) and (112) and 7α/β-Hydroxypregn-5-ene-3β,20α/β-dibenzoate (mixture of isomers) (113)

These compounds are prepared by reduction of 3β-hydroxypregn-5-en-20-one with LiAlH₄ to a 3,20α/β-diol, conversion to 3,20-di-t-butyldimethylsilyl ether or 3,20-dibenzoate, allylic oxidation to 5-en-7-one with CrO₃-(pyridine)₂ complex and reduction to 7α/β-hydroxy derivatives as described for compounds (81), (82), (83), (84), (107), (108), (109), (110), (111) and (112).

### Example 21.2

### Preparation of 7α/β-Tetrahydropyranyloxypregn-5-ene-3β,20α/β-di-t-butyldimethylsilylether (three separated isomers) (114), (115) and (116)

These compounds are prepared by tetrahydropyranylation (and partial TLC separation of isomers) of 7α/β-hydroxy precursors.

### Example 21.3

### Preparation of 7α/β-Tetrahydropyranyloxy-3β,20α/β-dibenzoyloxy(5β)pregn-5-ene (one separated isomer) (117)

This compound is prepared by tetrahydropyranylation (and partial TLC separation of isomers) of 7alpha/beta-hydroxy precursors.

### Example 22

### Preparation of 3α/β-11α-20α/β-Tribenzoyloxy(5β)pregnane (118)

These compounds are prepared by benzoylation of a mixture of isomeric trihydroxy derivatives resulting from reduction of both 3- and 20-keto groups of a fully 3,20-deprotected by-product obtained as a by-product in the selective 3-ketal hydrolysis of the 11α-hydroxy(5β)pregnane-3,20-bisethyleneketal precursor employed in the synthesis of the active compound 3α/β-11α-dibenzoyloxy (5β)pregnan-20-one (20) (cf above).

### Example 23

### Preparation of 3,7-disubstituted (5β-H)chenodeoxycholane derivatives

### Example 23.1

### Preparation of methyl 3α,7α-bis-tetrahydropyranyloxychenodeoxycholanate (unseparated R/S isomers) CAS [951694-76-1] (119)

This compound is prepared by esterification of chenodeoxycholic acid (purchased from Aldrich) to methyl chenodeoxycholanate, followed by bis-tetrahydropyranylation.

### Example 23.2

### Preparation of methyl 3α-benzoyloxy-7α-tetrahydropyranyloxychenodeoxycholanate (unseparated R/S isomers) (120)

This compound is prepared by 3-monobenzoylation of methyl deoxycholanate (cf above §23.1) and 7-tetrahydropyranylation.

### Example 23.3

### Preparation of methyl 3-oxo-7α-benzoyloxychenodeoxycholanate (121)

This compound is prepared by selective oxidation of the 3alpha-hydroxy group of methyl chenodeoxycholanate (cf above §23.1) to a 3-ketone with silver carbonate reagent (cf above example 8, compound 56) and 7-benzoylation.

### Example 23.4

### Preparation of 3α,7α-bis-tetrahydropyranyloxychenodeoxycholane-24-benzyl ether (unseparated R/S isomers) (122)

This compound is prepared by reduction of methyl 3,7-bis-tetrahydropyranyl-oxychenodeoxydeoxycholanate (cf above §23.1) with LiAlH₄ to a 24-cholanol then converted to a 24-benzylether.

### Example 23.5

### Preparation of 3α-benzoyloxy-7α-tetrahydropyranyloxy-chenodeoxycholane-24-benzyl ether (unseparated R/S isomers) (123)

This compound is prepared by tetrahydropyranylation of the 3-monobenzoate-7-hydroxy precursor obtained by hydrolysis of 3,7-bis-tetrahydropyranyloxy-chenodeoxycholane-24-benzylether (129) to a 3,7-diol followed by selective 3-monobenzoylation.

### Example 23.6

### Preparation of benzyl 3α,7α-bis-tetrahydropyranyloxy-chenodeoxycholanate (unseparated R/S isomers) (124)

This compound is prepared by esterification of chenodeoxycholic acid (from Aldrich) with benzyl chloride and bis-tetrahydropyranylation.

### Example 23.7

### Preparation of benzyl 3α-benzoyloxy-7α-tetrahydropyranyl-oxychenodeoxycholanate (unseparated R/S isomers) (125)

This compound is prepared by 3-monobenzoylation of the benzyl chenodeoxycholanate (cf above §23.6) followed by 7-tetrahydropyranylation.

### Example 24

### Preparation of 3,12-disubstituted (5β-H)deoxycholane derivatives

### Example 24.1

### Preparation of methyl 3α,12α-bis-tetrahydropyranyloxydeoxy-cholanate (unseparated R/S isomers) (126)

Compound (126) is prepared by bis-tetrahydropyranylation of methyl deoxycholanate readily obtained by esterification (MeOH/HCl) of deoxycholic acid (from Aldrich).

### Example 24.2

### Preparation of methyl 3α-benzoyloxy-12α-tetrahydropyranyl-deoxyoxycholanate (separated R/S isomers) (127)

This compound is prepared by 3-monobenzoylation of methyl deoxycholanate and 12-tetrahydropyranylation followed by TLC separation of R/S isomers.

### Example 24.3

### Preparation of methyl 3-oxo-12α-tetrahydropyranyloxy-deoxycholanate (unseparated R/S isomers) (128)

This compound is prepared by selective oxidation of the 3α-hydroxy group of methyl deoxycholanate to a 3-ketone with silver carbonate reagent (cf above §23.1) and 12-tetrahydropyranylation.

### Example 24.4

### Preparation of 3α,12α-bis-tetrahydropyranyloxydeoxycholane-24-benzyl ether (129)

This compound is prepared by reduction of methyl 3,12-bis-tetrahydropyranyloxy-deoxycholanate (cf above §24.1) with LiAlH₄ to the 24-cholanol then converted to a 24-benzylether.

### Example 24.5

### Preparation of 3α-benzoyloxy-12α-tetrahydropyranyl-oxydeoxycholane-24-benzyl ether (separated R/S isomers) (130)

This compound is prepared by tetrahydropyranylation (and TLC separation of R/S isomers) of a 3-monobenzoate-12-hydroxy precursor obtained by hydrolysis of 3,12-bis-tetrahydropyranyloxydeoxycholane 24-benzylether (129) to a 3,12-diol and selective 3-monobenzoylation.

### Example 25

### Preparation of 3,7,12-trisubstituted(5β-H)cholane derivatives

### Example 25.1

### Preparation of methyl 3-α-hydroxy-7α,12α-dibenzoyloxy-cholanate (133)

This compound is prepared by smooth NaBH₄ reduction of methyl 3-oxo-7α,12α-dibenzoyloxycholanate (57).

### Example 25.2

### Preparation of miscellaneous methyl 3-oxo-7α,12α-dihydroxy-cholanate derivatives (134), (135), (136) and (137)

These compounds are prepared: i) by diacylation with p-substituted benzoate groups (i.e. p-nitrobenzoate (134) or p-dimethylaminobenzoate (135)), ii) by monoacylation with an unsubstituted or substituted benzoate followed by etherification with a methoxymethyl group (analogue of a tetrahydropyranyl ether which contributed much less to the inhibiting activity) yielding derivatives such as (136) and iii) by bis-tetrahydropyranylation (forming the compound [176256-19-2] (137)).

### Example 26

### Preparation of 3- or 7-mono-substituted (5α-H)androstane derivatives: 3β-Benzoylamido(5α)androstane [40212-36-0] (138), 3α-Benzoylamido (5α)androstane [40212-36-0] (139), 3β-(N-Methyl)-benzoylamido(5α) androstane (140) and 7β-Benzoyl-amido(5α)androstane (142)

These compounds are prepared from 3- or 7-oxo(5α)androstane precursors via oximation, reduction of oxime to amine, acylation to benzamide and N-methylation.

### Example 27

### Preparation of 7,17-disubstituted (5β-H)androstane derivatives

### Example 27.1

### Preparation of 7α-Benzoyloxy-17β-tetrahydropyranyloxy(5β) androstan-3-one (less retained isomer) (143) and 7α-Benzoyloxy-17β-tetrahydro-pyranyloxy(5β)androstan-3-one (more retained isomer) (144)

These compounds are prepared by benzoylation (and TLC separation of R/S isomers) of 7α-hydroxy-17β-tetrahydropyranyloxy(5β)androstan-3-one obtained in several steps from 17β-hydroxyandrost-4,6-dien-3-one precursor, by tetrahydropyranylation, selective 6α,7α-epoxidation with m-chloroperbenzoic acid and catalytic hydrogenation as described above for compound (90).

### Example 27.2

### Preparation of 7α,17β-Dibenzoyloxy(5β)androstan-3-one (145)

This compound is prepared as described above for 7α-benzoyloxy-17β-tetrahydropyranyloxy(5β)androstan-3-one isomers (143) and (144) but without prior tetrahydropyranylation.

### Example 28

### Preparation of 7,11-disubstituted (5β)pregnane derivatives (205) and (206)

These compounds are prepared via 6,7-epoxypregn-4-en-3-one pathway (Lai et al., Steroids, 1983, 42, 707-711) and have the following formulae:

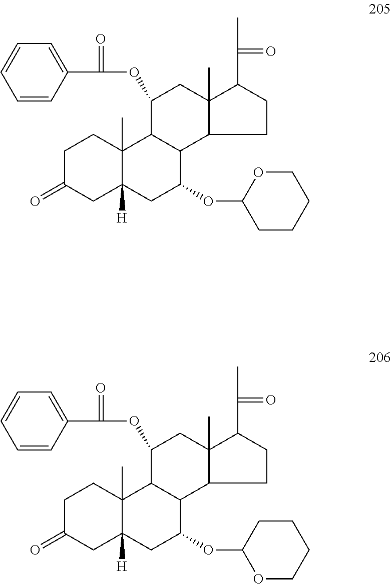

### 11α-(tert-Butyl-dimethyl-silanyloxy)pregn-4-en-3,20-dione (199)

A solution of 11α-hydroxypregn-4-ene-3,20-dione (“11α-hydroxyprogesterone”)(6.0 g, 14.335 mmol, 1.0 Eq.) in anhydrous DMF (240 mL) containing 1H-imidazole (2.37 g, 34.812 mmol, 2.43 Eq.) was cooled at −10° C. (ice-acetone bath). Solid tert-butyldimethylsilyl chloride (5.2 g, 34.499 mmol, 2.40 Eq.) was added under argon atmosphere. The reaction was stirred at rt° for 11 days until completion (monitoring by TLC).

The reaction mixture was extracted by pouring it in 800 mL of water. The solid precipitate of steroid derivative was collected by filtration and washed with water. The solid residue was extracted with ethyl acetate. The organic layer was washed with water, filtered on a phase-separating paper (Whatman), evaporated to dryness under reduced pressure and dried by azeotropic distillation with toluene under reduced pressure.

The dry product (8.55 g) was analyzed by TLC on fluorescent silica gel (petroleum ether/ethyl acetate 5:1).

The crude residue was purified by flash-chromatography on silica gel (230-400 mesh) using petroleum ether/ethyl acetate 6:1 as eluent to give a sample (2.4 g) of pure silyl ether 199 whereas other less pure fractions were recycled.

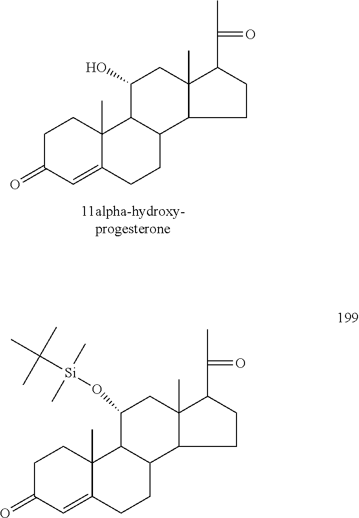

### 11α-(tert-Butyl-dimethyl-silanyloxy)pregn-4,6-dien-3,20-dione (200)

The enone derivative 199 (3.0 g, 6.746 mmol, 1.0 Eq.) was dissolved in hot t-butanol (150 mL) and stirred under reflux for 3.5 h in the presence of tetrachlorobenzoquinone (4.0 g, 16.27 mmol, 2.4 Eq.) until UVmax absorbance of extracted aliquots shifted from 240 to 285 nm.

The reaction mixture was filtered on a slightly heated basic alumina column and evaporated. The dry product (2.8 g) was analyzed by TLC on fluorescent silica gel (petroleum ether/ethyl acetate 3:1) revealing the presence of a slightly more polar minor contaminant.

The colored crude residue was purified by flash-chromatography on silica gel (230-400 mesh) using petroleum ether/ethyl acetate 3:1 as eluent to give a sample (1.16 g) of pure dienone 200 whereas the other fractions were further purified by preparative TLC on fluorescent silica gel (petroleum ether/ethyl acetate 3:1) to give an additional sample (0.68 g) of dienone.

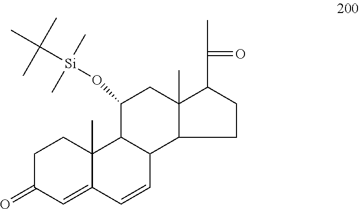

### 6α,7α-Epoxy-11α-(tert-butyl-dimethyl-silanyloxy)pregn-4-ene-3,20-dione (201)

The dienone derivative 200 (682 mg, 1.541 mmol, 1.0 Eq.) was dissolved in dichloromethane (90 mL) and stirred under argon atmosphere for 3 days at rt° in the presence of m-chloroperbenzoic acid (430 mg, 2.492 mmol, 1.62 Eq.). The reaction being still incomplete, an additional amount of m-chloroperbenzoic acid (215 mg, 1.246 mmol, 0.81 Eq) was added and stirring was prolunged for 4 days until starting product was totally transformed.

The reaction mixture, cooled in an ice-bath, was inactivated by addition of an excess of an aqueous 10% sodium sulfite solution (100 mL). The dichloromethane organic layer was decanted, washed with a saturated aqueous solution of sodium bicarbonate and evaporated.

After a further conventional extraction (ethyl acetate) the residue (633 mg) was analyzed by TLC on fluorescent silica gel (petroleum ether/ethyl acetate 3:1).

Preparative TLC in similar conditions gave a pure sample (405 mg) of monoepoxyde 201.

¹H NMR (CDCl₃) 0.075 & 0.091 (d, SiCH₃), 0.688 (s, 18-CH₃), 0.88 (s, SiC—CH₃), 1.17 (s, 19-CH₃), 2.122 (s, 21-CH₃), 3.35 (d, J=3 Hz, 7-H), 3.46 (d, J=4 Hz, 6-H), 4.06-4.12 (m, 11-H), 6.07 (s, 4-H), 7.44 (m, m-Ar—H), 7.56 (m, p-Ar—H), 8.04 (o-Ar—H).

¹³C NMR (CDCl₃) 36.24 (1), 34.33 (2), 198.88 (3), 132.17 (4), 162.67 (5), 53.42 (6), 51.61 (7), 33.63 (8), 47.41 (9), 37.43 (10), 69.60 (11), 50.34 (12), 44.56 (13), 55.11 (14), 24.16 (15), 23.30 (16), 63.21 (17), 14.59 (18), 18.54 (19), 208.67 (20), 31.63 (21), −2.61 & −3.30 (SiCH₃), 18.52 (SiC—CH₃), 26.43 (SiC-CH₃).

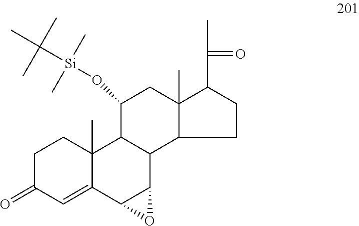

### 7α-Hydroxy-11α-(tert-butyl-dimethyl-silanyloxy)(5β)pregnan-3,20-dione (202)

The mono-epoxide 201 (447 mg, 0.974 mmol, 1.0 Eq.) dissolved in a dioxane-95% ethanol 1:1 v/v mixture containing 0.8% pyridine (45 mL) was introduced in a glass hydrogenation apparatus and magnetically stirred under hydrogen at atmospheric pressure, for 12 h at 30° C., in the presence of 10% Pd—C catalyst (391 mg). The progression of the reaction was monitored by TLC (vide infra) on extracted aliquots.

The reaction mixture was filtered on a pad of ™Celite and evaporated under reduced pressure. The residual pyridine was eliminated by azeotropic distillation in the presence of n-heptane.

The residue (460 mg) was analyzed by TLC on fluorescent silica gel (petroleum ether/ethyl acetate 1:1) which showed a major non-UV-absorbing spot.

The crude product was purified by flash-chromatography on silica gel (230-400 mesh) using petroleum ether/ethyl acetate 2:1 as eluent to give a pure sample (125 mg) of (5beta-H)-7alpha-hydroxy compound 202 and slightly impure fractions (287 mg) which were repurified.

¹H NMR (CDCl₃) 0.084 & 0.092 (d, SiCH₃), 0.622 (s, 18-CH₃), 0.896 (s, SiC—CH₃), 1.087 (s, 19-CH₃), 2.128 (s, 21-CH₃), 3.92 (m broad, 11-H), 4.12 (m, 7-H).

¹³C NMR (CDCl₃) 39.71 (1), 38.88 (2), 214.19 (3), 34.64 (4), 45.31 (5), 46.42 (6), 68.76 (7), 38.80 (8), 40.90 (9), 36.61 (10), 70.72 (11), 50.23 (12), 44.18 (13), 50.48 (14), 24.21 (15), 23.08 (16), 63.65 (17), 14.45 (18), 22.90 (19), 209.03 (20), 31.71 (21), −2.50 & −3.15 (SiCH₃), 18.77 (SiC—CH₃), 26.67 (SiC-CH₃).

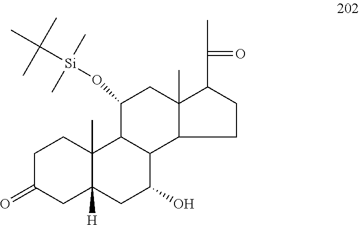

### 7α-O-[2′(R+S)]Tetrahydropyranyloxy-11α-(tert-butyl-dimethyl-silanyloxy)-(5β)pregnane-3,20-dione (203)

The 7α-hydroxy derivative 202 (216 mg, 0.467 mmol, 1.0 Eq.) was stirred for 1 h at rt° then for 12 h at 4° C. in an extemporaneously prepared solution containing anhydrous THF (4.5 mL), freshly distilled dihydropyrane (0.9 mL) and p-toluenesulfonic acid (4.5 mg). The reaction was stopped by addition of an excess of a saturated NaHCO₃ solution.

After conventional extraction (ethyl acetate) the residue (260 mg) was analyzed by TLC on fluorescent silica gel (petroleum ether/ethyl acetate 3:1).

The crude product was purified by flash-chromatography on silica gel (230-400 mesh) using petroleum ether/ethyl acetate 4:1 as eluent to give a pure sample (219 mg) of the unseparated tetrahydropyranyl ether R/S mixture 203.

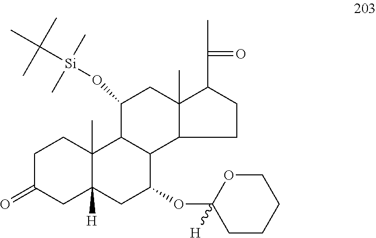

### 7α-O-[2′(R+S)]Tetrahydropyranyloxy-11α-hydroxy(5β)pregnane-3,20-dione (204)

The 11α-tert-butyldimethylsilyl derivative 203 (175 mg, 0.320 mmol, 1.0 Eq.) dissolved in THF (2.4 mL) was stirred for 2 h at 4° C. then for 48 h at rt° after addition of a commercial 1M solution of tetra-butylammonium fluoride (1.147 mL, 1.147 mmol, 0.300 g, 3.59 Eq.) in THF.

After addition of a saturated aqueous NaHCO₃, the reaction mixture was evaporated to dryness under reduced pressure.

After conventional extraction (ethyl acetate) the extract (135 mg) was analyzed by TLC on fluorescent silica gel (petroleum ether/ethyl acetate 1:1 and 2:1).

The crude 11alpha-hydroxy product 204 was employed in the next step without further purification.

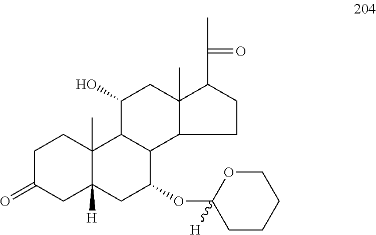

### 7α-O-[2′(R and S)]Tetrahydropyranyloxy-11α-benzoyloxy(5β)pregnane-3,20-dione (205) and (206)

The 11α-hydroxy derivative 204 (135 mg, 0.312 mmol, 1.0 Eq.) was stirred for 12 h at rt° with benzoyl chloride (0.20 mL, 1.723 mmol, 5.52 Eq.) dissolved in pyridine (5.5 mL).

The reaction mixture was cooled at 4° C., stirred for 30 min after addition of 25 mL of ethyl acetate and 25 mL of a saturated aqueous NaHCO₃ solution.

After conventional extraction (ethyl acetate) and elimination of traces of pyridine by azeotropic evaporation in the presence of n-heptane, the dry residue was analyzed by TLC on fluorescent silica gel (petroleum ether/ethyl acetate 1:1; petroleum ether/MTBE 1:1).

The crude product was purified by preparative TLC on fluorescent silica gel (petroleum ether/ethyl acetate 4:1, ×9 developments) which gave a less polar fraction (45 mg), a more polar fraction (17 mg) and an intermediate fraction containing a mixture of these two fractions (49 mg) which was further purified by a similar preparative TLC (petroleum ether/ethyl MTBE 2:1, ×7 developments) leading to two similarly enriched less- and more polar fractions (13 mg and 27 mg). A final preparative TLC (petroleum ether/ethyl MTBE 2:1, ×8 developments) of similar fractions gave pure samples of the less polar product (47 mg) and of the more polar product (30 mg) shown (RX data) to correspond respectively the R and S tetrahydropyranyl ether isomers 205 and 206.

(more-retained) 7α-O-[2′(S)]tetrahydropyranyloxy isomer (206) (structure confirmed by X-ray crystallographic data)

¹H NMR (CDCl₃) 0.757 (s, 18-CH₃), 1.166 (s, 19-CH₃), 2.095 (s, 21-CH₃), 3.5 & 3.9 (2m, 6′-H, 2H), 3.9 (m superimposed to 6′-H, 7-H), 4.58 (d, J=5.5 Hz, 2′-H), 5.56 (m, 11-H), 7.44 (m, m-Ar—H), 7.57 (m, p-Ar—H), 8.01 (o-Ar—H).

¹³C NMR (CDCl₃) 39.32 (1), 38.34 (2), 213.05 (3), 45.25 (4), 44.47 (5), 29.72 (6), 72.46* (7), 38.02 (8), 39.27 (9), 36.72 (10), 72.44* (11), 46.11 (12), 44.10 (13), 49.92 (14), 23.77 (15), 23.31 (16), 63.37 (17), 14.36 (18), 22.97 (19), 209.16 (20), 31.93 (21), 129.85 (o-Ar), 128.84 (m-Ar), 133.56 (p-Ar), 130.54 (subst-Ar), 166.14 (Ar—CO), 97.40 (2′-OTHP), 31.67 (3′-OTHP), 21.16 (4′-OTHP), 25.66 (5′-OTHP), 64.58 (6′-OTHP).

(less-retained) 7α-O-[2′(R)]tetrahydroovranyloxy isomer (205) (structure confirmed by X-ray crystallographic data)

¹H NMR (CDCl₃) 0.754 (s, 18-CH₃), 1.147 (s, 19-CH₃), 2.097 (s, 21-CH₃), 3.5 & 3.9 (2m, 6′-H, 2H), 3.74 (s broad, 7-H), 4.54 (d, J=5.0 Hz, 2′-H), 5.56 (m, 11-H), 7.42 (m, m-Ar—H), 7.56 (m, p-Ar—H), 8.00 (o-Ar—H).

¹³C NMR (CDCl₃) 39.38 (1), 38.43 (2), 213.71 (3), 45.23 (4), 44.83 (5), 32.93 (6), 77.20 (7), 38.10 (8), 39.58 (9), 36.59 (10), 72.38 (11), 46.12 (12), 44.17 (13), 50.34 (14), 24.83 (15), 23.28 (16), 63.30 (17), 14.35 (18), 22.95 (19), 208.83 (20), 31.92 (21), 129.85 (o-Ar), 128.84 (m-Ar), 133.58 (p-Ar), 130.51 (subst-Ar), 166.14 (Ar—CO), 102.82 (2′-OTHP), 31.77 (3′-OTHP), 21.00 (4′-OTHP), 25.64 (5′-OTHP), 64.21 (6′-OTHP).

### Example 29

### Preparation of 6α-substituted derivatives of methyl 3-oxohydrodeoxycholate (210) and (211)

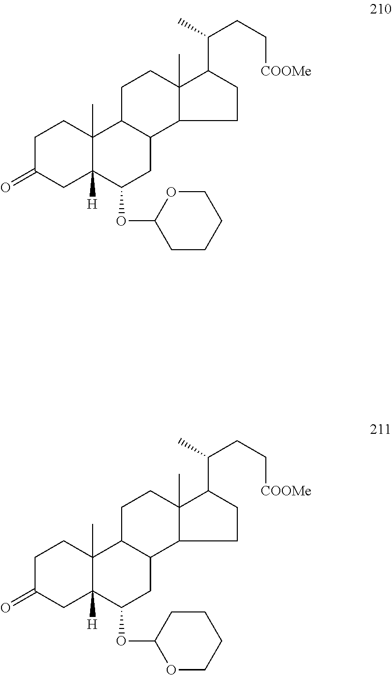

### Methyl 3-oxo-6α-hydroxycholanate (207)

Methyl 3α,6α-dihydroxycholanate (“methyl hyodeoxycholate”) (200 mg, 0.492 mmol, 1.0 Eq.) dissolved in toluene (40 mL) was magnetically stirred for 12 h under reflux in a Dean-Stark water trap in the presence of 4.0 g of Ag₂CO₃/Celite reagent (cf Fieser L., Reagents for Organic Synthesis, Vol 2, p. 363; Fétizon M., Balogh V., Golfier M., J. Org. Chem. (1971), 36, 1339-41 and C. Y., Cuilleron, These de Doctorat d'Etat 1971, Orsay-Paris 11).

The reaction mixture was filtered on a column of Celite which was washed with an excess of toluene. The combined filtrates were evaporated under reduced pressure.

The white extract was analyzed by TLC on fluorescent silica gel (petroleum ether/ethyl acetate 1:1), showing a major spot above the starting product. This mono-3-oxo product 207 was employed without further purification in the following next step.

¹H NMR (CDCl₃) 0.673 (s, 18-CH₃), 0.917 (d, 21-CH₃, J=7 Hz), 1.000 (s, 19-CH₃), 3.659 (s, OCH₃), 4.10 (m, 6-H).

¹³C NMR (CDCl₃) 37.41* (1), 37.41*(2), 213.00 (3), 36.37 (4), 50.50 (5), 68.03 (6), 34.71 (7), 34.88 (8), 40.57 (9), 36.56 (10), 21.42 (11), 40.15 (12), 43.18 (13), 56.44 (14), 24.48 (15), 28.41 (16), 56.25 (17), 12.38 (18), 23.19 (19), 35.65 (20), 18.60 (21), 31.37 (22), 31.26 (23), 175.05 (24), 51.86 (OCH₃).

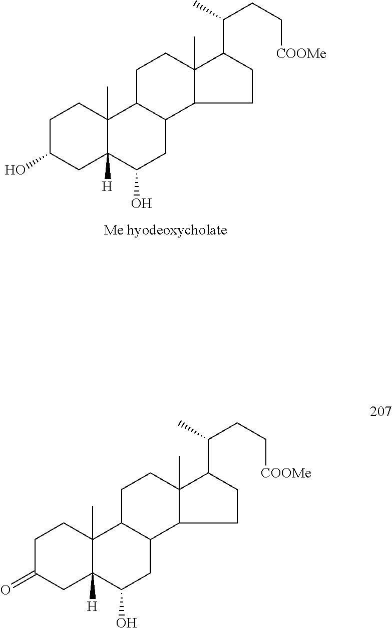

### Methyl 3-oxo-6α-benzoyloxycholanate (208)

The 6α-hydroxy derivative 207 (80 mg, 0.198 mmol, 1.0 Eq.) was stirred for 12 h at rt° with an excess of benzoyl chloride (0.14 mL, 1.206 mmol, 6.1 Eq.) dissolved in a mixture of pyridine (2.2 mL). The reaction mixture was cooled in an ice-bath at 4° C. and stirred for 30 min after addition of 9 mL of ethyl acetate and 9 mL of a saturated aqueous NaHCO₃ solution.

After conventional extraction (ethyl acetate) and elimination of traces of pyridine by azeotropic evaporation in the presence of n-heptane, the crude extract was analyzed by TLC on fluorescent silica gel (petroleum ether/ethyl acetate 3:1) which showed a minor amount of monobenzoylated by-product.

The crude product was purified by preparative TLC in similar conditions to give a pure sample (69 mg) of benzoate 208.

¹H NMR (CDCl₃) 0.704 (s, 18-CH₃), 0.929 (d, 21-CH₃, J=7 Hz), 1.127 (s, 19-CH₃), 3.660 (s, OCH₃), 5.43 (m, 6-H), 7.42 (m, m-Ar—H, 2H), 7.54 (m, p-Ar—H, 1H), 7.98 (o-Ar—H, 2H).

¹³C NMR (CDCl₃) 37.43 (1), 37.25* (2), 212.27 (3), 37.04* (4), 47.60 (5), 71.51 (6), 31.37 (7), 34.81 (8), 40.71 (9), 36.75 (10), 21.39 (11), 40.12 (12), 43.24 (13), 56.51 (14), 24.43 (15), 28.41 (16), 56.25 (17), 12.39 (18), 23.06 (19), 35.65 (20), 18.61 (21), 31.34 (22), 31.26 (23), 175.00 (24), 51.85 (OCH₃), 129.88 (o-Ar), 128.71 (m-Ar), 133.36 (p-Ar), 130.62 (subst-Ar), 166.04 (Ar—CO).

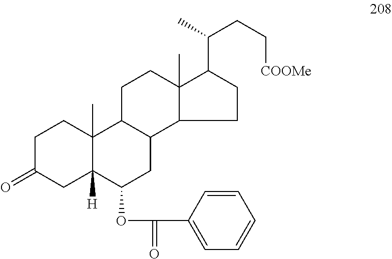

### Methyl 3-oxo-6α-O-[2′(R+S)]tetrahydropyranyloxycholanate (209) and separation of R IS isomers (210) and (211)

The 6α-hydroxy derivative 207 (150 mg, 0.371 mmol, 1.0 Eq.) was stirred for 12 h at rt° with a limited amount of an extemporaneously prepared solution containing anhydrous THF (4 mL), freshly distilled dihydropyrane (0.80 mL 8.768 mmol, 23.6 Eq.) and p-toluenesulfonic acid (0.4 mg). The reaction was stopped by addition of an excess of a saturated aqueous NaHCO₃ solution.

After conventional extraction (ethyl acetate) the residue was analyzed by TLC on fluorescent silica gel (petroleum ether/ethyl acetate 3:1). TLC using petroleum ether/ethyl acetate 6:1 (×6 developments) or petroleum ether/MTBE 4:1 (×6 developments) showed two spots with very close Rf values corresponding to the presence of R/S isomers in the tetrahydropyranylether derivative 209.

These two isomers were fully separated by two successive preparative TLCs in similar conditions to give pure samples of the less polar product 210 (28 mg) and of the more polar product 211 (57 mg) each corresponding to one of the R and S tetrahydropyranyl ether isomers. Current work is aimed at establishing the absolute configurations of these isomers.

(less retained) 6α-O-[2′(S or R)]THP isomer 210

¹H NMR (CDCl₃) 0.662 (s, 18-CH₃), 0.912 (d, 21-CH3, J=7 Hz), 1.007 (s, 19-CH₃), 3.654 (s, OCH₃), 3.5 & 3.8 (2m, 6′-H, 2H), 4.01 (m, 6-H), 4.69 (m, 2′-H).

¹³C NMR (CDCl₃) 37.40 (1), 37.32* (2), 213.54 (3), 37.28* (4), 49.75 (5), 71.79 (6), 31.34* (7), 34.75 (8), 40.81 (9), 36.46 (10), 21.45 (11), 40.16 (12), 43.19 (13), 56.59 (14), 24.49 (15), 28.41 (16), 56.23 (17), 12.36 (18), 23.26 (19), 35.65 (20), 18.60 (21), 31.34** (22), 31.27** (23), 175.02 (24), 51.83 (OCH₃), 96.69 (2′-OTHP), 31.27** (3′-OTHP), 19.65 (4′-OTHP), 25.84 (5′-OTHP), 62.52 (6′-OTHP).

(more retained) 6α-O-[2′(R or S)]THP isomer 211

¹H NMR (CDCl₃) 0.664 (s, 18-CH3), 0.913 (d, 21-CH₃, J=7 Hz), 0.996 (s, 19-CH₃), 3.655 (s, OCH₃), 3.5 & 3.9 (2m, 6′-H, 2H), 4.04 (m, 6-H), 4.59 (m, 2′-H).

¹³C NMR (CDCl₃) 37.44* (1), 37.62* (2), 213.42 (3), 37.10* (4), 47.17 (5), 71.88 (6), 33.19* (7), 34.91 (8), 40.68 (9), 36.39 (10), 21.47 (11), 40.17 (12), 43.18 (13), 56.60 (14), 24.47 (15), 28.43 (16), 56.22 (17), 12.37 (18), 23.22 (19), 35.64 (20), 18.61 (21), 31.35** (22), 31.27** (23), 175.02 (24), 51.83 (OCH₃), 96.65 (2′-OTHP), 31.44** (3′-OTHP), 20.20 (4′-OTHP), 25.77 (5′-OTHP), 63.28 (6′-OTHP).

### Example 30

### Preparation of 6β-substituted derivatives of (5β)pregnane (217) and (218)

These compounds are prepared via hydroboration of pregn-5-ene precursors and have the following formulae:

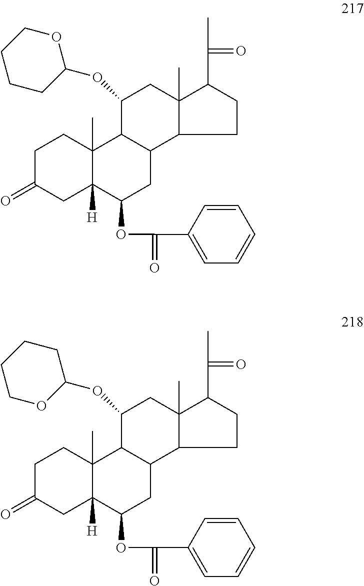

### 6β-Hydroxy-11α-(tert-butyl-dimethyl-silanyloxy(5β)(pregnan-3,20-bisethyleneketal (212)

To a solution of pregn-5-ene derivative 22 (200 mg, 0.375 mmol, 1.0 Eq.) in anhydrous THF (8.0 mL) stirred at 4° C. under argon atmosphere was added, dropwise, a commercial 1 M solution of borane-THF complex in THF (0.65 mL, 0.650 mmol, 1.69 Eq.). The reaction mixture was stirred for 1 h at 4° C. then 1 h at rt°.

The excess of borane was destroyed at 4° C. by dropwise addition of water (1.5 mL), then 2N NaOH solution (1.5 mL) and 30% H₂O₂ (1.5 mL). The reaction mixture was heated for 1 h at 50° C. and evaporated to dryness.

After conventional extraction (MTBE) the extract was analyzed by TLC on fluorescent silica gel (petroleum ether/ethyl acetate 1:1; petroleum ether/MTBE 1:2).

The residue (185 mg) was purified by flash-chromatography on silica gel (230-400 mesh) using petroleum ether/MTBE 1:2 as eluent to give a major fraction containing a nearly pure major product (138 mg) which was submitted to a similar chromatography using petroleum ether/MTBE 1:1 as eluent to give a pure sample (95 mg) of 6-hydroxy compound 212.

¹H NMR (CDCl₃) 0.048 & 0.059 (d, SiCH₃), 0.767 (s, 18-CH₃), 0.867 (s, SiC—CH₃), 1.182 (s, 19-CH₃), 1.287 (s, 21-CH₃), 3.70 (s broad, 6-H), 3.927 (s, 3-OCH₂), 3.84-3.99 (m, 20-OCH₂), 4.00-4.06 (m, 11-H).

¹³C NMR (CDCl₃) 36.35 (1), 31.15 (2), 110.01 (3), 37.26 (4), 49.75 (5), 72.69 (6), 34.57 (7), 29.37 (8), 47.43 (9), 35.84 (10), 70.56 (11), 51.26 (12), 42.69 (13), 55.84 (14), 24.14 (15), 23.20 (16), 58.40 (17), 14.43 (18), 24.84 (19), 111.99 (20), 24.64* (21), 64.53 (3-OCH₂), 63.64 & 65.58 (20-OCH₂), −2.78 & −3.07 (SiCH₃), 18.69 (SiC—CH₃), 26.64* (SiC-CH₃).

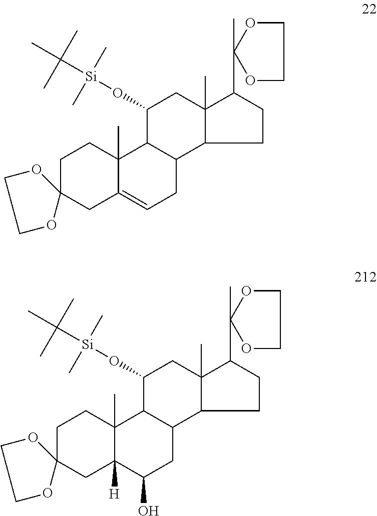

### 6β-Benzoyloxy-11α-tert-butyldimethylsilanyloxy(5beta)pregnan-3,20-bis-ethyleneketal (213)

The 6-hydroxy derivative 212 (397 mg, 0.721 mmol, 1.0 Eq.) was stirred for 12 h at rt° with benzoyl chloride (1 mL, 1.723 mmol, 11.9 Eq.) dissolved in pyridine (15 mL).

The reaction mixture was cooled at 4° C., stirred for 1 h after addition of 60 mL of ethyl acetate and 65 mL of a saturated aqueous NaHCO₃ solution.

After conventional extraction (ethyl acetate) and elimination of traces of pyridine by azeotropic evaporation in the presence of n-heptane, the dry residue was analyzed by TLC on fluorescent silica gel (petroleum ether/ethyl acetate 3:1; petroleum ether/MTBE 1:1).

The crude product (467 mg) was purified by preparative TLC on fluorescent silica gel (petroleum ether/ethyl acetate 4:1) to give a pure sample (451 mg) of benzoate derivative 213.

¹H NMR (CDCl₃) 0.063 & 0.076 (d, SiCH₃), 0.791 (s, 18-CH₃), 0.882 (s, SiC—CH₃), 1.187 (s, 19-CH₃), 1.291 (d, J=2 Hz, 21-CH₃), 3.94 (s, 3-OCH₂), 3.84-3.99 (m, 20-OCH₂), 4.04-4.09 (m, 11-H), 4.969 (s broad, 6-H), 7.44 (m, m-Ar—H), 7.56 (m, p-Ar—H), 8.04 (o-Ar—H).

¹³C NMR (CDCl₃) 36.09 (1), 31.43 (2), 109.76 (3), 37.10 (4), 46.85 (5), 75.48 (6), 31.79 (7), 30.21 (8), 46.66 (9), 35.78 (10), 70.52 (11), 51.30 (12), 42.69 (13), 55.73 (14), 24.13 (15), 23.19 (16), 58.36 (17), 14.52 (18), 24.85 (19), 111.94 (20), 25.88 (21), 64.59 & 64.63 (3-OCH₂), 63.65 & 65.57 (20-OCH₂), −2.73 & −3.09 (SiCH₃), 18.71 (SiC—CH₃), 26.65 (SiC—CH₃), 129.91 (o-Ar), 128.71 (m-Ar), 133.09 (p-Ar), 131.23 (subst-Ar), 166.26 (Ar—CO).

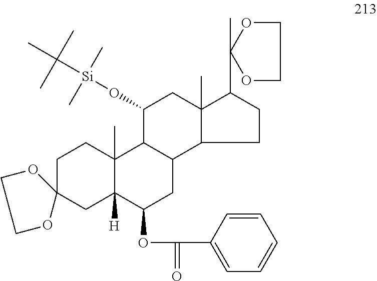

### 6β-Benzoyloxy-11α-hydroxy-(5β)(pregnan-3,20-bis-ethyleneketal (214)

The 11α-tert-butyldimethylsilyl derivative 213 (425 mg, 0.649 mmol, 1.0 Eq.) dissolved in THF (4.80 mL) was stirred for 2 h at 4° C. then for 5 days at rt° after addition of a commercial 1 M solution of tetra-butylammonium fluoride (2.103 mL, 2.103 mmol, 3.24 Eq.) in THF. The reaction being still incomplete, a further amount of tetra-butylammonium fluoride solution was added (1.05 mL, 1.05 mmol, 1.6 Eq.) and the reaction mixture was stirred again for 1 h at 4° C. then for 5 days at rt° until complete hydrolysis.

After addition of a saturated aqueous solution of sodium bicarbonate, the reaction mixture was evaporated to dryness under reduced pressure.

After conventional extraction (ethyl acetate) the dry extract (351 mg) was analyzed by TLC on fluorescent silica gel (petroleum ether/ethyl acetate 1:3).

The crude 11alpha-hydroxy product 214 was employed in the next step without purification.

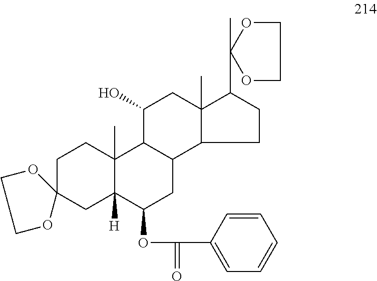

### 6β Benzoyloxy-11α-hydroxy(5β)pregnan-3,20-dione (215)

A solution of bis-ethylenedioxy-11α-ol derivative 214 (351 mg, 0.649 mmol, 1.0 Eq.) in acetone (48 mL) containing p-toluenesulfonic acid monohydrate (80 mg) and a small amount of water (2.4 mL) was stirred for 2 days at rt°. The reaction was quenched with a cold saturated aqueous NaHCO₃ solution and the solvent was evaporated under reduced pressure.

After conventional extraction (ethyl acetate) the dry product (301 mg) was analyzed by TLC on fluorescent silica gel (petroleum ether/ethyl acetate 1:2).

The crude product was purified by preparative TLC on fluorescent silica gel (petroleum ether/ethyl acetate 1:3) to give a pure sample (227 mg) of 3,20-dione 215.

¹H NMR (CDCl₃) 0.710 (s, 18-CH₃), 1.183 (s, 19-CH₃), 2.143 (s, 21-CH₃), 4.09 (m, 11-H), 4.94 (d, J=2 Hz, 6-H), 7.46 (m, m-Ar—H), 7.58 (m, p-Ar—H), 8.01 (o-Ar—H).

¹³C NMR (CDCl₃) 40.03 (1), 38.02 (2), 211.74 (3), 41.99* (4), 48.71 (5), 74.14 (6), 31.36* (7), 30.56 (8), 47.76 (9), 36.01 (10), 68.68 (11), 50.64 (12), 44.56 (13), 55.60 (14), 24.67 (15), 23.38 (16), 63.46 (17), 14.85 (18), 25.24 (19), 209.12 (20), 31.72 (21), 129.88 (o-Ar), 128.84 (m-Ar), 133.42 (p-Ar), 130.69 (subst-Ar), 166.07 (Ar—CO).

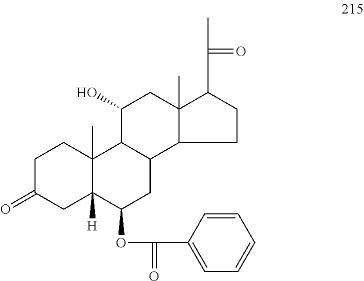

### 6β,11α-Dibenzoyloxy(5β)pregnan-3,20-dione (216)

The 11α-hydroxy derivative 216 (60 mg, 0.133 mmol, 1.0 Eq.) was stirred for 12 h at rt° with benzoyl chloride (0.10 mL, 0.861 mmol, 6.5 Eq.) dissolved in pyridine (2.4 mL).

The reaction mixture was cooled at 4° C., stirred for 1 h after addition of 10 mL of ethyl acetate and 10 mL of a saturated aqueous NaHCO₃ solution.

After conventional extraction (ethyl acetate) and elimination of traces of pyridine by azeotropic evaporation in the presence of n-heptane, the dry residue (0.078 g) was analyzed by TLC on fluorescent silica gel (petroleum ether/ethyl acetate 1:1).

The crude product was purified by preparative TLC on fluorescent silica gel (petroleum ether/ethyl acetate 2:1) to give a pure sample (60 mg) of dibenzoate derivative 216.

¹H NMR (CDCl₃) 0.837 (s, 18-CH₃), 1.186 (s, 19-CH₃), 2.103 (s, 21-CH₃), 4.98 (d, J=2 Hz, 6-H), 5.59-5.65 (m, 11-H), 7.46 (m, m-Ar—H), 7.58 (m, p-Ar—H), 8.01 (o-Ar—H).

¹³C NMR (CDCl₃) 39.34 (1), 37.72 (2), 210.49 (3), 41.92* (4), 48.26 (5), 73.72 (6), 31.29* (7), 30.83 (8), 45.11 (9), 35.96 (10), 71.88 (11), 45.80 (12), 44.41 (13), 55.62 (14), 24.66 (15), 23.27 (16), 63.41 (17), 14.69 (18), 25.23 (19), 208.61 (20), 31.79 (21), 129.89 & 129.79 (o-Ar), 128.92 (m-Ar), 133.55 & 133.71 (p-Ar), 130.52 & 130.41 (subst-Ar), 166.05 & 165.89 (Ar—CO).

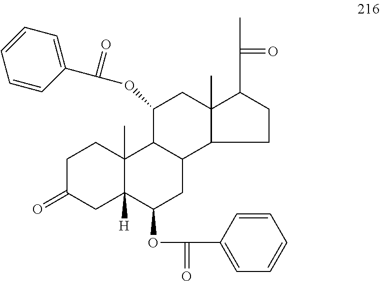

### 6β-Benzoyloxy-11α-O-[2′(R and S)]tetrahydropyranyloxy(5β)pregnan-3,20-dione (217) (S isomer) and (218) (R isomer)

The 11α-hydroxy derivative 215 (147 mg, 0.325 mmol, 1.0 Eq.) was stirred for 1 h at rt° then for 12 h at 4° C. in an extemporaneously prepared solution containing anhydrous THF (3 mL), freshly distilled dihydropyrane (0.6 mL) and p-toluenesulfonic acid (3 mg). The reaction was stopped by addition of an excess of a saturated aqueous NaHCO₃ solution.

After conventional extraction (ethyl acetate) the residue (230 mg) was analyzed by TLC on fluorescent silica gel (petroleum ether/ethyl acetate 2:1).

The crude product was purified by preparative TLC on fluorescent silica gel (petroleum ether/ethyl acetate 2:1) to give two fractions corresponding to pure samples of the less-polar product 217 (89 mg) and of the more-polar product 218 (65 mg) assigned respectively (NMR data) to S and R tetrahydropyranyl ether isomers.

less retained 11α-O-[2′(S)]tetrahydropyranyloxy isomer 217

¹H NMR (CDCl₃) 0.707 (s, 18-CH₃), 1.181 (s, 19-CH₃), 2.152 (s, 21-CH₃), 3.4 & 3.9 (2m, 6′-H, 2H), 4.20-4.24 (m, 11-H), 4.58 (m, 2′-H), 4.92 (d, J=2 Hz, 6-H), 7.45 (m, m-Ar—H), 7.56 (m, p-Ar—H), 8.01 (o-Ar—H).

¹³C NMR (CDCl₃) 39.20 (1), 38.04 (2), 212.20 (3), 42.10* (4), 49.15 (5), 74.22 (6), 31.58* (7), 30.70 (8), 45.77 (9), 36.08 (10), 70.56 (11), 43.89 (12), 44.11 (13), 55.60 (14), 24.78 (15), 23.63 (16), 63.48 (17), 14.78 (18), 24.96 (19), 209.41 (20), 31.83 (21), 129.90 (o-Ar), 128.80 (m-Ar), 133.34 (p-Ar), 130.78 (subst-Ar), 166.08 (Ar—CO), 96.89 (2′-OTHP), 32.26 (3′-OTHP), 22.13 (4′-OTHP), 25.65 (5′-OTHP), 65.93 (6′-OTHP).

more retained 11α-O-[2′(R)]tetrahydropyranyloxy isomer 218

¹H NMR (CDCl₃) 0.669 (s, 18-CH₃), 1.182 (s, 19-CH₃), 2.138 (s, 21-CH₃), 3.5 & 3.9 (2m, 6′-H, 2H), 3.87-3.96 (m, 11-H), 4.68 (m, 2′-H), 4.92 (d, J=2 Hz, 6-H), 7.46 (m, m-Ar—H), 7.57 (m, p-Ar—H), 8.01 (o-Ar—H).

¹³C NMR (CDCl₃) 39.77 (1), 37.95 (2), 211.32 (3), 41.97* (4), 48.89 (5), 74.08 (6), 31.67° (7), 31.03 (8), 45.91 (9), 36.11 (10), 78.45 (11), 48.03 (12), 44.32 (13), 55.25 (14), 24.85 (15), 23.14 (16), 63.69 (17), 14.71 (18), 25.64** (19), 209.11 (20), 31.88 (21), 129.88 (o-Ar), 128.84 (m-Ar), 133.42 (p-Ar), 130.70 (subst-Ar), 166.04 (Ar—CO), 101.68 (2′-OTHP), 31.77° (3′-OTHP), 20.34 (4′-OTHP), 25.64** (5′-OTHP), 63.35 (6′-OTHP).

Biological Results

Protocols

1. Cell Lines and Culture Conditions

The human ACC cell line, NCI-H295R, established from an invasive primary adrenocortical carcinoma has maintained multiple pathways of steroidogenesis (A. F. Gazdar et al., Cancer Research 50 (1990) 5488-5496). It was kindly provided by Dr. Martine Begeot, (INSERM U864, Lyon, France). The human K562 cell line, established from a patient with chronic myelogenous leukemia (C. B. Lozzio and B. B. Lozzio, Blood 45 (1975) 331-334) and the resistant R7 cell line, obtained by treatment of K562 cells with doxorubicine, were kindly provided by Pr. Charles Dumontet (INSERM U590, Lyon, France).

NCI H295R cells were grown in 75-cm² culture flasks at 37° C. in a 5% CO₂ atmosphere. The culture medium consisted of a 1:1 mixture of DMEM and Ham's F-12 medium, supplemented with L-glutamine (2 mM), antibiotics (50 μg/mL streptomycin, 50 U/mL penicillin) and 2% Ultroser G, Ultroser SF (Biorad) and a commercial mixture of insulin, transferin and sodium selenite (ITS+1, Sigma). Cells were harvested with trypsin (0.05%)-EDTA (0.02%) and resuspended in culture medium. Cell viability always exceeded 95%.

The K562/R7 cells were cultured in RPMI 1640 supplemented with 10% foetal calf serum (FCS), L-glutamine (2 mM), glucose (0.3%), sodium pyruvate (1 mM), penicillin (200 U/mL), and streptomycin (100 μg/mL). Cells were maintained at 37° C. in a 5% CO₂ atmosphere.

Media and supplements were obtained from Invitrogen-Gibco (Paisley, UK), culture flasks from BD-Falcon (Meylan, France).

2. Isolation of RNA, and Quantitative RT-PCR

Total RNA was extracted (from H295R and K562/R7 cells lines) using a commercially available kit (RNeasy Mini kit; Qiagen SA, Courtaboeuf, France). First-strand cDNAs were first synthesized from 5 μg of total RNA in the presence of 50 units of the Superscript™ II reverse transcriptase using both random hexamers and oligo (dT) primers (Invitrogen Kit, Life Technologies). Quantitative PCR was performed in a final volume of 20 μl containing 5 μl of a 60-fold dilution of the RT reaction medium, 15 μl of reaction buffer from the FastStart DNA Master Plus SYBER Green Kit (Roche Diagnostics, Basel, Switzerland), and 10 pmol of the specific forward and reverse primers (Operon Biotechnologies, Cologne, Germany) (Table 1).

Standard curves were prepared for each target and reference gene. Each assay was performed in duplicate, and validation of the real-time PCR runs was assessed by evaluation of the melting temperature of the products and by the slope and error obtained with the standard curve. The analyses were performed using Light-Cycler software (Roche Diagnostics). Results are expressed as relative levels after normalization by G3PDH mRNA.

TABLE 1
Specific oligonucleotide PCR primers employed
for the quantification of mRNA of ABC transporters

| | 5′-3′ primers | 3′-5′ primers |
|---|---|---|
| MDR1/ABCB1 | CCCATCATTGCAATAGCAGG | TGTTCAAACTTCTGCTCCTGA |
| | (SEQ ID NO: 1) | (SEQ ID NO: 5) |
| MRP1/ABCC1 | ATGTCACGTGGAATACCAGC | GAAGACTGAACTCCCTTCCT |
| | (SEQ ID NO: 2) | (SEQ ID NO: 6) |
| MRP2/ABCC2 | ACAGAGGCTGGTGGCAACC | ACCATTACCTTGTCACTGTCCATGA |
| | (SEQ ID NO: 3) | (SEQ ID NO: 7) |
| BCRP/ABCG2 | AGATGGGTTTCCAAGCGTTCAT | CCAGTCCCAGTACGACTGTGACA |
| | (SEQ ID NO: 4) | (SEQ ID NO: 8) |

Quantitative RT-PCR experiments showed the presence of MDR1 mRNA in both naturally resistant H295R and doxorubicin-resistant R7 cells. A comparatively higher amount was observed in R7 cells whereas only traces were detected in their K562 parental cell line.

The presence of MRP1/ABCC1, MRP2/ABCC2, and BCRP/ABCG2 also known to contribute to the MDR phenotype was investigated. Low amounts of MRP1 mRNA were found in both R7 and H295R cells whereas a low level of BCRP was present in R7 cells only. No MRP2 could be detected in both cells lines.

Cytotoxicity Assay Using [³H]Thymidine Incorporation in Surviving Cells

Cells (150, 000/well) were seeded into 24-well plates and incubated at 37° C. in a 5% CO₂ atmosphere.

For H295R cell line, cells were seeded into 24-well plates and, after cell attachment (24 h), culture medium in each well was replaced by medium containing three concentrations (10⁻⁶ M, 10⁻⁶ M, 10⁻⁷ M) of steroid derivatives according to the invention prepared from an initial solution at 10⁻² M in DMSO or the same three concentrations of cyclosporine A prepared from an initial solution at 10⁻² M in water as control. Doxorubicine (DOXO) at 10⁻⁶ M was added shortly thereafter to each well. After incubation for 24 h, the drug-containing medium was replaced by fresh medium containing 1 μCi/mL of [methyl-³H]thymidine (GE Healthcare). After another 24 h incubation at 37° C., the medium containing [³H]thymidine was carefully aspirated from each well and the cells were precipitated by 1 mL of a solution of trichloroacetic acid (TCA) at 10% in water. After incubation for 15 min at 4° C., the medium was carefully aspirated and the precipitate was washed by 500 μL of TCA 5%. Finally the precipitate was solubilized by addition of 300 μL of a solution of sodium deoxycholate (4% in NaOH 0.5 M) (O. Joly-Pharaboz et al., J Steroid Biochem. Mol. Biol. 73 (2000) 237-249). Radioactivity was measured (Tri-Carb 1900 CA, Packard) after addition of liquid scintillation cocktail (Perkin Elmer, Courtaboeuf, France).

For K562/R7 cell lines, cells were distributed into 24-well plates and incubated with 600 μL of medium containing the compounds of the invention or cyclosporine A and doxorubicin at the same concentrations as for H295R cells. After 24 h incubation, 500 μl of the culture medium were removed and replaced by 500 μl of fresh medium containing 1 μCi of [³H]thymidine. Cells were incubated for another 24 hours and pelleted by centrifugation (1000 rpm, 5 min), the medium was aspirated off, cells were washed twice with phosphate-buffered saline (PBS) and radioactivity was measured after TCA precipitation as above.

For both cell lines, each concentration of steroid or cyclosporine A was tested in triplicate and controls, using support medium only, were performed. At least three different experiments were performed for each cell type.

Results were expressed as the percentage of [³H]thymidine incorporation in surviving cells in each well compared to untreated cells (Table 2).

TABLE 2
[³H]thymidine incorporation in surviving cells after treatment with
doxorubicin (10⁻⁶M) in the presence of steroid modulators (10⁻⁶M)

| | |
|---|---|
| Substitutions | [³H]thymidine incorporation (% untreated cells) |

Pregnene skeleton

| | | | |
|---|---|---|---|
| | progesterone | 31.33 ± 3.84 | 37.47 ± 2.47 |
| C7 | 3 | 37.71 ± 6.00 | 45.75 ± 1.96 |
| C17alpha | 9 | 11.59 ± 1.9 | 13.10 ± 0.71 |
| | 10 | 8.14 ± 0.08 | not determined |
| | 11 | 17.93 ± 0.31 | not determined |

Pregnane skeleton

| | | | |
|---|---|---|---|
| | 7 | 25.53 ± 3.27 | not determined |
| C3 and C7 | 18 | 11.46 ± 1.46 | not determined |
| C3 and C11 | 20 | 29.35 ± 0.65 | 25.47 ± 4.7 |
| C7 and C11 | 30 | 14.80 ± 0.80 | 5.72 ± 0.29 |
| | 32 | 13.40 ± 3.10 | 13.26 ± 3.8 |
| | 33 | 3.57 ± 0.88 | 1.36 ± 0.42 |
| | 39 | 13.48 ± 1.30 | 15.92 ± 2.3 |
| | 40 | 18.98 ± 2.22 | 2.20 ± 0.8 |
| | 41 | 9.50 ± 2.72 | 6.09 ± 2.0 |
| C11 and C17alpha | 51 | 12.60 ± 2.81 | 2.74 ± 0.67 |
| | 52 | 14.70 ± 4.32 | 11.42 ± 2.70 |

Cholane skeleton

| | | | |
|---|---|---|---|
| C7 and C12 | 57 | 10.41 ± 0.05 | 6.69 ± 0.01 |
| | 59 + 62 | 3.12 ± 0.1 | 1.51 ± 0.04 |

Control

| | | |
|---|---|---|
| Cyclosporine A | 1.58 ± 0.52 | 2.19 ± 0.30 |

The results listed in Table 2 indicate that the tested compounds of the invention are more active than progesterone to increase the cytotoxicity of doxorubicin. Moreover, some disubstituted derivatives proved to be highly efficient, especially compound 33 and the mixture of compounds 59+62 both showing an activity similar to cyclosporine A.

Cytotoxicity assay using 3-(4,5-dimethylthiazolyl-2)-2,5-diphenyl-tetrazolium bromide (MTT) reagent

K562/R7 cells (10,000/well) were seeded into 96-well plates and incubated with 200 μL of medium containing the compounds of the invention or cyclosporine A at three concentrations (10⁻⁵ M, 10⁻⁶ M 10⁻⁷ M). Doxorubicin at 10⁻⁶ M was then added to each well. After incubation for 72 h, 20 μL of MTT reagent (5 mg/mL in PBS buffer) were added to each well and the plate was further incubated for 4 h at 37° C., allowing viable cells to change the yellow MTT into dark-blue formazan crystals. Supernatants were carefully discarded and 100 μL of isopropanol containing 10% HCL 1M were added to dissolve the formazan crystals. Absorbance in each well was determined at 570 nm using a microplate reader (MultisKan EX, Thermo Electron). Each concentration of steroid or cyclosporine A was tested in triplicate and controls, using support medium only, were performed. At least three different experiments were performed. Results were expressed as the percentage of surviving cells in each well compared to untreated cells.

TABLE 2 bis
Surviving cells measured with MTT after treatment with
doxorubicin (10⁻⁶M) in the presence of steroid modulators (10⁻⁶M)

| | |
|---|---|
| | Surviving cells R7 cells (% untreated |
| Substitutions | cells) |

Pregnane skeleton

| | | |
|---|---|---|
| C6 | 204 | 2.8 ± 0.7 |
| C6 and C11 | 217 | 5.5 ± 0.4 |
| C6 and C11 | 218 | 6.2 ± 1.1 |
| C7 and C11 | 33 | 1.5 ± 0.9 |

Cholane skeleton

| | | |
|---|---|---|
| C6 | 210 | 4.1 ± 1.1 |
| C7 and C12 | 59 | 1.8 ± 0.1 |
| | 60 | 2.2 ± 0.3 |
| | 63 | 0.7 ± 0.06 |
| | 64 | 0.8 ± 0.06 |

Control

| | |
|---|---|
| Cyclosporine A | 1.6 ± 0.5 |

The results listed in table 2bis indicate that compounds 63 and 64 are highly actives and showed an activity higher than that of the reference compound cyclosporine A.

Determination of IC₅₀ for Cytotoxics

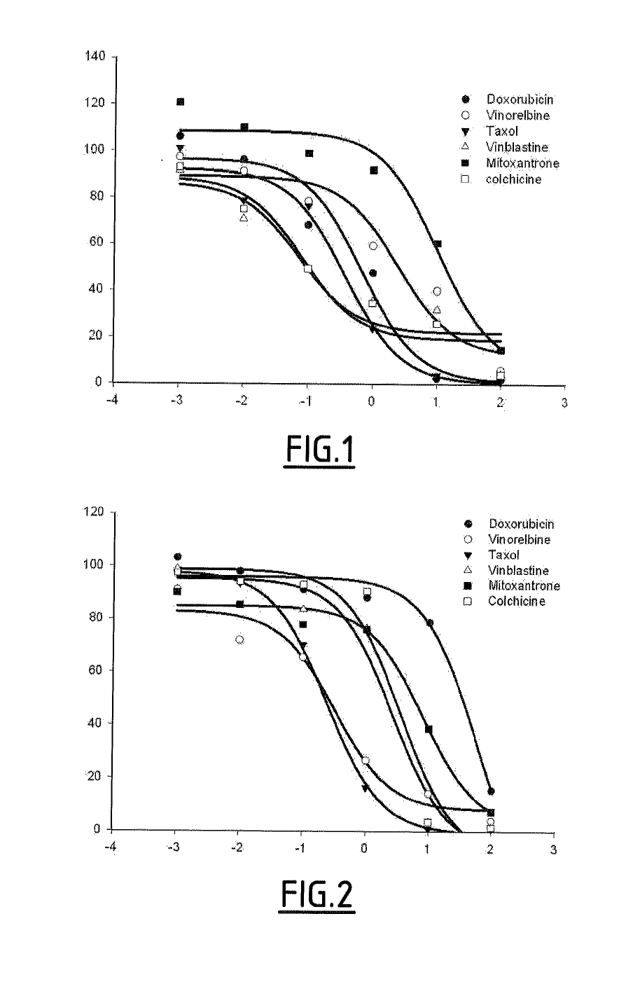

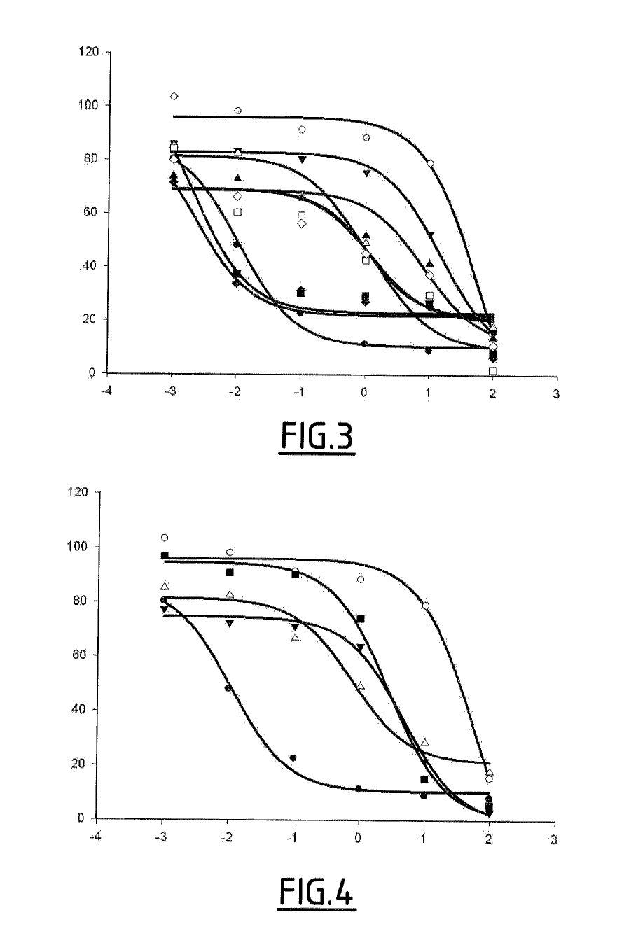

IC₅₀ values, defined as the concentration of cytotoxic drugs inhibiting cell growth by 50% were determined by treatment of resistant K562/R7 and H295R cells (150, 000 cells/well) for 24 h with increasing concentrations of several cytotoxic drugs (from 10⁻⁸ to 10⁻⁴ M) (FIGS. 1 and 2). After treatment, the number of surviving cells was evaluated by the incorporation of [³H]thymidine as described above.

Both cell lines are highly resistant to doxorubicin with IC₅₀=20.6 μM in H295R cells and IC₅₀=63.57 μM in R7 cells. Differences between the two cell lines appeared for the other tested antimitotics. R7 cells are resistant to colchicine, mitoxantrone and vinblastine whereas H295R cells are resistant to mitoxantrone and vinorelbine.

Reversion of Chemoresistance to Doxorubicin by Steroid Modulators

The modulation of IC50 for doxorubicin (DOXO) by the steroid modulators was measured by treatment of resistant R7 and H295R cells (150 000 cells/well) for 24 h with increasing concentrations of DOXO (from 10⁻⁸ to 10⁻⁴ M) in the presence or absence of appropriate concentrations of steroids or of cyclosporine A. (FIGS. 3 and 4). Sensitive parental K562 cells were also treated with the same increasing concentrations of DOXO (from 10⁻⁸ to 10⁻⁴ M) in the absence of steroids, as a control.

The most effective derivatives for decreasing 1050 for doxorubicin were compounds 33 and 51 employed at respectively 0.4 and 0.5 μM (IC50=0.01±0.007 and 0.01±0.008). These values, 10 times lower than that measured for cyclosporine A in the same conditions, indicate a 10-fold potency of compounds 33 and 51 relative to cyclosporine A, in vitro.

Intracellular Accumulation of Daunorubicin Measured by Flow Cytometry

The accumulation of daunorubicin in the presence of steroid modulators according to the invention, i.e. compounds having formula (I), was measured by flow cytometry using a reported procedure (G. Comte et al, J. Med. Chem., 44: 763-768, 2001). K562 or R7 cells (1.10⁻⁶ cells) were incubated for 1 h at 37° C. with 1 mL RPMI 1640 medium containing daunorubicin at 10 μM, in the presence or absence of compounds of the invention (10 μM). The cells were then washed twice with ice-cold PBS and kept on ice until analysis by flow cytometry on a FACS-II (Becton-Dickinson Corp., Mountain View, Calif.). Assays were performed in duplicate, in at least three separate experiments. Cyclosporine A was used as a positive control (I. Raad et al., Bioorg. Med. Chem. 14 (2006) 6979-6987).

TABLE 3
Daunorubicin accumulation in R7 cells in the presence of steroid
modulators expressed in % of the accumulation measured in the
presence of cyclosporine A.

| | Daunorubicin |
|---|---|
| Steroid modulators | accumulation |
| Progesterone | 36.27 ± 4.9 |
| 3 | 108.78 ± 4.27 |
| 7 | 103.0 ± 1.86 |
| 11 | 108.08 ± 6.28 |
| 32 | 103.88 ± 4.53 |
| 33 | 119.25 ± 4.53 |
| 39 | 109.0 ± 1.0 |
| 40 | 89.5 ± 0.5 |
| 41 | 73.3 ± 0.5 |
| 51 | 68.02 ± 15.6 |
| 52 | 102.01 ± 2.0 |
| 53 | 107.0 ± 5.0 |
| 30 | 75.39 ± 0.39 |
| 57 | 74 ± 1.0 |
| 63 | 104.7 ± 7.0 |
| 59 | 85.8 ± 2.6 |
| 60 | 79.8 ± 6.1 |
| 64 | 10.0 ± 1.5 |
| 71 | 50.50 ± 7.5 |
| 74 | 68.0 ± 15.0 |
| 73 | 63.5 ± 5.5 |
| 204 | 87.4 ± 3.5 |
| 210 | 66.7 ± 1.8 |

All the tested compounds of the invention induced an increased daunorubicin accumulation as compared with progesterone. The accumulation of daunorubicin by cells in the presence of compound 33 was more important than with cyclosporine A (119.25±4.53) whereas in the presence of compounds 52, or 53 or of the mixture of 59+62, the accumulation of daunorubicin was equivalent to that observed with cyclosporine A. Moreover, several derivatives in the pregnene/pregnane as well as in the cholane series were as efficient as cyclosporine A in increasing the accumulation of daunorubicin into the cells.

Progesterone Receptor Assays.

The relative binding affinity of steroid modulators to progesterone receptors was measured by a DCC-competitive binding assay as previously described (F. Descotes et al., Breast Cancer Res. Treat., 49 (1998) 135-143). Briefly, aliquots (80 μL) of cytosol (prepared from a pool of breast tumors expressing high level of progesterone receptors) were incubated overnight at 0° C. with 20 μl of [³H]ORG 2058, a synthetic ligand of progesterone receptor (10 000 cpm, 1×10⁻⁹ M, in the absence or in the presence of unlabeled competitors (0.14, 0.7, 1.4 and 2.8×μM) in microtitration plates. At the end of the incubation, free and bound steroids were separated by incubation for 15 min at 4° C. with 100 μL of dextran coated charcoal (DCC) suspension (1.25 g activated charcoal, 125 mg dextran, and 10 mM Tris-HCl, pH 7.4). The plates were centrifuged at 2200 rpm for 15 min. Radioactivities of aliquot (100 μL) of each supernatants were measured (Tri-Carb 1900 CA, Packard) after addition of 2 mL of liquid scintillation cocktail (Perkin Elmer, Courtaboeuf, France). For each concentration of competitor, the radioactivity of bound [³H]ORG2054 (B) is expressed as the percentage of radioactivity bound in the absence of competitor (B°) (FIG. 5).

The curves presented in FIG. 5 show that no significant binding affinity for progesterone receptor could be found for any of the tested derivatives.

Binding to hPXR Receptor

The activation of human pregnane X receptor (hPXR) by the steroid modulators was measured in by Dr P. Balaguer (INSERM U 896, Montpellier, France) using a reported procedure (G. Lemaire et al., Toxicol. Sci. 91 (2006) 501-509). The activation of hPXR by the different tested derivatives was found, in all cases, far lower than that of the reference molecule SR12813 (cholesterol lowering drug) (FIG. 6).

In conclusion, this absence of significant hormonal receptor activation by the tested compounds of the invention provides arguments for the absence of corresponding hormonal side-effects when employed in vivo.

In Vivo Experiments

The in vivo efficiency of the most active (33) derivative was evaluated on SCID (severe combined immunodeficient) mice xenografted with resistant H295R and R7 cells. For each xenograft, four groups of five mice developing a tumour in both flanks were employed. Group 1 received vehicle alone, group 2 received DOXO (1.5 mg/kg/mouse+vehicle), group 3 received (33) derivative (10 mg/kg/mouse solubilised with vehicle) and group 4 received DOXO+derivative. For both H295R and R7 xenografts, mice of group 4 showed a delay in the development of tumour by comparison with mice of groups 1, 2 and 3. Moreover, in mice of group 4, the tumour volume remains low and stable after 40 days of treatment allowing a longer survival time for these mice (FIGS. 7 and 8). These results suggested an in vivo efficiency of (33) derivative as adjuvant to chemotherapy treatment.

1. A method of reversing or inhibiting multidrug resistance in a patient in need thereof, comprising the step of
administering to said patient a therapeutic amount of a compound of formula (I): 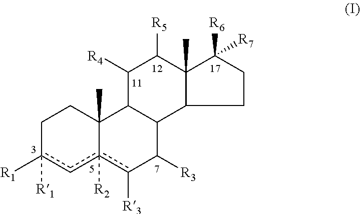
wherein 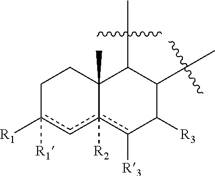
is selected from (Ia), Ib) or (Id): 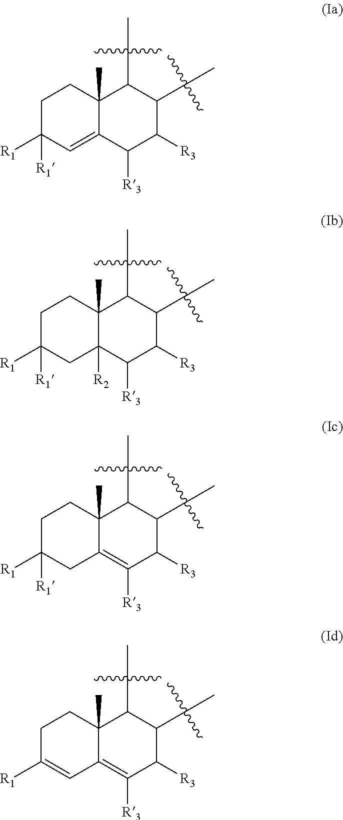
and wherein
R₁ and R₁′ are each independently selected from H, OR₁₉, OC(═O)Ar, OS1R₁₅R₁₆R₁₇, and NR₁₈C(═O)Ar, or together with the carbon atom to which they are attached form ═O, or a 5 to 7 membered heterocycle provided that when 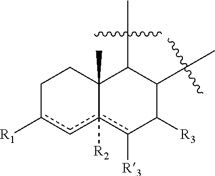
is (Id), R′ cannot be ═O;
R₂ is H;
R₃ and R₃′ are each independently selected from H, OH, (CH₂)ₘOR₈, OR_{B}, ((OCH₂)₂)ₘOR₈, O(CH₂)ₘOR₈, OC(═O)Ar, NHC(═O)Ar, and NHC(═O)(CH₂)ₙAr, wherein said Ar groups are optionally substituted by one to three groups selected from OH, NO₂, N₃, NH₂, and N(CH₃)₂;
R₄ is H, OR₈, O(CH₂)ₘOR₈, ((OCH₂)₂)ₘOR₈, OC(═O)Ar or C(═O)CH₂NH(CH₂)ₜR₈;
R₅ is H, OR₉, OR₈, O(CH₂)ₘOR₈, ((OCH₂)₂)ₘOR₈, or OC(═O)Ar, wherein said Ar group is optionally substituted by one to three NO₂;
R₆ and R₇ are each independently selected from H, CR₁₀R₁₁R₁₂, C(═O)R₁₃, and OR₁₄;
R₈ is a 5 to 7 membered heterocycle, (CH₂)_{S}CN or (CH₂)_{S}NHC(═O)Ar, wherein said Ar is optionally substituted by one to three groups selected from NO₂, N₃ or OH;
R₉ is a 5 to 7 membered heterocycle or (CH₂)ₚOAlk;
R₁₀, R₁₁, and R₁₂ are each independently selected from H, OH, C₁-C₆ alkyl, OC(═O)Ar, OSiR₁₅R₁₆R₁₇, OC(═O)Ar, (CH₂)_{q}OAlk and (CH₂)_{q}C(═O)OAlk, or two of R₁₀, R₁₁, and R₁₂ together form with the carbon atom to which they are attached a 5 to 7 membered heterocycle;
R₁₃ is C₁-C₆ alkyl, (CH₂)ᵣOHet, (CH₂)ᵣSAr or (CH₂)ᵣS Alk;
R₁₄ is H, ((OCH₂)₂)ₘOR₈, O(CH₂)ₘOR₈, a 5 to 7 membered heterocycle or C(═O)Ar;
R₁₅, R₁₆ and R₁₇ are each independently selected from C₁-C₆ alkyl;
R₁₈ is H or C₁-C₆ alkyl;
R₁₉ is H, a 5 to 7 membered heterocycle or (CH₂)ₛNHC(═O)Ar, wherein said Ar is optionally substituted by one to three groups selected from NO₂, N₃ or OH; and
m, n, p, q, r, s and t are each independently selected from 1, 2, 3 or 4;
provided that:
when R₆ is other than H and R₇ is H, then at least one of R₃, R′₃, R₄ and/or R₅ is other than H, and
when R₆ is COCH₃ and R₇ is OH, then at least one of R₃, R′₃, R₄ and/or R₅ is other than H.

2. The method according to claim 1, wherein at least one of R₃, R′₃, R₄ and/or R₅ is other than H.

3. The method according to claim 1, wherein said method is used for reversing or inhibiting multidrug resistance in cancer, or in bacterial, fungal or parasitic infections.

4. The method of claim 1, wherein said compound is administered together with an antitumoral medicine.

5. A method of reversing or inhibiting multidrug resistance in a patient in need thereof, comprising the step of
administering to said patient a therapeutic amount of a compound of formula (II) 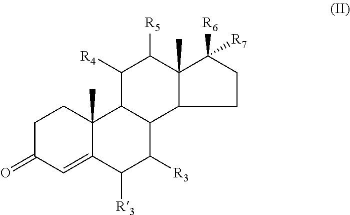
wherein
R₃ and R₃′ are each independently selected from H, OH, (CH₂)ₘOR₈, OR₈, ((OCH₂)₂)₂)ₘOR₈, O(CH₂)ₘOR₈, OC(═O)Ar, NHC(═O)Ar, and NHC(═O)(CH₂)ₙAr, wherein said Ar groups are optionally substituted by one to three groups selected from OH, NO₂, N₃, NH₂, and N(CH₃)₂;
R₄ is H, OR₈, O(CH₂)ₘOR₈, ((OCH₂)₂)ₘOR₈, OC(═O)Ar or C(═O)CH₂NH(CH₂)ₜR₈;
R₅ is H, OR₉, OR₈, O(CH₂)ₘOR₈, ((OCH₂)₂)ₘOR₈, or OC(═O)Ar, wherein said Ar group is optionally substituted by one to three NO₂
R₆ and R₇ are each independently selected from H, CR₁₀R₁₁R₁₂, C(═O)R₁₃, and OR₁₄,
wherein
R₈ is a 5 to 7 membered heterocycle, (CH₂)_{S}CN or (CH₂)_{S}NHC(═O)Ar, wherein said Ar is optionally substituted by one to three groups selected from NO₂, N₃ or OH;
R₉ is a 5 to 7 membered heterocycle or (CH₂)ₚOAlk;
R₁₀, R₁₁, and R₁₂ are each independently selected from H, OH, C₁-C₆ alkyl, OC(═O)Ar, OSiR₁₅R₁₆R₁₇, OC(═O)Ar, (CH₂)_{q}OAlk and (CH₂)_{q}C(═O)OAlk, or two of R₁₀, R₁₁, and R₁₂ together form with the carbon atom to which they are attached a 5 to 7 membered heterocycle, wherein R₁₅, R₁₆ and R₁₇ are each independently selected from C₁-C₆ alkyl;
R₁₃ is C₁-C₆ alkyl, (CH₂)ᵣOHet, (CH₂)ᵣSAr or (CH₂)ᵣSAlk; and
R₁₄ is H, ((OCH₂)₂)ₘOR₈, O(CH₂)ₘOR₈, a 5 to 7 membered heterocycle or C(═O)Ar;
and wherein
m, n, p, q, r, s and t are each independently selected from 1, 2, 3 or 4.

6. A method of reversing or inhibiting multidrug resistance in a patient in need thereof, comprising the step of
administering to said patient a therapeutic amount of a compound of formula (III): 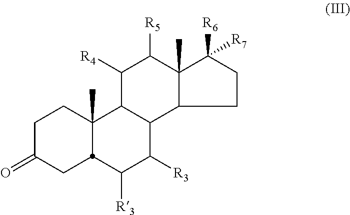
wherein
R₃ and R₃′ are each independently selected from H, OH, (CH₂)ₘOR₈, OR₈, ((OCH₂)₂)ₘOR₈, O(CH₂)ₘOR₈, OC(═O)Ar, NHC(═O)Ar, and NHC(═O)(CH₂)ₙAr, wherein said Ar groups are optionally substituted by one to three groups selected from OH, NO, N₃, NH₂, and N(CH₃)₂;
R₄ is H, OR₈, O(CH₂)ₘOR₈, ((OCH₂)₂)ₘOR₈, OC(═O)Ar or C(═O)CH₂NH(CH₂)ₜR₈;
R₅ is H, OR₉, OR₈, O(CH₂)ₘOR₈, ((OCH₂)₂)ₘOR₈, or OC(═O)Ar, wherein said Ar group is optionally substituted by one to three NO₂;
R₆ and R₇ are each independently selected from H, CR₁₀R₁₁R₁₂, C(═O)R₁₃, and OR₁₄;
wherein
R₈ is a 5 to 7 membered heterocycle, (CH₂)_{S}CN or (CH₂)_{S}NHC(═O)Ar, wherein said Ar is optionally substituted by one to three groups selected from NO₂, N₃ or OH;
R₉ is a 5 to 7 membered heterocycle or (CH₂)ₚOAlk;
R₁₀, R₁₁, and R₁₂ are each independently selected from H, OH, C₁-C₆ alkyl, OC(═O)Ar, OSiR₁₅R₁₆R₁₇, OC(═O)Ar, (CH₂)_{q}OAlk and (CH₂)_{q}C(═O)OAlk, or two of R₁₀R₁₁, and R₁₂ together form with the carbon atom to which they are attached a 5 to 7 membered heterocycle, wherein R₁₅, R₁₆ and R₁₇ are each independently selected from C₁-C₆ alkyl;
R₁₃ is C₁-C₆ alkyl, (CH₂)ᵣOHet, (CH₂)ᵣSAr or (CH₂)ᵣSAlk; and
R₁₄ is H, ((OCH₂)₂)ₘOR₈, O(CH₂)ₘOR₈, a 5 to 7 membered heterocycle or C(═O)Ar;
and wherein
m, n, p, q, r, s and t are each independently selected from 1, 2, 3 or 4.

7. A method of reversing or inhibiting multidrug resistance in a patient in need thereof, comprising the step of
administering to said patient a therapeutic amount of a compound of formula (IV): 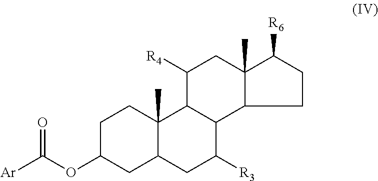
wherein
R₃ is selected from H, OH, (CH₂)ₘOR₈, OR₈, ((OCH₂)₂)ₘOR₈, O(CH₂)ₘOR₈, OC(═O)Ar, NHC(═O)Ar, and NHC(═O)(CH₂)ₙAr, wherein said Ar groups are optionally substituted by one to three groups selected from OH, NO₂, N₃, NH₂, and N(CH₃)₂;
R₄ is H, OR₈, O(CH₂)ₘOR₈, ((OCH₂)₂)ₘOR₈, OC(═O)Ar or C(═O)CH₂NH(CH₂)ₜR₈; and
R₆ is selected from H, CR₁₀R₁₁R₁₂, C(═O)R₁₃, and OR₁₄;
wherein
R₈ is a 5 to 7 membered heterocycle, (CH₂)_{S}CN or (CH₂)_{S}NHC(═O)Ar, wherein said Ar is optionally substituted by one to three groups selected from NO₂, N₃ or OH;
R₁₀, R₁₁, and R₁₂ are each independently selected from H, OH, C₁-C₆ alkyl, OC(═O)Ar, OSiR₁₅R₁₆R₁₇, OC(═O)Ar, (CH₂)_{q}OAlk and (CH₂)_{q}C(═O)OAlk, or two of R₁₀, R₁₁, and R₁₂ together form with the carbon atom to which they are attached a 5 to 7 membered heterocycle, wherein R₁₅, R₁₆ and R₁₇ are each independently selected from C₁-C₆ alkyl;
R₁₃ is C₁-C₆ alkyl, (CH₂)ᵣOHet, (CH₂)ᵣSAr or (CH₂)ᵣSAlk; and
R₁₄ is H, ((OCH₂)₂)ₘOR₈, O(CH₂)ₘOR₈, a 5 to 7 membered heterocycle or C(═O)Ar;
and wherein
m, n, q, r and t are each independently selected from 1, 2, 3 or 4.

8. A method of reversing or inhibiting multidrug resistance in a patient in need thereof, comprising the step of
administering to said patient a therapeutic amount of a compound of formula (V): 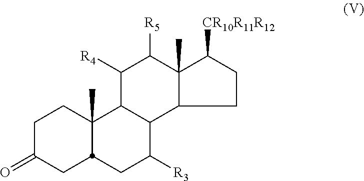
wherein
R₃ is selected from H, OH, (CH₂)ₘOR₈, OR₈, ((OCH₂)₂)ₘOR₈, O(CH₂)ₘOR₈, OC(═O)Ar, NHC(═O)Ar, and NHC(═O)(CH₂)ₙAr, wherein said Ar groups are optionally substituted by one to three groups selected from OH, NO₂, N₃, NH₂, and N(CH₃)₂;
R₄ is H, OR₈, O(CH₂)ₘOR₈, ((OCH₂)₂)ₘOR₈, OC(═O)Ar or C(═O)CH₂NH(CH₂)ₜR₈;
R₅ is H, OR₉, OR₈, O(CH₂)ₘOR₈, ((OCH₂)₂)ₘOR₈, or OC(═O)Ar, wherein said Ar group is optionally substituted by one to three NO₂;
wherein
R₈ is a 5 to 7 membered heterocycle, (CH₂)_{S}CN or (CH₂)_{S}NHC(═O)Ar, wherein said Ar is optionally substituted by one to three groups selected from NO₂, N₃ or OH;
R₉ is a 5 to 7 membered heterocycle or (CH₂)ₚOAlk;
R₁₀, R₁₁, and R₁₂ are each independently selected from H, OH, C₁-C₆ alkyl, OC(═O)Ar, OSiR₁₅R₁₆R₁₇, OC(═O)Ar, (CH₂)_{q}OAlk and (CH₂)_{q}C(═O)OAlk, or two of R₁₀, R₁₁, and R₁₂ together form with the carbon atom to which they are attached a 5 to 7 membered heterocycle, wherein R₁₅, R₁₆ and R₁₇ are each independently selected from C₁-C₆ alkyl;
and wherein
m, n, p, q, s and t are each independently selected from 1, 2, 3 or 4,
and wherein one of R₁₀, R₁₁ and R₁₂ is (CH₂)_{q}OAlk or (CH₂)_{q}C(═O)OAlk.

9. A method of reversing or inhibiting multidrug resistance in a patient in need thereof, comprising the step of
administering to said patient a therapeutic amount of a compound of formula (VI): 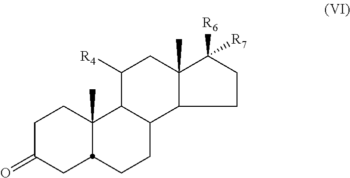
wherein
R₄ is H, OR₈, O(CH₂)ₘOR₈, ((OCH₂)₂)ₘOR₈, OC(═O)Ar or C(═O)CH₂NH(CH₂)ₜR₈;
R₆ and R₇ are each independently selected from H, CR₁₀R₁₁R₁₂, C(═O)R₁₃, and OR₁₄;
wherein
R₈ is a 5 to 7 membered heterocycle, (CH₂)_{S}CN or (CH₂)_{S}NHC(═O)Ar, wherein said Ar is optionally substituted by one to three groups selected from NO₂, N₃ or OH;
R₁₀, R₁₁, and R₁₂ are each independently selected from H, OH, C₁-C₆ alkyl, OC(═O)Ar, OSiR₁₅R₁₆R₁₇, OC(═O)Ar, (CH₂)_{q}OAlk and (CH₂)_{q}C(═O)OAlk, or two of R₁₀, R₁₁, and R₁₂ together form with the carbon atom to which they are attached a 5 to 7 membered heterocycle, wherein R₁₅, R₁₆ and R₁₇ are each independently selected from C₁-C₆ alkyl;
R₁₃ is C₁-C₆ alkyl, (CH₂)ᵣOHet, (CH₂)ᵣSAr or (CH₂)ᵣSAlk; and
R₁₄ is H, ((OCH₂)₂)ₘOR₈, O(CH₂)ₘOR₈, a 5 to 7 membered heterocycle or C(═O)Ar;
and wherein m, q, s, r and t are each independently selected from 1, 2, 3 or 4;
and wherein R₄, R₆ and R₇ are not H.

10. A method of reversing or inhibiting multidrug resistance in a patient in need thereof, comprising the step of
administering to said patient a therapeutic amount of a compound of formula VII) 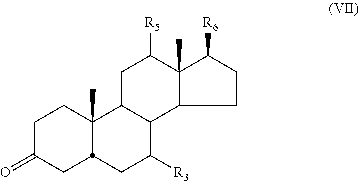
wherein
R₃ is selected from H, OH, (CH₂)ₘOR₈, OR₈, ((OCH₂)₂)ₘOR₈, O(CH₂)ₘOR₈, C(═O)Ar, NHC(═O)Ar, and NHC(═O)(CH₂)ₙAr, wherein said Ar groups are optionally substituted by one to three groups selected from OH, NO₂, N₃, NH₂, and N(CH₃)₂;
R₅ is H, OR₉, OR₈, O(CH₂)ₘOR₈, ((OCH₂)₂)ₘOR₈, or OC(═O)Ar, wherein said Ar group is optionally substituted by one to three NO₂; and
R₆ is selected from H, CR₁₀R₁₁R₁₂, C(═O)R₁₃, and OR₁₄;
and wherein
R₈ is a 5 to 7 membered heterocycle, (CH₂)_{S}CN or (CH₂)_{S}NHC(═O)Ar, wherein said Ar is optionally substituted by one to three groups selected from NO₂, N₃ or OH;
R₉ is a 5 to 7 membered heterocycle or (CH₂)ₚOAlk;
R₁₀, R₁₁, and R₁₂ are each independently selected from H, OH, C₁-C₆ alkyl, OC(═O)Ar, OSiR₁₅R₁₆R₁₇, OC(═O)Ar, (CH₂)_{q}OAlk and (CH₂)_{q}C(═O)OAlk, or two of R₁₀, R₁₁, and R₁₂ together form with the carbon atom to which they are attached a 5 to 7 membered heterocycle, wherein R₁₅, R₁₆ and R₁₇ are each independently selected from C₁-C₆ alkyl, wherein R₁₅, R₁₆ and R₁₇ are each independently selected from C₁-C₆ alkyl;
R₁₃ is C₁-C₆ alkyl, (CH₂)ᵣOHet, (CH₂)ᵣSAr or (CH₂)ᵣSAlk; and
R₁₄ is H, ((OCH₂)₂)ₘOR₈, O(CH₂)ₘOR⁸, a 5 to 7 membered heterocycle or C(═O)Ar;
and wherein m, q, s, p and r are each independently selected from 1, 2, 3 or 4;
and wherein R₅ is not H.

11. A pharmaceutical composition comprising a compound of formula (I): 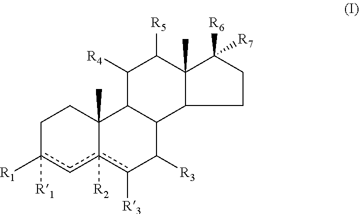
wherein 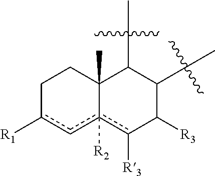
is selected from (Ia), (Ib), (Ic) or (Id): 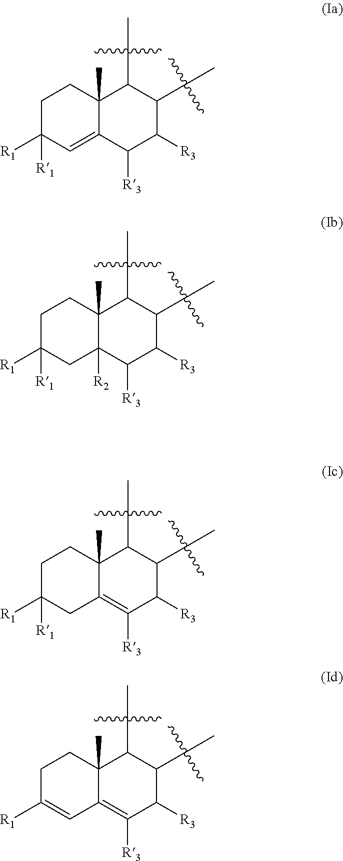
and R1 to R7 are as defined in claim 1,
in admixture with one or more pharmaceutically acceptable excipients, with the exclusion of compounds of formula (I) having the following formulae:

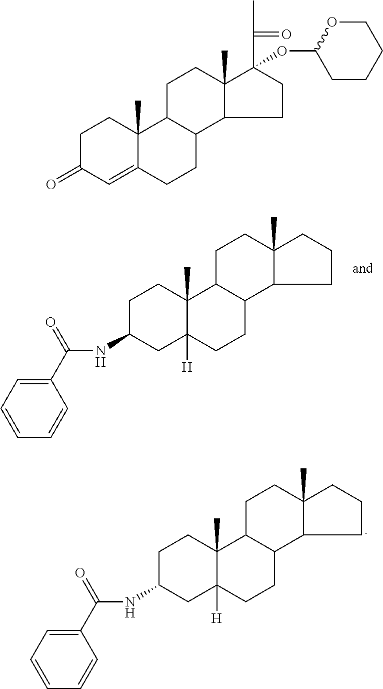

12. A compound having formula (II) 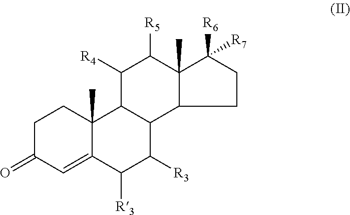
wherein R₃, R′₃, R₄, R₅, R₆ and R₇ are as defined in claim 1, with the exclusion of the R/S mixture of

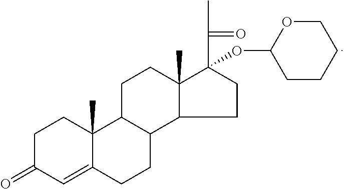

13. The compound of formula (III) 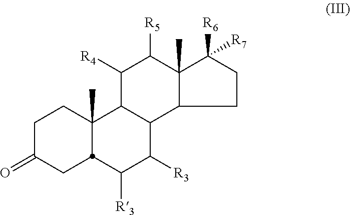
wherein R₃, R′₃, R₄, R₅, R₆ and R₇ are as defined in claim 1, with the exclusion of

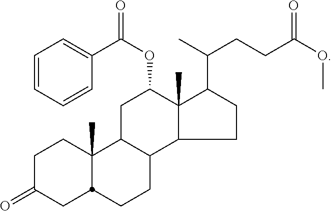

14. The compound of formula (V) 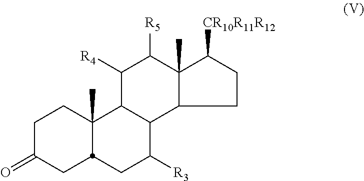
wherein R₃, R₄, R₅, R₁₀, R₁₁ and R₁₂ and are as defined in claim 1, with the exclusion of

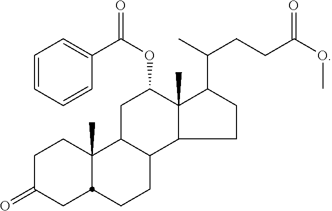

15. The compound of formula (VI): 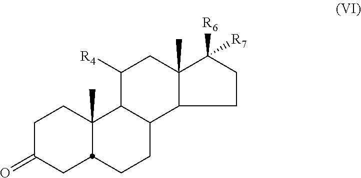
wherein R₄, R₆ and R₇ are as defined in claim 1,
and wherein R₄, R₆, and R₇ are not H.

16. The compound of formula (VII): 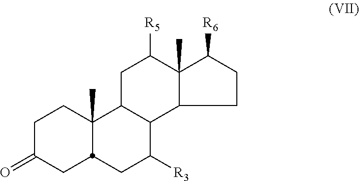
wherein R₃, R₅ and R₆ are as defined in claim 1,
and wherein R₅ is not H. with the exclusion of [chemical structure]
